(19) United States
(12) Patent Application Publication
(10) Pub. No.: US 2025/0179541 A1
(43) Pub. Date: Jun. 5, 2025

(54) METHOD OF PRODUCING CANNABINOIDS

(71) Applicant: GENOMATICA, INC., San Diego, CA (US)

(72) Inventors: Jamison Parker HUDDLESTON, San Diego, CA (US); Andreas SCHIRMER, San Diego, CA (US); Trevor Nelson PURDY, San Diego, CA (US); Benjamin Matthew GRIFFIN, San Diego, CA (US)

(21) Appl. No.: 18/842,666

(22) PCT Filed: Mar. 1, 2023
(86) PCT No.: PCT/US2023/063492

§ 371 Date: Aug. 29, 2024

Related U.S. Application Data
(60) Provisional application No. 63/315,892, filed on Mar. 2, 2022.

(51) Int. Cl. C12P 17/06; C12M 1/00; C12M 1/40; C12N 9/02

(52) U.S. Cl. CPC C12P 17/06; C12M 21/18; C12M 47/06; C12N 9/0004; C12Y 121/03003

(57) ABSTRACT

The present disclosure provides an in vitro method for producing a cannabinoid, the method comprising reacting a N prenylated aromatic compound with a flavin-dependent oxidase in a reaction mixture to form the cannabinoid, wherein the flavin-dependent oxidase is not derived from C. sativa.

### SEQUENCE LISTING

The instant application contains a Sequence Listing which has been submitted electronically in XML format and is hereby incorporated by reference in its entirety. Said Sequence Listing XML, created on Mar. 1, 2023, is named 0171-0009WO1_SL.xml and is 58,558 bytes in size.

### FIELD OF THE INVENTION

The present disclosure provides an in vitro method for producing a cannabinoid, the method comprising reacting a prenylated aromatic compound with a flavin-dependent oxidase in a reaction mixture to form the cannabinoid, wherein the flavin-dependent oxidase is not derived from C. sativa.

### BACKGROUND OF THE INVENTION

Cannabinoids are compounds naturally derived from Cannabis sativa, an annual plant in the Cannabaceae family. The plant contains about 60 cannabinoids in a varied class of chemicals, typically prenylated polyketides derived from fatty acid and isoprenoid precursors, which bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as cannabis, and synthetic cannabinoids, can have activity on cannabinoid receptors and elicit biological responses. Recently, cannabinoids have drawn significant scientific interest in their potential to treat a wide array of disorders, including insomnia, chronic pain, epilepsy, and post-traumatic stress disorder (Babson et al. (2017), Curr Psychiatry Rep 19:23; Romero-Sandoval et al. (2017) Curr Rheumatol Rep 19:67; O'Connell et al. (2017) Epilepsy Behav 70:341-348; Zir-Aviv et al. (2016) Behav Pharmacol 27:561-569). Cannabinoid research and development as therapeutic tools requires production in large quantities and at high purity. However, purifying individual cannabinoid compounds from C. sativa can be time-consuming and costly, and it can be difficult to isolate a pure sample of a compound of interest. Thus, engineered cells can be a useful alternative for the production of a specific cannabinoid or cannabinoid precursor.

Cannabinoids are increasingly being used for pharmaceutical and nutraceutical applications. Cannabinoid compounds used in such applications are almost exclusively obtained from natural sources, for example, from plant tissue. Thus, the prior art discloses obtaining cannabinoid compounds from the trichomes of the C. sativa plant using different solvent extraction methodologies. Some draw backs associated with such methods include poor yields, high costs associated with growing and maintenance of the plant and costs associated with extraction and purification of the plant extract. Security of plants is also an important consideration that adds to the cost of producing pharmaceutical grade cannabinoid compounds.

### SUMMARY OF THE INVENTION

The present disclosure provides an in vitro method for producing a cannabinoid, the method comprising reacting a prenylated aromatic compound with a flavin-dependent oxidase in a reaction mixture to form the cannabinoid, wherein the flavin-dependent oxidase is not derived from C. sativa. In some embodiments, the flavin-dependent oxidase is less than 85% identical to a flavin-dependent oxidase from C. sativa. In some embodiments, the flavin-dependent oxidase does not comprise a disulfide bond. In some embodiments, the flavin-dependent oxidase is immobilized on a solid support. In some embodiments, the flavin-dependent oxidase is prokaryotic or fungal.

The present disclosure provides an in vitro method for producing a cannabinoid, the method comprising reacting a prenylated aromatic compound with a flavin-dependent oxidase in a reaction mixture to form the cannabinoid, and wherein the flavin-dependent oxidase does not comprise a disulfide bond. In some embodiments, the flavin-dependent oxidase is immobilized on a solid support.

In some embodiments, the flavin-dependent oxidase is a prokaryote protein, fungal protein, or derivative thereof.

In some embodiments, flavin-dependent oxidase is a berberine bridge enzyme (BBE)-like enzyme. In some embodiments, the flavin-dependent oxidase comprises: (i) a first amino acid sequence comprising a His residue, wherein an FAD cofactor is covalently attached to the His residue; and (ii) a second amino acid sequence comprising a peptide motif of Formula I:

[Formula I]
X₁-Gly-X₂-Cys-X₃-X₄-X₅-X₆-X₇-X₈-Gly-X₉-X₁₀-X₁₁-Gly-
Gly-Gly-X₁₂-Gly

wherein each X is any amino acid; and wherein the FAD cofactor is covalently attached to the Cys residue, wherein the flavin-dependent oxidase is capable of oxidative cyclization of a prenylated aromatic compound into a cannabinoid. In some embodiments, the flavin-dependent oxidase comprises at least 70% sequence identity to any of SEQ ID NO:1 or SEQ ID NO:3. In some embodiments, the flavin-dependent oxidase comprises at least 70% sequence identity to any of SEQ ID NO:16 or SEQ ID NO:17. In some embodiments, the flavin-dependent oxidase comprises at least 70% sequence identity to any of SEQ ID NOs:34-37. In some embodiments, the flavin-dependent oxidase comprises at least 70% sequence identity to any of SEQ ID NOs:18-33.

In some embodiments, the flavin-dependent oxidase comprises an affinity tag. In some embodiments, the affinity tag is selected from an AUl epitope tag, an AU5 epitope tag, a bacteriophage T7 epitope tag, a bacteriophage V5 epitope tag, a bluetongue virus tag, a calmodulin binding peptide tag, a cellulose binding domain tag, a chitin binding domain tag, a E2 epitope tag, a FLAG epitope tag, a Glu-Glu (EE-tag) tag, a Human influenze hemagglutinin tag, a histidine affinity tag, a HSV epitope tag, a KT3 epitope tag, a Myc epitope tag, a PDZ ligand tag, a polyarginine tag, a polyaspartate tag, a polycysteine tag, an polyhistidine tag, a polyphenylalanine tag, a Protein C tag, an Si tag, an S tag, a streptavadin binding peptide tag, a strep-tag, a TrpE tag, a Universal (HTTPHH) tag, or a VSV-G tag. In some embodiments, the polyhistidine tag is a 6X histidine tag.

In some embodiments, the flavin-dependent oxidase comprises at least 70% sequence identity to any of SEQ ID NO:4, SEQ ID NO:5, SEQ ID NO:6. or SEQ ID NO:15.

In some embodiments, the flavin-dependent oxidase is immobilized on a solid support by an interaction with an affinity tag. In some embodiments, the flavin-dependent oxidase is immobilized on a solid support by an interaction with an antibody. In some embodiments, the flavin-dependent oxidase is immobilized on a solid support by a covalent bond with the solid support.

In some embodiments, the cannabinoid is CBCA, CBC, CBCOA, CBCVA, THCA, THCA, THCV, THCO, THCVA, THCOA, THC, CBDA, CBDV, CBDO, CBDVA, CBDOA, CBD, CBCA, CBCV, CBCO, CBCVA, CBCOA, CBC, cannabinolic acid (CBNA), cannabinol (CBN), cannabicyclol (CBL), cannabivarin (CBV), cannabielsoin (CBE), cannabicitran, an isomer, analog or derivative thereof, or a combination thereof. In some embodiments, the cannabinoid is CBCA, CBC, CBCOA, CBCVA, or THCA.

In some embodiments, the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), cannabigerol (CBG), or combinations thereof. In some embodiments, the prenylated aromatic compound is cannabigerolic acid (CBGA). In some embodiments, the prenylated aromatic compound is cannabigerolic acid (CBGA), and the cannabinoid produced is CBCA, CBDA, or THCA.

In some embodiments, the reaction mixture has a pH of about 4.0 to about 8.0. In some embodiments, the reaction mixture comprises a solvent. In some embodiments, the solvent is one or more of aqueous buffer, dimethyl sulfoxide (DMSO), dimethyl formamide (DMF), iso-propyl alcohol, ethanol and cyclodextrin. In some embodiments, the amount of the solvent in the reaction mixture is between 5% and 30% (w/v). In some embodiments, the reaction mixture comprises a solubility additive. In some embodiments, the solubility additive comprises a nonionic surfactant. In some embodiments, the nonionic surfactant comprises a polysorbate or polyethylene glycol tert-octylphenyl ether.

In some embodiments, the disclosure provides an in vitro method for producing a cannabinoid, the method comprising reacting a prenylated aromatic compound with a flavin-dependent oxidase in a reaction mixture, wherein the flavin-dependent oxidase is immobilized on a solid support, and wherein the flavin-dependent oxidase is a prokaryote protein, fungal protein, or derivative thereof.

In some embodiments, the disclosure provides an in vitro method for producing a cannabinoid, the method comprising reacting a prenylated aromatic compound with a flavin-dependent oxidase in a reaction mixture, wherein the flavin-dependent oxidase is immobilized on a solid support, and wherein the flavin-dependent oxidase comprises at least 70% sequence identity to any of SEQ ID NOs:1-6 or any of SEQ ID NOs:15-37.

In some embodiments, the disclosure provides an in vitro method for producing a cannabinoid, the method comprising: (a) culturing a prokaryote cell or fungal cell comprising a flavin-dependent oxidase in a fermenter,; (b) lysing the prokaryote cell or fungal cell to form a lysate comprising the flavin-dependent oxidase; (c) adding a prenylated aromatic compound to the flavin-dependent lysate in vitro, whereby the prenylated aromatic compound can interact with the flavin-dependent oxidase to fonn the cannabinoid; and (d) recovering the cannabinoid. In some embodiments, the prenylated aromatic compound are added to the flavin-dependent oxidase after one or more impurities in the lysate have been removed. In some embodiments, the flavin-dependent oxidase does not comprise a disulfide bond.

In some embodiments, the disclosure provides an in vitro method for producing a cannabinoid, the method comprising: (a) culturing a prokaryote cell or fungal cell comprising a flavin-dependent oxidase in a fermenter, wherein the flavin-dependent oxidase does not comprise a disulfide bond; (b) lysing the prokaryote cell or fungal cell to fonn a lysate comprising the flavin-dependent oxidase; (c) adding the lysate to a chromatography apparatus comprising a solid support, whereby the flavin-dependent oxidase is immobilized onto the solid support; (d) adding a prenylated aromatic compound to the chromatography apparatus, whereby the prenylated aromatic compound can interact with the flavin-dependent oxidase to form the cannabinoid; and (e) recovering the cannabinoid.

In some embodiments, the disclosure provides a system for producing cannabinoid products, comprising: (a) a fermenter holding a cell culture medium comprising prokaryote cells or fungal cells producing a flavin-dependent oxidase wherein the flavin-dependent oxidase does not comprise a disulfide bond; (b) a means for lysing the prokaryote cells or fungal cells to form a lysate comprising the flavin-dependent oxidase; and (c) an apparatus containing a reaction mixture configured to interact the immobilized flavin-dependent oxidase with a prenylated aromatic compound to form a cannabinoid which is produced by the flavin-dependent oxidase.

In some embodiments, the disclosure provides a system for producing cannabinoid products, comprising: (a) a fermenter holding a cell culture medium comprising prokaryote cells or fungal cells producing a flavin-dependent oxidase wherein the flavin-dependent oxidase does not comprise a disulfide bond; (b) a means for lysing the prokaryote cells or fungal cells to form a lysate comprising the flavin-dependent oxidase; (c) an apparatus containing a reaction mixture configured to immobilize the flavin-dependent oxidase onto a solid support, and then interact the immobilized flavin-dependent oxidase with a prenylated aromatic compound to form a cannabinoid which is produced by the flavin-dependent oxidase. In some embodiments, the disclosure provides a biphasic composition comprising: (a) a prenylated aromatic compound in a first phase, wherein the first phase comprises an organic solvent; (b) a flavin-dependent oxidase in a second phase, wherein the second phase comprises an aqueous solvent, and wherein the flavin-dependent oxidase does not comprise a disulfide bond.

In some embodiments, the disclosure provides a biphasic composition comprising: (a) a prenylated aromatic compound in a first phase, wherein the first phase comprises an organic solvent; (b) a flavin-dependent oxidase immobilized on a solid support in a second phase, wherein the second phase comprises an aqueous solvent, and wherein the flavin-dependent oxidase does not comprise a disulfide bond.

### BRIEF DESCRIPTION OF THE FIGURES

FIG. 1 is the UV-Spectra of Select Fractions of synthesized cannabinoid as found in Example 1.

FIG. 2 demonstrates the ratios of CBLC/MS quantification of CBCA and CBGA in various fractions.

### DETAILED DESCRIPTION OF THE INVENTION

As used herein, unless otherwise stated, the singular forms “a,” “an,” and “the” include plural reference. Thus, for example, a reference to “a cell” includes a plurality of cells, and a reference to “a molecule” is a reference to one or more molecules.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The present disclosure provides for enzyme catalyzed synthesis of cannabinoids in a cell-free environment using a flavin-dependent oxidase that is not derived from C. sativa, e.g., a flavin-dependent oxidase obtained from a prokaryote, wherein the flavin-dependent oxidase is immobilized on a solid support. In some embodiments, the present disclosure provides for enzyme catalyzed synthesis of cannabinoids in a cell-free environment using a flavin-dependent oxidase that is not derived from C. sativa, e.g., a flavin-dependent oxidase obtained from a fungus, wherein the flavin-dependent oxidase is immobilized on a solid support. Also described is an apparatus for the ex vivo manufacture of cannabinoids.

In some embodiments, the present disclosure provides an in vitro method for producing a cannabinoid, the method comprising reacting a prenylated aromatic compound with a flavin-dependent oxidase in a reaction mixture to form the cannabinoid, wherein the flavin-dependent oxidase is not derived from C. sativa. The phrase “not derived from C. sativa” will include those enzymes which were not isolated, cloned, expressed from, or derived from C. sativa. In some embodiments, the term “derived from C. sativa” includes any protein which is less than 85%, less than 80%, less than 70%, less than 65%, less than 60%, less than 55% or less than 55% identical to a flavin-dependent oxidase from C. sativa. In some embodiments, the flavin-dependent oxidase is less than 85% identical to a flavin-dependent oxidase from C. sativa. Thus, the present invention found that flavin-dependent oxidase significantly distinct from those found in C. sativa could be used to produce a cannabinoid in vitro. These flavin-dependent oxidases can be isolated from, e.g., plants (other than C. sativa, e.g., algae), other eukaryotes such as animals (e.g., mammals, insects, reptiles), fungi (e.g., yeast, molds), protists (e.g., amoebae, spyrogea). In some embodiments, the flavin-dependent oxidase is prokaryotic or fungal. In some embodiments, the flavin-dependent oxidase is expressed in a prokaryote cell or a fungal cell.

In some embodiments, the flavin-dependent oxidase that is not a flavin-dependent oxidase from C. sativa does not comprise a disulfide bond. In some embodiments, the flavin-dependent oxidase is immobilized on a solid support.

In some embodiments, the disclosure provides an in vitro method for producing a cannabinoid, the method comprising reacting a prenylated aromatic compound with a flavin-dependent oxidase in a reaction mixture to form the cannabinoid, wherein the flavin-dependent oxidase is immobilized on a solid support, and wherein the flavin-dependent oxidase does not comprise a disulfide bond.

In some embodiments, the present disclosure provides that a flavin-dependent oxidase from a prokaryote or fungi that is suitable for catalyzing the conversion of a prenylated aromatic compound into a cannabinoid. Thus, in some embodiments, the disclosure provides an in vitro method for producing a cannabinoid, the method comprising reacting a prenylated aromatic compound with a flavin-dependent oxidase, wherein the flavin-dependent oxidase is a prokaryote protein or fungal protein or derivative thereof. The disclosure provides that this catalyzing reaction can occur when the flavin-dependent oxidase is immobilized onto a solid support. The disclosure provides that this catalyzing reaction can occur, even though the flavin-dependent oxidase does not comprise a disulfide bond.

In some embodiments, the disclosure provides an in vitro method for producing a cannabinoid, the method comprising reacting a prenylated aromatic compound with a flavin-dependent oxidase in a reaction mixture, wherein the flavin-dependent oxidase is immobilized on a solid support, and wherein the flavin-dependent oxidase is a prokaryote protein, fungal protein or derivative thereof.

In some embodiments, the disclosure provides an in vitro method for producing a cannabinoid, the method comprising reacting a prenylated aromatic compound with a flavin-dependent oxidase in a reaction mixture, wherein the flavin-dependent oxidase is immobilized on a solid support, and wherein the flavin-dependent oxidase comprises at least 70% sequence identity to any of SEQ ID NOs:1-6 and 15-37. In some embodiments, the flavin-dependent oxidase comprises at least 70% sequence identity to SEQ ID NO:1 or SEQ ID NO:3. In some embodiments, the flavin-dependent oxidase comprises at least 70% sequence identity to any of SEQ ID NO:16 or SEQ ID NO:17. In some embodiments, the flavin-dependent oxidase comprises at least 70% sequence identity to any of SEQ ID NOs:34-37. In some embodiments, the flavin-dependent oxidase comprises at least 70% sequence identity to any of SEQ ID NOs:18-33.

In the context of a protein or polypeptide, a disulfide bond (sometimes called a “S-S bond” or “disulfide bridge”) refers to a covalent bond between two cysteine residues, typically formed through oxidation of the thiol groups on the cysteines. Proteins comprising disulfide bonds, e.g., endogenous to plants, can be unstable in prokaryote host cells as the disulfide bonds are often disrupted due to the reducing environment in prokaryote cells. In some embodiments, cannabinoid synthases from C. sativa are substantially unstable in a prokaryote cell, e.g., a bacterial cell, e.g., an F. coli cell. As used herein, “unstable” protein can refer to proteins that are non-functional, denatured, and/or degraded rapidly, resulting in catalytic activity that is greatly reduced relative to the activity found in its native host cell, e.g., C. sativa plants. In some embodiments, the lack of a disulfide bond in the flavin-dependent oxidase advantageously allows for its soluble and active expression by a prokaryote, e.g., bacterial, host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times. or at least 100 times more of the flavin-dependent oxidase that does not comprise a disulfide bond as compared with a flavin-dependent oxidase that comprises a disulfide bond, e.g., a wild-type cannabinoid synthase from C. saliva.

In some embodiments, the flavin-dependent oxidase as described herein is not glycosylated. As used herein, glycosylation refers to the addition of one or more sugar molecules to another biomolecule, e.g., a protein or polypeptide. Glycosylation can play an important role in the folding, secretion, and stability of proteins (see, e.g., Drickamer and Taylor, Introduction to Glycobiology (₂“a ed.), Oxford University Press, USA). In spite of the role of glycosylation present in eukaryote cells, in some embodiments the present disclosure has found that non-glycosylated flavin-dependent oxidase remain functional, even when immobilized on a solid support. The disclosure provides that this catalyzing reaction can occur when the flavin-dependent oxidase lacking glycosylation is immobilized onto a solid support. The disclosure provides that this catalyzing reaction can occur, even though the flavin-dependent oxidase is not glycosylated.

Glycosylation mechanisms and patterns in bacteria and eukaryotes are distinct from one another. Moreover, the most common type of glycosylation, N-linked glycosylation, occurs in eukaryotes but not in bacteria. Thus, prokaryote cells, e.g., bacterial cells, are generally not suitable for the production of eukaryotic proteins that are glycosylated, e.g., the cannabinoid synthases from C. sativa. In some embodiments, the lack of glycosylation in the flavin-dependent oxidase further advantageously allows for its soluble and active expression by a bacterial host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more (e.g., by weight) of the flavin-dependent oxidase that is not glycosylated, compared with a flavin-dependent oxidase that is glycosylated, e.g., a wild-type cannabinoid synthase from C. sativa.

In some embodiments, the flavin-dependent oxidase is a berberine bridge enzyme (BBE)-like enzyme. BBE-like enzymes are described, e.g., in Daniel et al. (2017), Arch Biochem Biophys 632:88-103 and include protein family domains (Pfams) PF08031 (berberine-bridge domain) and PF01564 (flavin adenine dinucleotide (FAD)-binding domain). In general, a BBE-like enzyme comprises a FAD binding module that is formed by the N- and C-terminal portions of the protein, and a central substrate binding domain that, together with the FAD cofactor, provides the environment for efficient substrate binding, oxidation and cyclization. A non-limiting list of BBE-like enzymes and are described in Table 1. It will be understood by one of ordinary skill in the art that, in some embodiments, a BBE-like enzyme binds a flavin mononucleotide (FMN) in addition to or instead of FAD.

TABLE 1
Exemplary BBE-like enzymes with published structures.

| Protein | Organism | PDB Entries | Cofactor(s), ligand(s) |
|---|---|---|---|
| Reticuline Oxidase/ | Eschscholzia | 3D2D, 3D2H, 3D2J, | FAD, (S)-reticuline, (S)-scoulerine, |
| Dehydrogenase | californica | 3FW7, 3WF8, 3WF9, | dehydroscoulerine |
| | | 3WFA, 3GSY, 4EC3, | |
| | | 4PZF | |
| Pollen Allergen PhI | Phleum pratense | 3TSH, 3TSJ, 4PVE, | FAD |
| p 4.0202 | | 4PVH, 4PVK, 4PVJ, | |
| | | 4PWB, 4PWC, | |
| MaDA | Morus alba | 6JQH | FAD |
| THCA Synthase | Cannabis sativa | 3VTE | FAD |
| Pyrimidine | Escherichia coli K- | 6SGG, 6SGL, 6SGM, | FMN, 2,4-dimethoxypyrimidine |
| monooxy genase | 12 | 6SGN, 6TEE, 6TEF, 6TEG | |
| RutA | | | |
| Caerulomycin | Actinoalloteichus sp. | 5I1V, 5I1W | FAD, 6-(hydroxymethyl)[2,2′- |
| Oxidase K (CrmK) | WH1-2216-6 | | bipyridin]-4-ol, 4-hydroxy [2,2′- |
| | | | bipyridine]-6-carbaldehyde |
| Tirandamycin | Streptomyces sp. | 2Y08, 2Y3R, 2Y3S, 2Y4G | FAD, Tirandamycin D, |
| Oxidase L (TamL) | 307-9 | | Tirandamycin E |
| EncM | Streptomyces | 3W8W, 3W8X, 3W8Z, | FAD, 6,6,6-trifluoro-1- |
| | maritimus | 4XLO, 6FOQ, 6FOW, | phenylhexane-1,3,5-trione, (7S)-7- |
| | | 6FP3, 6FY8, 6FY9, 6FYA, | hydroxy-1-phenyloctanc-1,3,5- |
| | | 6FYB, 6FYC, 6FYD, | trione |
| | | 6FYE, 6FYF, 6FYG | |

In some embodiments, the flavin-dependent oxidase has substantial structural similarity with a cannabinoid synthase from C. sativa, e.g., Δ9-tetrahydrocannabinolic acid synthase (THCAS), but is distinct from THCAS, e.g., the flavin-dependent oxidase does not comprise a disulfide bond. THCAS utilizes a FAD cofactor when catalyzing the conversion of substrate CBGA to THCA. In some embodiments, the enzyme comprises a structurally similar active site as a cannabinoid synthase from C. sativa, e.g., THCAS. As used herein, the term “active site” refers to one or more regions in an enzyme that may be important for catalysis, substrate binding, and/or cofactor binding. In some embodiments, the flavin-dependent oxidase has less than 80%, less than 70%, less than 60%, less than 50% or less than 50% identity to THCAS from C. sativa.

In some embodiments, the flavin-dependent oxidase comprises: (i) a first amino acid sequence comprising a His residue, wherein an FAD cofactor is covalently attached to the His residue; and (ii) a second amino acid sequence comprising a peptide motif of Formula I:

[Formula I]
X₁-Gly-X₂-Cys-X₃-X₄-X₅-X₆-X₇-X₈-Gly-X₉-X₁₀-X₁₁-Gly-
Gly-Gly-X₁₂-Gly

wherein each X is any amino acid; and wherein the FAD cofactor is covalently attached to the Cys residue, and wherein the flavin-dependent oxidase is capable of oxidative cyclization of the prenylated aromatic compound into the cannabinoid.

In some embodiments, the flavin-dependent oxidase comprises: Ala at position X₁; Thr, Ser, Arg, Val, Gly, Phe, or Asn at position X₂; Pro, Ala, Gly, Tyr, or Phe at position X₃; Thr, Ser, Ala, Asp, Gly, Asn, or Arg at position X₄; Val or Ile at position X₅; Gly, Ala, Cy s, Arg, or Asn at position X₆; Ile, Val, Ala, Leu, Met, or Pro at position X₇; Ala, Gly, Ser, Thr, or Tyr at position X₈; Leu, His, Phe, Tyr, Ile, Val, or Trp at position X₉; Thr, Val, Leu, Ile, or Ala at position X₁₀; Leu, Gln, Ser, Thr, Cys, or Met at position X₁₁; Ile, Tyr, Leu, Trp, Val, Phe, Met, His, or Gln at position X₁₂; or any combination thereof.

In some embodiments, the peptide motif comprises:

X₁-Gly-X₂-Cys-Pro-Thr-Val-Gly-X₇-X₈-Gly-Leu-Thr-
Leu-Gly-Gly-Gly-X₁₂-Gly.

In some embodiments, X₂ is Thr or Ser; X₇ is Ile or Val; X₈ is Ala, Gly, or Ser; and X₁2 is Ile, Tyr, or Leu.

In some embodiments, the flavin-dependent oxidase as used herein has at least 30% sequence identity to SEQ ID NO: 1, 3, 16, or 17. In some embodiments, the flavin-dependent oxidase has at least 40% sequence identity to SEQ ID NO: 1, 3, 16, or 17. In some embodiments, the flavin-dependent oxidase has at least 50% sequence identity to SEQ ID NO: 1, 3, 16, or 17. In some embodiments, the flavin-dependent oxidase has at least 60% sequence identity to SEQ ID NO: 1, 3, 16, or 17. In some embodiments, the flavin-dependent oxidase has at least 70% sequence identity to SEQ ID NO: 1, 3, 16, or 17. In some embodiments, the flavin-dependent oxidase has at least 80% sequence identity to SEQ ID NO: 1, 3, 16, or 17. In some embodiments, the flavin-dependent oxidase has at least 85% sequence identity to SEQ ID NO: 1, 3, 16, or 17. In some embodiments, the flavin-dependent oxidase has at least 90% sequence identity to SEQ ID NO: 1, 3, 16, or 17. In some embodiments, the flavin-dependent oxidase has at least 95% sequence identity to SEQ ID NO: 1, 3, 16, or 17.

In some embodiments, the flavin-dependent oxidase as used herein has at least 30%, at least 40%, at least 50%, at least 60%, at least 70%, at least 80%, at least 90% or at least 95% sequence identity to any one of SEQ ID NOS:1-6 or 15-37.

In some embodiments, the disclosure provides a flavin-dependent oxidase comprises at least one amino acid variation as compared to a wild type flavin-dependent oxidase, wherein the flavin-dependent oxidase does not comprise a disulfide bond, and wherein the flavin-dependent oxidase is capable of oxidative cyclization of a prenylated aromatic compound into a cannabinoid. In some embodiments, the flavin-dependent oxidase comprises at least 70%, at least 80%, at least 85%, or at least 90% sequence identity to SEQ ID NO:1, and wherein the at least one amino acid variation comprises a substitution at position V136, S137, T139, L144, Y249, F313, Q353, or a combination thereof, wherein the amino acid position corresponds to SEQ ID NO:1.

In some embodiments, the flavin-dependent oxidase comprises a variation at amino acid position V136, S137, T139, L144, Y249, F313, Q353, or a combination thereof, wherein the amino acid position corresponds to SEQ ID NO:1. In some embodiments, the variation comprises an amino acid substitution selected from V136C, S137P, T139V, L144H, Y249H, F313A, Q353N, or a combination thereof. In some embodiments, the variation comprises a T139V substitution.

In some embodiments, the flavin-dependent oxidase comprises at least 70%, at least 80%, at least 85%, or at least 90% sequence identity to SEQ ID NO:3, and wherein the at least one amino acid variation comprises: a substitution at position W58, M101, L104, 1160, G161, Δ163, V167, L168, Δ171, N267, L269, 1271, Y273, Q275, Δ281, L283, C285, E287, V323, V336, Δ338, G340, L342, E370, V372, Δ398, N400, H402, D404, V436, T438. or a combination thereof, wherein the amino acid position corresponds to SEQ ID NO:3. In some embodiments, the flavin-dependent oxidase comprises at least 70% sequence identity to SEQ ID NO:3, e.g., at least 80% sequence identity, at least 85% sequence identity, or at least 90% sequence identity to SEQ ID NO: 3, and wherein the at least one amino acid variation comprises a deletion of about 5 to about 50 amino acid residues at an N-terminus of SEQ ID NO:3, optionally comprising an amino acid substitution at position W58, M101, L104, 1160, G161, Δ163, V167, L168, Δ171, N267, L269, 1271, Y273, Q275, Δ281, L283, C285, E287, V323, V336, Δ338, G340, L342, E370, V372, Δ398, N400, H402, D404, V436, T438, or a combination thereof, wherein the amino acid position corresponds to SEQ ID NO:3. In some embodiments, the flavin-dependent oxidase comprises at least 70%, at least 80%, at least 85%, or at least 90% sequence identity to SEQ ID NO:16 or 17, optionally comprising an amino acid substitution at position W58, M101, L104, 1160, G161, Δ163, V167, L168, Δ171, N267, L269, 1271, Y273, Q275, Δ281, L283, C285, E287, V323, V336, Δ338, G340, L342, E370, V372, Δ398, N400, H402, D404, V436, T438, or a combination thereof, wherein the amino acid position corresponds to SEQ ID NO:3.

In some embodiments, the flavin-dependent oxidase comprises an amino acid substitution selected from W58Q, W58H, W58K, W58G, W58V, M101A, M101S, M101F, M101Y, L104M, L104H, I160V, G161C, G161A, G161Q, G161L, Δ163G, V167F, L168S, L168G, Δ171Y, Δ171F, N267V, N267M, N267L, L269M, L269T, L269A, L269R, I271H, I271R. Y273I, Y273R, Q275K, Q275R, Δ281R, L283V, C285L, E287H, E287L, V323F, V323Y, V336F, Δ338I, G340L, L342Y, E370M, E370Q, V372A, V372E, V372I, V372L, V372T, V372C, Δ398E, Δ398V, N400 W, H402T, H402I, H402V, H402A, H402M, H402Q, D404S, D404T, D404A, V436L, T438A, T438Y, T438F, or a combination thereof, wherein the amino acid position corresponds to SEQ ID NO:3. In some embodiments, the variation comprises an amino acid substitution selected from T438A, T438Y, N400 W, D404A, or a combination thereof, wherein the amino acid position corresponds to SEQ ID NO:3.

In some embodiments, the variation in the flavin-dependent oxidase comprises an amino acid substitution at position D404 and an amino acid substitution at position L269, Y273, Q275, L283, C285, V323, E370, V372, N400, H402, T438, or a combination thereof, wherein the amino acid position corresponds to SEQ ID NO:3. In some embodiments, the variation comprises D404A and one of: L269R, L269T, Q275R, Y273R, L283V, C285L, V323F, V323Y, E370M, E370Q, V372I, N400 W, H402A, H4021, H402M, H402T, H402V, T438A, T438F, or T438Y.

In some embodiments, the variation in the flavin-dependent oxidase comprises an amino acid substitution at position D404, an amino acid substitution at position T438, and an amino acid position L269, Y273, Q275, L283, C285, V323, E370, V372, N400, H402, T438, or a combination thereof, wherein the amino acid position corresponds to SEQ ID NO:3.

In some embodiments, the variation comprises: a) D404A, T438F, and N400 W; b) D404A, T438F, and V323F; c) D404A, T438F, and V323Y; d) D404A, T438F, and E370M; e) D404A, T438F, and H402I; f) D404A, T438F, and E370Q; g) D404A, T438F, and C285L; h) T438F, N400 W, and D404S; i) T438F, V323Y, and D404S; j) T438F, H402I, and D404S; k) T438F, E370Q, and D404S; 1) D404A, T438F, V372I, and N400 W; m) D404A, T438F, V323Y, and N400 W; n) D404A, T438F, E370Q, and N400 W; o) D404A, T438F, V323Y, and E370M; p) D404A, T438F, E370M, and N400 W; q) D404A, T438F, V323F, and H402I; r) D404A, T438F, C285L, and N400 W; s) D404A, T438F, V323F, and N400 W; t) D404A, T438F, E370Q, and H402T; u) D404A, T438F, N400 W, and H402T; v) D404A, T438F, V323F, and H402T; w) D404A, T438F, C285L, and V323F; x) D404A, T438F, L283V, and N400 W; y) D404A, T438F, V323F, and E370M; z) D404A, T438F, Q275R, and N400 W; aa) D404A, T438F, V323Y, and H402T; bb) D404A, T438F, V323F, and V372I; cc) D404A, T438F, C285L, and V323Y; dd) D404A, T438F, E370Q, and H402I; ee) D404A, T438F, V323Y, and E370Q; ff) D404A, T438F, Y273R, and V323Y; gg) D404A, T438F, Y273R, and N400 W; hh) D404A, T438F, Y273R, and V323F; ii) D404A, T438F, E370M, and H402T; jj) D404A, T438F, L269T, and N400 W; kk) D404A, T438F, Q275R, and V323Y; 11) D404A, T438F, V323Y, and H402I; mm) D404A, T438F, V323F, and E370Q; nn) D404A, T438F, Y273R, and Q275R; oo) D404A, T438F, C285L, and E370Q; pp) D404A, T438F, L283V, and V323Y; qq) D404A, T438F, Y273R, and H4021; rr) D404A, T438F, L269T, and E370M; ss) D404A, T438F, C285L, and H402T; tt) D404A, T438F, L269R, and N400 W; uu) D404A, T438F, Y273R, and C285L; vv) D404A, T438F, L283V, and H402I; ww) D404A, T438F, Q275R, and E370Q; xx) D404A, T438F, V372I, and H402I; yy) D404A, T438F, L283V, and E370Q; or zz) D404A, T438F, V372I, and H402T; wherein the amino acid position corresponds to SEQ ID NO:3.

In some embodiments, the flavin-dependent oxidase does not comprise a variation at any of amino acid positions Y374, Y435, and N437, wherein the amino acid position corresponds to SEQ ID NO:3.

In some embodiments, the variation in the flavin-dependent oxidase comprises a deletion of about 5 to about 50 amino acid residues at the N-terminus of SEQ ID NO:3. In some embodiments, the variation in the flavin-dependent oxidase comprises a deletion of about 10 to about 40 amino acid residues at the N-terminus of SEQ ID NO:3. In some embodiments, the variation in the flavin-dependent oxidase comprises a deletion of about 12 to about 35 amino acid residues at the N-terminus of SEQ ID NO:3. In some embodiments, the variation in the flavin-dependent oxidase comprises a deletion of about 14 to about 30 amino acid residues at the N-terminus of SEQ ID NO:3.

In some embodiments, the flavin-dependent oxidase comprises: (i) at least 70% sequence identity to SEQ ID NO:3; and (ii) substitutions at amino acid positions Q275, C285, V323, E370, V372, N400, D404, and T438, wherein the amino acid positions correspond to SEQ ID NO:3. In some embodiments, the flavin-dependent oxidase comprises at least 80% sequence identity to SEQ ID NO:3 and (ii) substitutions at amino acid positions Q275, C285, V323, E370, V372. N400, D404, and T438, wherein the amino acid positions correspond to SEQ ID NO:3. In some embodiments, the flavin-dependent oxidase comprises at least 90% sequence identity to SEQ ID NO:3 and (ii) substitutions at amino acid positions Q275, C285, V323, E370, V372, N400, D404, and T438, wherein the amino acid positions correspond to SEQ ID NO:3. the substitutions comprise Q275R, C285L, V323Y, E370Q, V372I, N400 W, D404A, and T438F, wherein the amino acid positions correspond to SEQ ID NO:3.

In some embodiments, the flavin-dependent oxidase comprises a substitution at amino acid position substitutions at amino acid positions Q275, C285, V323, E370, V372, N400, D404, and T438, and additional substitutions at one or more of L269, 1271, R275, Δ281, L285, or combination thereof. In some embodiments, the substitution at L269 is L269M; the substitution at 1271 is 1271H; the substitution at R275 is R275Q; the substitution at Δ281 is Δ281R; and the substitution at L285 is L285C.

In some embodiments, the flavin-dependent oxidase comprises at least 70%, 80%, 90% or 95% sequence identity to SEQ ID NO:3 with a substitution at amino acid positions Q275, C285, V323, E370, V372, N400, D404, and T438, and additional substitutions at one or more of comprising substitution(s): (1) 1271H;
(2) L269M;
(3) Δ281R;
(4) L285C;
(5) R275Q;
(6) L269M and 1271H;
(7) 1271H and R275Q;
(8) R275Q and L285C;
(9) L269M and R275Q;
(10) L269M and Δ281R; or
(11) 1271H and L285C,
wherein the amino acid positions correspond to SEQ ID NO:3.

In some embodiments, the flavin-dependent oxidase comprises at least 70%, 80%, 90% or 95% sequence identity to SEQ ID NO:3 with a substitution at amino acid positions Q275, C285, V323, E370, V372, N400, D404, and T438, and additional substitutions comprising one or more of L269M, I271H, , wherein the amino acid positions correspond to SEQ ID NO:3. In some embodiments, the substitutions comprise Q275R, C285L, V323Y, E370Q, V372I, N400 W, D404A, T438F, L269M, and 1271H.

In some embodiments, the flavin-dependent oxidase comprises at least 70%, 80%, 90% or 95% sequence identity to SEQ ID NO:3 with a substitution at amino acid positions Q275, C285, V323, E370, V372, N400, D404, and T438, and additional substitutions comprising one or more of E159H, E159N, E159A, E159R, E159Y, E159K, E159G, T268S, Δ272V, Δ2721, Δ272C, Δ272L, Δ272M, R275A, R275N, R275Q, M279L, M279C, F322 W, T325N, T325Q, M326Y, M326S, M326F, M326 W, M326H, Q327M, Q327F, Q327L, K332S, K332N, K332T, K332A, T334N, T334S, T334D, T334A, T334V, V3361, Δ338N, Δ338T, L342V, L342T, H367C, R368Y, Δ395G, P396V, P396C, V3971, V397L, Δ398C, Δ398G, L399M, L3991, L399C, T442D, T442S, V443L, V443M, a deletion at amino acid position 332, a deletion at amino acid position 335, a 5L insertion at amino acid position 327, or a combination thereof, wherein the amino acid positions correspond to SEQ ID NO:3. In some embodiments, the substitutions comprise Q275R, C285L, V323Y, E370Q, V372I, N400 W, D404A, and T438F. In some embodiments, the flavin-dependent oxidase comprises at least 70%, 80%, 90% or 95% sequence identity to SEQ ID NO:3 with substitutions Q275R, C285L, V323Y, E370Q, V372I, N400 W, D404A, T438F, L269M, I271H, Δ272C, and Δ338N. In some embodiments, the flavin-dependent oxidase comprises at least 70%, 80%, 90% or 95% sequence identity to SEQ ID NO:3 with substitutions Q275R, C285L, V323Y, E370Q, V3721, N400 W, D404A, T438F, L269M, I271H, Δ272C, Δ338N, E159A, and T442D.

In some embodiments, mutated flavin-dependent oxidase is not glycosylated.

In some embodiments, the immobilized flavin-dependent oxidase (i) does not comprise a disulfide bond (ii) is capable of oxidative cyclization of a prenylated aromatic compound into a cannabinoid, and (iii) comprises at least 70% sequence identity to any one of SEQ ID NOs:18-37.

In some embodiments, the immobilized flavin-dependent oxidase comprises at least 80% sequence identity to any one of SEQ ID NOs:18-37, or at least 90% sequence identity to any one of SEQ ID NOs:18-37.

In some embodiments, the flavin-dependent oxidase converts CBGA to cannabichromenic acid (CBCA), tetrahydrocannabinolic acid (THCA), or both. In some embodiments, the flavin-dependent oxidase converts CBGA to CBCA at about pH 4 to about pH 9. In some embodiments, the flavin-dependent oxidase converts CBGOA to cannabiorcichromenic acid (CBCOA). In some embodiments, the flavin-dependent oxidase converts CBGVA to cannabichromevarinic acid (CBCVA). In some embodiments, the flavin-dependent oxidase converts CBG to cannabichromene (CBC).

In some embodiments, the flavin-dependent oxidase converts CBGA to cannabichromenic acid (CBCA), tetrahydrocannabinolic acid (THCA), or both. In some embodiments, the flavin-dependent oxidase converts CBGA to CBCA at about pH 4 to about pH 9. In some embodiments, the flavin-dependent oxidase converts CBGOA to cannabiorcichromenic acid (CBCOA). In some embodiments, the flavin-dependent oxidase converts CBGOA to CBCOA at about pH 4 to about pH 9. In some embodiments, the flavin-dependent oxidase converts CBGVA to cannabichromevarinic acid (CBCVA). In some embodiments, the flavin-dependent oxidase converts CBGVA to CBCVA at about pH 4 to about pH 9. In some embodiments, the flavin-dependent oxidase converts CBG to cannabichromene (CBC) at about pH 4 to about pH 9.

In some embodiments, the methods used herein obtain the flavin-dependent oxidase from an engineered cell comprising the flavin-dependent oxidase, or a polynucleotide, e.g., an expression construct, or a combination thereof that can be used to produce the flavin-dependent oxidase as in known in the art.

In some embodiments, the flavin-dependent oxidase is non-natural. As described herein, a “non-natural” protein or polypeptide refers to a protein or polypeptide sequence having at least one variation at an amino acid position as compared to a wild-type polypeptide sequence. In some embodiments, the non-natural flavin-dependent oxidase has at least one variation at an amino acid position as compared to a wild-type flavin-dependent oxidase.

In some embodiments, the at least one amino acid variation comprises a substitution, deletion, insertion, or a combination thereof. In some embodiments, the variation comprises an amino acid substitution. In some embodiments, the variation comprises a deletion of one or more amino acids, e.g., about 1 to about 100, about 2 to about 80, about 5 to about 50, about 10 to about 40, about 12 to about 35, or about 14 to about 30 amino acids. In some embodiments, the variation comprises an insertion of one or more amino acids. In some embodiments, the at least one amino acid variation in the flavin-dependent oxidase is not in an active site of the flavin-dependent oxidase. In some embodiments, the active site of the flavin-dependent oxidase comprises one or more amino acid residues involved in binding the prenylated aromatic compound substrate, e.g., CBGA, CBGOA, CBGVA, CBGO, CBGV, and/or CBG. In some embodiments, the active site of the flavin-dependent oxidase comprises one or more amino acid residues involved in binding FAD cofactor. In some embodiments, the active site of the flavin-dependent oxidase comprises one or more amino acid residues involved for catalysis, e.g., the oxidative cyclization of CBGA into CBCA.

As used herein, “cannabinoid” refers to a prenylated polyketide or terpenophenolic compound derived from fatty acid or isoprenoid precursors. In general, cannabinoids are produced via a multi-step biosynthesis pathway, with the final precursor being a prenylated aromatic compound.

In some embodiments, the disclosure provides a method for producing a cannabinoid by selecting a prenylated aromatic compound of Formula I and a flavin-dependent oxidase as a catalyst for transforming the prenylated aromatic compound to a cannabinoid or a cannabinoid analog.

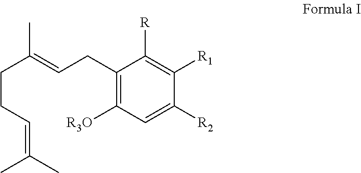

In some embodiments, R can be selected from hydroxyl (—OH), halogen, thiol (—SH), or a —NRₐR_{b} group. Substituent groups R₁ and R₂ are each independently selected from the group consisting of —H, —C(O)Rₐ, —ORₐ, an optionally substituted C₁-C₁₀ linear or branched alkylene, an optionally substituted C₂-C₁₀ linear or branched alkenylene, an optionally substituted C₂-C₁₀ linear or branched alkynylene, an optionally substituted C₃-C₁₀aryl, an optionally substituted C₃-C₁₁ cycloalkyl. (C₃-C₁₀)aryl-(C₁-C₁₀)alkylene, (C₃-C₁₀)aryl-(C₂-C₁₀)alkenylene, and (C₃-C₁₀)aryl-(C₁-C₁₀)alkynylene. Alternatively, R₁ and R₂ together with the carbon atoms to which they are bonded form a C₅-C₁₀ cyclic ring. For compounds according to Formula I, R₃ is selected from the group consisting of H, —C(O)Rₐ and C₁-C₁₀ linear or branched alkyl and Rₐ and R_{b} are each independently —H, —OH, (C₁-C₁₀) linear or branched alkyl, —SH, —NH₂, or a C₃-C₁₁ cycloalkyl.

In some embodiments, R₂ can be a linear alkylene or a branched alkylene. Exemplary of linear alkylenes include without limitation CH₃, C₂H₅, C₃H₇, C₄H₉, C₅H,,, C₆H13, C₇H₁₅ and C₈H₁₇. Illustrative of branched alkylenes are groups selected from, isopropyl, sec-butyl, iso-butyl, neopentyl, 2-methyl hexyl, or 2.3-dinethyl hexyl groups. In some embodiments. R₂ can be an optionally substituted linear or branched alkylene in which one or more hydrogen atoms is replaced without limitation with a group selected from chlorine, fluorine, bromine, nitro, amino, hydroxyl, phenyl, or benzyl group.

For certain Formula I, compounds R₂ is a C₂-C₁₀ alkenylene and is selected from the group consisting of

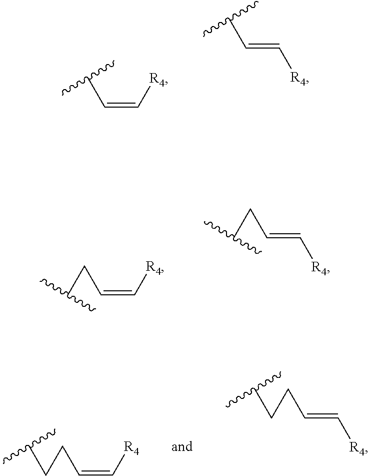

with R₄ being a linear or branched alkylene as described above. When R₂ is a C₂-C₁₀ linear or branched alkynylene, R₂ can be

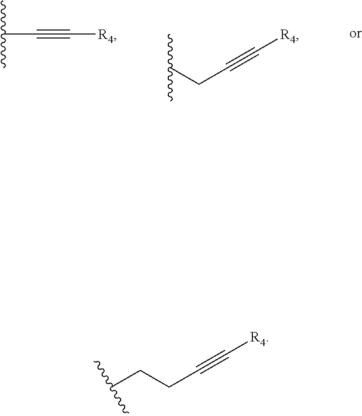

Alternatively, R2 in Formula I is

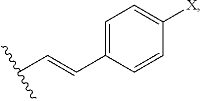

substituent X is a group selected from —OH, —SH, or NRₐR_{b} and groups Rₐ and R_{b} are as defined above.

In some embodiments, the cannabinoid obtained by contacting a prenylated aromatic compound of Formula I with a flavin-dependent oxidase can be isolated, purified and used as a therapeutic or and the cannabinoid can undergo an optional decarboxylation step to convert, for example, cannabichromenic acid (CBCA) to cannabichromene (CBC) prior to the latter's use as a pharmaceutical agent or a nutraceutical agent.

Many of the naturally occurring cannabinoids are produced as their carboxylic acid derivatives in plants. Their psychostimulatory activity is enhanced, however, following decarboxylation which occurs upon the application of heat to the cannabinoid acid containing plant tissue or by drying the plant material prior to use. Cannabinoid synthesized using the inventive method can also have a carboxylic acid (—COOH) group as the R₁ substituent and such compounds may undergo an optional decarboxylation step prior to their use as pharmaceutical or nutraceutical agents. Exemplary of such a cannabinoid is the compound obtained by contacting a Formula I species in which R is —OH, R₁ is —COOH, R₂is C₅H₁₁ and R₃ is —H with a flavin-dependent oxidase.

The term “alkyl” refers to a straight or branched chain, saturated hydrocarbon having the indicated number of carbon atoms. For example, (C₁-C₁₀)alkyl is meant to include but is not limited to methyl, ethyl, propyl, isopropyl, butyl, sec-butyl, tert-butyl, pentyl, isopentyl, neopentyl, hexyl, isohexyl, and neohexyl, etc. An alkyl group can be unsubstituted or optionally substituted with one or more substituents as described herein below.

The term “alkenyl” refers to a straight or branched chain unsaturated hydrocarbon having the indicated number of carbon atoms and at least one double bond. Examples of a (C₂-C₁₀)alkenyl group include, but are not limited to, ethylene, propylene, 1-butylene, 2-butylene, isobutylene, sec-butylene, 1-pentene, 2-pentene, isopentene. 1-hexene, 2-hexene, 3-hexene, isohexene, 1-heptene, 2-heptene, 3-heptene, isoheptene, 1-octene, 2-octene, 3-octene, 4-octene, and isooctene. An alkenyl group can be unsubstituted or optionally substituted with one or more substituents as described herein below.

The term “alkynyl” refers to a straight or branched chain unsaturated hydrocarbon having the indicated number of carbon atoms and at least one triple bond. Examples of a (C₂-C₁₀)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

The term “alkoxy” refers to an O-alkyl group having the indicated number of carbon atoms. For example, a (Cr—C₆)alkoxy group includes —O-methyl, —O-ethyl, —O-propyl, —O— isopropyl, —O-butyl, —O-sec-butyl, —O-tert-butyl, —O-pentyl, —O-isopentyl, —O-neopentyl, —O-hexyl, —O— isohexyl, and —O-neohexyl.

The term “aryl” refers to a 3- to 14-member monocyclic, bicyclic, tricyclic, or polycyclic aromatic hydrocarbon ring system. Examples of an aryl group include naphthyl. pyrenyl, and anthracyl. An aryl group can be unsubstituted or optionally substituted with one or more substituents as described herein below.

The terms “alkylene,” “alkenylene,” and “arylene,” alone or as part of another substituent, means a divalent radical derived from an alkyl, cycloalkyl, alkenyl, aryl, or heteroaryl group, respectively, as exemplified by —CH₂CH₂CH₂CH₂—. For alkylene, alkenyl, or aryl linking groups, no orientation of the linking group is implied.

The term “halogen” and “halo” refers to —F, —Cl, —Br or —I.

The term “heteroatom” is meant to include oxygen (O), nitrogen (N), and sulfur (S).

A “hydroxyl” or “hydroxy” refers to an —OH group.

The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “cycloalkyl” refer to monocyclic, bicyclic, tricyclic, or polycyclic, 3- to 14-membered ring systems, which are either saturated, unsaturated or aromatic. The heterocycle may be attached via any heteroatom or carbon atom. Cycloalkyl include aryls and hetroaryls as defined above. Representative examples of cycloalky include, but are not limited to, cycloethyl, cyclopropyl, cycloisopropyl, cyclobutyl, cyclopentyl, cyclohexyl, cyclopropene, cyclobutene. cyclopentene, cyclohexene, phenyl, naphthyl, anthracyl, benzofuranyl, and benzothiophenyl. A cycloalkyl group can be unsubstituted or optionally substituted with one or more substituents as described herein below.

The term “nitrile or cyano” can be used interchangeably and refer to a —CN group which is bound to a carbon atom of a heteroaryl ring. aryl ring and a heterocycloalkyl ring.

The term “amine or amino” refers to an —NR_{c}R_{d} group wherein R, and R_{d} each independently refer to a hydrogen, (C₁-C₅)alkyl, aryl, heteroaryl, heterocycloalkyl, (C₁-C₅)haloalkyl, and (C₁-C₆)hydroxyalkyl group.

The term “alkylaryl” refers to C₁-C₅ alkyl group in which at least one hydrogen atom of the C₁—Cs alkyl chain is replaced by an aryl atom, which may be optionally substituted with one or more substituents as described herein below. Examples of alkylaryl groups include, but are not limited to, methylphenyl, ethvlnaphthyl, propylphenyl, and butylphenyl groups.

“Arylalkylene” refers to a divalent alkylene wherein one or more hydrogen atoms in the C₁-C₁₀ alkylene group is replaced by a (C₃-C₁4)aryl group. Examples of (C₃-C₁4)aryl-(C₁-C₁₀)alkylene groups include without limitation 1-phenylbutylene, phenyl-2-butylene, 1-phenyl-2-methylpropylene, phenylmethylene, phenylpropylene, and naphthylethylene.

“Arylalkenylene” refers to a divalent alkenylene wherein one or more hydrogen atoms in the C₂-C₁₀ alkenylene group is replaced by a (C₃-C₁₄)aryl group.

The term “arylalkynylene” refers to a divalent alkynylene wherein one or more hydrogen atoms in the C₂-C₁₀ alkynylene group is replaced by a (C₃-C₁₄)aryl group.

The terms “carboxyl” and “carboxylate” include such moieties as may be represented by the general formulas:

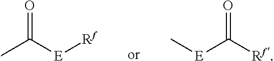

E in the formula is a bond or O and R^{f} individually is H, alkyl, alkenyl, aryl, or a pharmaceutically acceptable salt. Where E is 0, and R^{f} is as defined above, the moiety is referred to herein as a carboxyl group, and particularly when R^{f} is a hydrogen, the formula represents a “carboxylic acid”. In general, where the expressly shown oxygen is replaced by sulfur, the formula represents a “thiocarbonyl” group.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given that structure, then the depicted structure controls. Additionally, if the stereochemistry of a structure or a portion of a structure is not indicated with, for example, bold or dashed lines, the structure or portion of the structure is to be interpreted as encompassing all stereoisomers of it.

In some embodiments, the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG). In some embodiments, the prenylated aromatic compound is cannabigerolic acid (CBGA). In some embodiments, the prenylated aromatic compound is converted into a cannabinoid by oxidative cyclization. In some embodiments, the immobilized flavin-dependent oxidase converts one or more of CBGA, CBGOA, CBGVA, and CBG into a cannabinoid. In some embodiments, the immobilized flavin-dependent oxidase converts CBGA into one or more of CBCA, CBDA, or THCA. In some embodiments, the immobilized flavin-dependent oxidase converts CBGOA into one or more of CBCOA, CBDOA, or THCOA. In some embodiments, the immobilized flavin-dependent oxidase converts CBGVA into one or more of CBCVA, CBDVA, or THCVA. In some embodiments, the immobilized flavin-dependent oxidase converts CBGO into one or more of CBCO, CBDO, or THCO. In some embodiments, the immobilized flavin-dependent oxidase converts CBGO into CBCO. In some embodiments, the immobilized flavin-dependent oxidase converts CBGV into one or more of CBCV, CBDV, or THCV. In some embodiments, the immobilized flavin-dependent oxidase converts CBGV into CBCV. In some embodiments, the immobilized flavin-dependent oxidase converts CBG into one or more of CBC, CBD, or THC.

Different cannabinoids can be produced based on the way that a precursor is cyclized. For example, THCA, CBDA, and CBCA are produced by oxidative cyclization of CBGA. Examples of cannabinoids include, but are not limited to, THCA, THCV, THCO, THCVA, THCOA, THC, CBDA, CBDV, CBDO, CBDVA, CBDOA, CBD, CBCA, CBCV, CBCO, CBCVA, CBCOA, CBC, cannabinolic acid (CBNA), cannabinol (CBN), cannabicyclol (CBL), cannabivarin (CBV), cannabielsoin (CBE), cannabicitran, and isomers, analogs or derivatives thereof. As used herein, an “isomer” of a reference compound has the same molecular formula as the reference compound, but with a different arrangement of the atoms in the molecule. As used herein, an “analog” or “structural analog” of a reference compound has a similar structure as the reference compound, but differs in a certain component such as an atom, a functional group, or a substructure. An analog can be imagined to be formed from the reference compound, but not necessarily formed or derived from the reference compound. In some embodiments, analogs may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid. As used herein, a “derivative” of a reference compound is derived from a similar compound by a similar reaction. Methods of identifying isomers, analogs or derivatives of the cannabinoids described herein are known to one of ordinary skill in the art.

In some embodiments, the term “cannabinoid” includes, without limitation are CBCA, CBC, CBCOA, CBCVA, THCA, THCA, THCV, THCO, THCVA, THCOA, THC, CBDA, CBDV, CBDO, CBDVA, CBDOA, CBD, CBCA, CBCV, CBCO, CBCVA, CBCGA, CBC, cannabinolic acid (CBNA), cannabinol (CBN), cannabicyclol (CBL), cannabivarin (CBV), cannabielsoin (CBE), cannabicitran, cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Al 1-tetrahydrocannabinol, tetrahydrocannabivarin, dronabinol, amandamide and nabilone, an isomer, analog or derivative thereof, or a combination thereof, as well as natural or synthetic molecules that have a basic cannabinoid structure and are modified synthetically to provide a cannabinoid analog. In some embodiments, the cannabinoid is CBCA, CBC, CBCOA, CBCVA, or THCA. As used herein, the term “cannabinoid” includes naturally occurring cannabinoids, analogs thereof, derivatives thereof, and all isomers thereof.

In some embodiments, the disclosure provides a composition comprising a cannabinoid or an isomer, analog or derivative thereof obtained from a prokaryote or fungi cell as described herein, the cell extract described herein, or the method described herein. In some embodiments, the cannabinoid is CBCA, THCA, CBCGA, CBCVA, CBC, or an isomer, analog or derivative thereof, or a combination thereof. In some embodiments, the cannabinoid is 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition.

In some embodiments, the cannabinoid is in a therapeutic or medicinal composition. In some embodiments, the composition is a topical composition. In some embodiments, the composition is an edible composition.

In some embodiments, only a single cannabinoid is made by the flavin-dependent oxidase. In some embodiments, more than one type of cannabinoid is made by the flavin-dependent oxidase, e.g., two types of cannabinoids, three types of cannabinoids, four types of cannabinoids, etc.

In some embodiments, the flavin-dependent oxidase is derived from cloning and large scale expression of enzymes that play a role in the biosynthesis of cannabinoids. In some embodiments, the expression system is a prokaryote expression system used for the manufacture of cannabinoids. Exemplary of prokaryote cells suitable for cloning and expression of the flavin-dependent oxidase include without limitation archaea and eubacteria. Nonlimiting examples of suitable microbial hosts for the bio-production of a cannabinoid include, but are not limited to, any Gram negative organisms, more particularly a member of the family Enterobacteriaceae, such as F. coli, or Oligotropha carboxidovorans, or a Pseudomononas sp.; any Gram positive microorganism, for example Bacillus subtilis, Lactobaccilus sp. or Lactococcus sp.; a yeast, for example Saccharomyces cerevisiae, Pichia pastoris or Pichia stipitis; and other groups or microbial species. In some embodiments, the microbial host is a member of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, or Saccharomyces. In some embodiments, the microbial host is Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis or Saccharomyces cerevisiae.

Further exemplary species are reported in U.S. Pat. No. 9,657,316 and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus andAcinetobacter bavlyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkern, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicilihum chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium lot, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In some embodiments, the prokaryote cells suitable for cloning and expression of the flavin-dependent oxidase is a bacterial cell. In some embodiments. In some embodiments, the prokarvote cells suitable for cloning and expression of the flavin-dependent oxidase is an algal cell. In some embodiments, the prokaryote cells suitable for cloning and expression of the flavin-dependent oxidase is a cyanobacterial cell. In some embodiments, the bacteria is Escherichia, Corynebacterium, Bacillus, Ralstonia, Zymomonas, or Staphylococcus. In some embodiments, the bacterial cell is an Escherichia coli cell.

In some embodiments, prokaryote cells suitable for cloning and expression of the flavin-dependent oxidase is an organism selected from Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxvdothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium dfficile, Clostridium hiranonis DSM 13275, Clostridium hylemonae DSM 15053, Clostridium kluyveri, Clostridium kluyveri DSM 555, Clostridium Ijungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutvlacetonicum N1-4, Clostridium tetani. Corynebacterium glutamicum ATCC 14067, Corynebacterium glutamicum R, Corvnebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.IMC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphoera sedula, Methanosarcina acetivorans, Methanosarcina acetivorans C₂A, Methanosarcina barkeri, Methanosarcina mazei TucO1, Methylobacter marinus, Methylobacierium extorquens, Methylobacterium extorquens AM 1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nltrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PAO1, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, ihermococcus kodakaraensis, 1hermococcus litoralis, ihermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, and Zea mays. In some embodiments, the cell is a bacterial cell. In some embodiments, the cell is an E. coli cell.

In some embodiments, the expression system is a fungal expression system used for the manufacture of cannabinoids.

In some embodiments, the flavin-dependent oxidase can be expressed in an algae system. Algae that can be used for cannabinoid production include, but are not limited to, unicellular and multicellular algae. Examples of such algae can include a species of rhodophyte, chlorophyte, heterokontophyte (including diatoms), tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta.

In some embodiments, the prokaryote cells suitable for cloning and expression of the flavin-dependent oxidase is a microalgae (single-celled algae). Specific species that are considered for cannabinoid production include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditiana. Dunaliella salina. Dunaliella tertiolecta, Chlorella vulgaris, Chlorella variabilis, and Chlamydomonas reinhardti,. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrsosphaera, Cricosphaera, Crypthecodinium, Cryptomonas, Cyclotella, Dunaliella, Ellipsoidon, Emiliania. Eremosphaera, Ernodesmius, Euglena, Franceia, Fragilaria, Gloeolhamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus. Platymonas, Pleurochrsis, Pleurococcus, Prototheca, Pseudochlorella, Pyramimonas, Pvrobotrys, Scenedesmus, Skeletonema, Spyrogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylcoccopsis, Dermocarpella, Fischerella, Fremyella, Geitleria, Geitlerinema, Gloeobacter, Gloeocapsa, Gloeothece, Halospirulina, Ivengariella, Leptolyngbya, Limnothrix, Lyngbya, Microcoleus, Microcystis, Mxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirul/na, Stanieria, Starria, Stigonema, Svmploca, Synechococcus, Svnechocystis, Tlipothrix, Trichodesmium. [vchonema, andXenococcus species.

In some embodiments, the disclosure provides a cell extract or cell culture medium comprising a flavin-dependent oxidase capable of synthesizing cannabigerolic acid (CBGA), cannabichromenic acid (CBCA), tetrahydrocannabinolic acid (THCA), cannabigerol (CBG), cannabichromene (CBC), cannabigerorcinic acid (CBGOA), cannabiorcichromenic acid (CBCOA), cannabigerivarinic acid (CBGVA), cannabichromevarinic acid (CBCVA), an isomer, analog or derivative thereof, or a combination thereof. In some embodiments, the cell extract or cell culture medium is added to a reactor, wherein the flavin-dependent oxidase is immobilized on a solid support, e.g., the cell extract or cell culture medium is added to a chromatographic apparatus which selectively binds the flavin-dependent oxidase. In some embodiments, one or more impurities are removed from the cell extract or cell culture medium before the prenylated aromatic compound is added.

In some embodiments, the disclosure provides a method of making a cannabinoid selected from CBCA, CBC, CBCOA, CBCVA, THCA, an isomer, analog or derivative thereof, or a combination thereof, comprising: culturing the cell described herein expressing a flavin-dependent oxidase, separating the flavin-dependent oxidase from the intact cultured cells (e.g., via lysis or via secretion), immobilizing the flavin-dependent oxidase, and reacting the immobilized flavin-dependent oxidase with a prenylated aromatic compound, e.g., a compound of Formula I, e.g., CBGA, and then isolating the cannabinoid produced therefrom.

In some embodiments, the disclosure provides a method of making CBCA, THCA, or an isomer, analog or derivative thereof, comprising contacting CBGA with the flavin-dependent oxidase described herein. In some embodiments, the disclosure provides a method of making CBCA, THCA, or an isomer, analog or derivative thereof, or a combination thereof, comprising contacting CBGA with an immobilized flavin-dependent oxidase at least 70%, at least 80%, at least 90% or at least 95% identical to any of SEQ ID NOs:1-6 or 15-37, wherein a disulfide bond does not form. In some embodiments, the flavin-dependent oxidase is at least 70%, at least 80%, at least 90% or at least 95% identical to SEQ ID NO:1, SEQ ID NO:3, SEQ ID NO:16 or SEQ ID NO:17, wherein a disulfide bond does not form. In some embodiments, the flavin-dependent oxidase is at least 70%, at least 80%, at least 90% or at least 95% identical to SEQ ID NO:3 wherein a disulfide bond does not form.

In some embodiments, the disclosure provides a method of making CBCOA or an isomer, analog or derivative thereof, or a combination thereof, comprising contacting CBGOA with the immobilized flavin-dependent oxidase described herein. In some embodiments, the disclosure provides a method of making CBCOA or an isomer, analog or derivative thereof, or a combination thereof, comprising contacting CBGOA with an immobilized flavin-dependent oxidase at least 70%, at least 80%, at least 90% or at least 95% identical to any of SEQ ID NOs:1-6 or 15-37, wherein a disulfide bond does not form. In some embodiments, the flavin-dependent oxidase is at least 70%, at least 80%, at least 90% or at least 95% identical to SEQ ID NO:1, SEQ ID NO:3, SEQ ID NO:16 or SEQ ID NO:17, wherein a disulfide bond does not form. In some embodiments, the flavin-dependent oxidase at least 70%, at least 80%, at least 90% or at least 95% identical to SEQ ID NO:3 wherein a disulfide bond does not form.

In some embodiments, the disclosure provides a method of making CBCVA and/or an isomer, analog or derivative thereof, or a combination thereof, comprising contacting CBGVA with the immobilized flavin-dependent oxidase described herein. In some embodiments, the disclosure provides a method of making CBCVA and/or an isomer, analog or derivative thereof, or a combination thereof, comprising contacting CBGVA with the immobilized flavin-dependent oxidase at least 70%, at least 80%, at least 90% or at least 95% identical to any of SEQ ID NOs:1-6 or 15-37, wherein a disulfide bond does not form. In some embodiments, the flavin-dependent oxidase is at least 70%, at least 80%, at least 90% or at least 95% identical to SEQ ID NO:1, SEQ ID NO:3, SEQ ID NO:16 or SEQ ID NO:17, wherein a disulfide bond does not form. In some embodiments, the flavin-dependent oxidase at least 70%, at least 80%, at least 90% or at least 95% identical to SEQ ID NO:3 wherein a disulfide bond does not form.

In some embodiments, the disclosure provides a method of making CBC or an analog or derivative thereof, comprising contacting comprising contacting CBG with the immobilized flavin-dependent oxidase described herein. In some embodiments, the disclosure provides a method of making CBC or an analog or derivative thereof, comprising contacting comprising contacting CBG with an immobilized flavin-dependent oxidase at least 70%, at least 80%, at least 90% or at least 95% identical to any of SEQ ID NOs:1-6 or 15-37, wherein a disulfide bond does not form. In some embodiments, the flavin-dependent oxidase is at least 70%, at least 80%, at least 90% or at least 95% identical to SEQ ID NO:1, SEQ ID NO:3, SEQ ID NO:16 or SEQ ID NO:17, wherein a disulfide bond does not form. In some embodiments, the flavin-dependent oxidase at least 70%, at least 80%, at least 90% or at least 95% identical to SEQ ID NO:3 wherein a disulfide bond does not form.

In some embodiments, the contacting occurs at about pH 4 to about pH 9. In some embodiments, the method is performed in an in vitro reaction medium. In some embodiments, the in vitro reaction medium comprises a surfactant. In some embodiments, the surfactant is about 0.01% (v/v) to about 1% (v/v) of the in vitro reaction medium. In some embodiments, the surfactant is 2-[4-(2,4,4-trimethylpentan-2-yl)phenoxy]ethanol. In some embodiments, the in vitro reaction medium comprises about 0.1% (v/v) 2-[4-(2,4,4-trimethylpentan-2-yl)phenoxy]ethanol (Triton™ X-100).

In some embodiments, the method is performed in a chromatographic apparatus. The term “chromatographic apparatus” can include any apparatus suitable for removing and/or separating one or more impurities from the immobilized flavin-dependent oxidase. For example, in some embodiments, the chromatographic apparatus comprises a solid-support affinity matrix, wherein one or more components of a cell culture such as extracellular media comprising secreted flavin-dependent oxidase is applied to the chromatographic apparatus, whereby the flavin-dependent oxidase is retained, i.e., immobilized, on the chromatographic apparatus, and one or more cellular/media impurities is removed before the prenylated aromatic compound is provided and reacted with the flavin-dependent oxidase.

In some embodiments, the chromatographic apparatus comprises a solid-support affinity matrix, wherein one or more components of a cell culture such a lysed cellular composition comprising the flavin-dependent oxidase is applied to the chromatographic apparatus, whereby the flavin-dependent oxidase is retained, i.e., immobilized, on the chromatographic apparatus, and one or more cellular/media impurities is removed before the prenylated aromatic compound is provided and reacted with the flavin-dependent oxidase. One of skill in the art can appreciate that in some embodiments, the lysed cellular composition may undergo one or more purification or processing steps prior to being applied to the chromatographic apparatus. For examples, in some embodiments, the lysed cellular composition can be filtered, centrifuged, concentrated, etc., prior to being applied to the chromatographic apparatus.

The present disclosure provides that the synthesis, isolation and purification of cannabinoids can be improved by immobilization of a flavin-dependent oxidase lacking a disulfide bond to a solid support, or by encapsulation of the synthase within a liposome. The present invention provides that immobilizing the flavin-dependent oxidase, and then interacting the flavin-dependent oxidase with a prenylated aromatic compound unexpectedly was suitable to maintain the action of the flavin-dependent oxidase, resulting in formation of a cannabinoid.

In one aspect of the synthesis, the flavin-dependent oxidase is immobilized to a solid support. Such immobilization is advantageous, since it permits recycling and reuse of the immobilized flavin-dependent oxidase which significantly reduces the costs associated with the manufacture of pharmaceutical grade cannabinoids. Immobilization of the flavin-dependent oxidase also permits ease of use and recovery of the flavin-dependent oxidase, ease of purification of the desired cannabinoid product, preservation of the enantiomeric excess (ee) of the final product and an overall improvement in the yield of the product. In some embodiments, the enantiomeric purity of a cannabinoid according to the claimed method is from about 90% ee to about 100% ee. In some embodiments, a cannabinoid produced using the methods described herein can have an enantiomeric purity of about 91% ee, about 92% ee, about 93% ee, about 94% ee, about 95% ee, about 96% ee, about 97% ee, about 98% ee and about 99% ee.

Typically, the flavin-dependent oxidase to be immobilized can be retained on a solid support, absorbed onto a solid support, adsorbed onto a support, covalently linked to a support or can be immobilized onto a solid support through ionic interactions. One of skill in the art will appreciate that, e.g., in affinity chromatography, the flavin-dependent oxidase would be preferentially retained on the solid support matrix, while not necessarily being permanently bound to a single solid support particle, and that such equilibrium binding would be construed to be within the term “immobilized” as used herein. In one embodiment, the flavin-dependent oxidase is covalently linked to a solid support, i.e., the flavin-dependent oxidase is immobilized on a solid support by a covalent bond with the solid support. Suitable strategies for linking an enzyme to a solid support are well known in the biochemical art and include covalent linkages between an appropriately functionalized support and a side chain of an amino acid group or through covalent linkages using appropriately functionalized linkers or spacers to separate the support from the enzyme. The term “linker” refers to any group that separates the support from the enzyme. Accordingly, a linker is a group that is covalently tethered at one end to a group on the surface of the support and is attached to the enzyme at the other end. Illustrative linkers include (C₁-C₁₀)alkylene linker polymers of ethylene glycol such as a —(OCH₂—CH₂)n-—O— group, where n is an integer from 0 to 10, —(C₁-C₁₀)alkylene-NH—, —(C—C₁₀)alkylenesiloxy, or a —(C₁-C₁₀)alkylene-C(O)—.

In some embodiments, the flavin-dependent oxidase is immobilized on a solid support by an interaction with an antibody. E.g., in some embodiments, an antibody bound to the solid support is used to immobilize the flavin-dependent oxidase. In some embodiments, the flavin-dependent oxidase comprises an affinity tag. In some embodiments, the affinity tag is used to immobilize the flavin-dependent oxidase to the solid support. In some embodiments, the affinity tag used can be any affinity tag known to the skilled artisan, including, e.g., those found in Tags found in Kimple, M.E., et al., “Overview of Affinity Tags for Protein Purification” Curr. Protoc. Protein Sci. 73:9.9.1 (2018). In some embodiments, the affinity tag selected from an AUl epitope tag, an AU5 epitope tag, a bacteriophage T7 epitope tag, a bacteriophage V5 epitope tag, a bluetongue virus tag, a calmodulin binding peptide tag, a cellulose binding domain tag, a chitin binding domain tag, a E2 epitope tag, a FLAG epitope tag, a Glu-Glu (EE-tag) tag, a Human influenze hemagglutinin tag, a histidine affinity tag, a HSV epitope tag, a KT3 epitope tag, a Myc epitope tag, a PDZ ligand tag, a polyarginine tag, a polyaspartate tag, a polycysteine tag, an polyhistidine tag, a polyphenylalanine tag, a Protein C tag, an Sl tag, an S tag, a streptavadin binding peptide tag, a strep-tag, a TrpE tag, a Universal (HTTPHH) tag, or a VSV-G tag. In some embodiments, the affinity tag is a polyhistidine tag. In some embodiments, the polyhistidine tag is a 6X histidine tag.

The affinity tags can be used to immobilize any of the flavin-dependent oxidase described herein, e.g., a flavin-dependent oxidase which is at least 70%, at least 80%, at least 90% or at least 95% identical to any of SEQ ID NOs:1-6 or 15-37, wherein a disulfide bond does not form. In some embodiments, the flavin-dependent oxidase is at least 70%, at least 80%, at least 90% or at least 95% identical to SEQ ID NO:1, SEQ ID NO:3, SEQ ID NO:16 or SEQ ID NO:17, wherein a disulfide bond does not form. In some embodiments, the flavin-dependent oxidase comprising an affinity tag is at least 70%, at least 80% or at least 90% sequence identity to any of SEQ ID NO:4, SEQ ID NO:5, SEQ ID NO:6, or SEQ ID NO:15, wherein the 6x HIS affinity tag is conserved.

In some embodiments, the flavin-dependent oxidase described herein can be modified to include a secretory sequence for export of the flavin-dependent oxidase outside the cell during culturing. In some embodiments, the secretory sequence is located at the N-terminus of the flavin-dependent oxidase. In some embodiment, the secretory sequence is located at the C-terminus of the flavin-dependent oxidase.

Supports suitable for immobilizing enzymes include without limitation Amberlite resins, Duolite resins, acrylic resins such as Eupergit® C, DEAE-Sephadex and gels made using polyvinyl alcohol can be used as supports for immobilizing the flavin-dependent oxidase of the present technology.

In an embodiment of this technology, is disclosed a method for the large-scale production of several flavin-dependent oxidases, including Clz9, EncM, etc., using the prokaryote expression system. Accordingly, the large scale production of these enzymes can be carried out by transforming prokaryote with a DNA construct that comprises a gene for a flavin-dependent oxidase lacking a disulfide bond, e.g., Clz9 or EncM, and culturing the transformed prokaryote cells under conditions suitable for promoting the expression of a functionally active enzyme.

In some embodiments, the flavin-dependent oxidase can be expressed in a host cell via an exogenous gene. The term “exogenous” is intended to mean that the referenced molecule or the referenced activity is introduced into the host cell or host organism. The molecule can be introduced, for example, by introduction of an encoding nucleic acid into the host genetic material such as by integration into a host chromosome or as non-chromosomal genetic material that may be introduced on a vehicle such as a plasmid. The term “exogenous nucleic acid” means a nucleic acid that is not naturally-occurring within the host cell or host organism. Exogenous nucleic acids may be derived from or identical to a naturally-occurring nucleic acid or it may be a heterologous nucleic acid. For example, a non-natural duplication of a naturally-occurring gene is considered to be an exogenous nucleic acid sequence. An exogenous nucleic acid can be introduced in an expressible form into the host cell or host organism. The term “exogenous activity” refers to an activity that is introduced into the host cell or host organism. The source can be, for example, a homologous or heterologous encoding nucleic acid that expresses the referenced activity following introduction into the host cell or host organism.

Accordingly, the term “endogenous” refers to a referenced molecule or activity that is naturally present in the host cell or host organism. Similarly, the term when used in reference to expression of an encoding nucleic acid refers to expression of an encoding nucleic acid contained within the host cell or host organism.

The term “heterologous” refers to a molecule or activity derived from a source other than the referenced species, whereas “homologous” refers to a molecule or activity derived from the host microbial organism/species. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host cell or host organism.

It is understood that more than one exogenous nucleic acid(s) can be introduced into the host cell or host organism on separate nucleic acid molecules, on polycistronic nucleic acid molecules, or a combination thereof, and still be considered as more than one exogenous nucleic acid. For example, as disclosed herein, a host cell or host organism can be engineered to express at least two, three, four, five, six, seven, eight, nine, ten or more exogenous nucleic acids encoding a desired pathway enzyme or protein. In the case where two or more exogenous nucleic acids encoding a desired activity are introduced into a host cell or host organism, it is understood that the two or more exogenous nucleic acids can be introduced as a single nucleic acid, for example, on a single plasmid, on separate plasmids, can be integrated into the host chromosome at a single site or multiple sites, and still be considered as two or more exogenous nucleic acids. Similarly, it is understood that more than two exogenous nucleic acids can be introduced into a host cell or host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host cell or host organism.

Genes or nucleic acid sequences can be introduced stably or transiently into a host cell host cell or host organism using techniques well known in the art including, but not limited to, conjugation, clectroporation, chemical transformation, transduction, transfection, and ultrasound transformation. Optionally, for exogenous expression in E. coli or other prokaryotic host cells, some nucleic acid sequences in the genes or cDNAs of eukaryotic nucleic acids can encode targeting signals such as an N-terminal mitochondrial or other targeting signal, which can be removed before transformation into the prokaryotic host cells, if desired. For example, removal of a mitochondrial leader sequence led to increased expression in E. coli (Hoffmeister et al. (2005), J Biol Chem 280: 4329-4338). For exogenous expression in yeast or other eukaryotic host cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques known in the art to achieve optimized expression of the proteins.

In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Computer algorithms for codon optimizing a particular sequence for expression in a particular host cell are available and include, e.g., Integrated DNA Technologies' Codon Optimization tool, Entelechon's Codon Usage Table Analysis Tool, GenScript's OptimumGene tool, and the like. In some embodiments, the disclosure provides codon optimized polynucleotides expressing a flavin-dependent oxidase or variant thereof.

Cannabinoid synthase enzymes isolated from C. sativa have been known to at times produce more than one cannabinoid. E.g., a single synthase from C. sativa may produce tetrahydrocannabinolic acid (THCA) and cannabichromenic acid (CBCA) using the same substrate cannabigerolic acid (CBGA). In some embodiments, this is dependent on the conditions under which the cyclization reaction is catalyzed. In some embodiments, the methods described herein provide for production of a single cannabinoid using the described flavin-dependent oxidases, i.e., the flavin-dependent oxidase is highly specific to the cannabinoid produced.

In some embodiments, conditions of the present invention can be modified in the reaction mixture such that the flavin-dependent oxidase produces more than one cannabinoid. In some embodiments, temperature, pH, solvent, ionic strength and incubation times can be modified to produce one or more cannabinoids. In some embodiments, the reaction mixture comprises a solvent. In one embodiment, the type of solvent used and its concentration can alter then amount and/or types of cannabinoids produced.

Cannabinoids are lipophilic in nature and in some embodiments are poorly solubilized in aqueous solvents. The present disclosure provides that in some embodiments, a flavin-dependent oxidase can retain its catalytic activity in a solvent mixture containing buffer and a non-aqueous solvent, such as dimethyl sulfoxide (DMSO), dimethyl formamide (DMF), iso-propyl alcohol, ethanol and cyclodextrin. In some embodiments, the amount of the solvent in the reaction mixture is between 5% and 30% (w/v), about 5% to about 25%, about 5% to about 20%, about 5% to about 15% or about 5% to about 10%. In some embodiments, the amount of the solvent in the reaction mixture is between 10% and 30% (w/v), about 10% to about 25%, about 10% to about 20%, or about 10% to about 15%. In some embodiments, the flavin-dependent oxidase can retain its catalytic activity in a solvent mixture if the concentration of non-aqueous solvent in the mixture was maintained below 40% v/v, below 30%, or below 20%.

In some embodiments, the reaction mixture further comprises a solubility additive, e.g., that aids in solubilizing the cannabinoids described herein. In some embodiments, the reaction mixture comprises a solubility additive. In some embodiments, the solubility additive comprises a surfactant. In some embodiments, the solubility additive comprises a nonionic surfactant. In some embodiments, the nonionic surfactant comprises a polysorbate, e.g., polysorbate 20 (TWEEN® 20) or polysorbate 80 (TWEEN® 80), or polyethylene glycol tert-octylphenyl ether, also known as TRITONT^{M} X-100.

In some embodiments, flavin-dependent oxidase catalysis can be effective when the concentration of the non-aqueous solvent was about 20%. In some embodiments, different ratios of types of cannabinoids produced can be achieved by lowering the non-aqueous concentration, e.g., to less than 18%, less than 16%, less than 14%, less than 12%, less than 10%, less than 8%, less than 6% or less than 4%. For example, the ratio of THCA:CBCA, THCA:CBDA, or CBDA:CBCA can be altered by changing the amount of non-aqueous solvent. In some embodiments, the ratio of THCA:CBCA, THCA:CBDA, or CBDA:CBCA can range from 100:0 to 0:100, 100:1 to 1:100, 20:1 to 1:20, 10:1 to 1:10, 5: to 1:5, 1:2 to 2:1, or 1:1.

In some embodiments, different ratios of types of cannabinoids produced can be achieved by altering the pH of the reaction mixture. In some embodiments, the pH of the reaction mixture can be about 4 to about 9, about 5 to about 8, about 6 to about 8 or about 7. In some embodiments, the reaction mixture has a pH of about 4.0 to about 8.0. For example, the ratio of THCA:CBCA, THCA:CBDA, or CBDA:CBCA can be altered by changing the pH of the reaction mixture. In some embodiments, the ratio of THCA:CBCA, THCA:CBDA, or CBDA:CBCA can range from 100:0 to 0:100, 100:1 to 1:100, 20:1 to 1:20, 10:1 to 1:10, 5:1 to 1:5, 1:2 to 2:1, or 1:1 depending on the pH of the reaction mixture.

Any physical property known to have an effect on enzyme activity and catalysis can be modulated to alter the ratio of the cannabinoid products. In one embodiment, therefore, the pH of the reaction mixture was changed to modulate the ratio of THCA to CBCA produced enzymatically as products. In some embodiments, catalysis at a lower pH in the range from about 4.0 to about 6.0 favored the formation of one cannabinoid (e.g., THCA) produced by the flavin-dependent oxidase, while catalysis at a neural pH in the range from about 6.5 to about 7.5 favored the formation of a different cannabinoid (e.g., CBCA).

Thus, the present disclosure provides that that it is possible to control the formation of a type of cannabinoid, e.g., THCA or CBCA as the product of a flavin-dependent oxidase catalysis by controlling the pH of the reaction mixture.

In some embodiments, other physical properties such as the compositional make-up of the reaction solvent, ionic strength. temperature, pressure, viscosity of the reaction medium and concentration of reagents can also alter product ratio.

In some embodiments, the cannabinoid can be prepared using bioreactors and chromatographic apparatuses known to those in the art. See, e.g., U.S. Pat. No. 9,359,625 which is incorporated herein by reference in its entirety. Thus, in one embodiment is provided a system for producing a cannabinoid or a cannabinoid analog by controlling a condition that influences the quantity of a first cannabinoid or its analog formed in relation to the quantity of a second cannabinoid or its analog. The system may comprise a fermenter, a filter, a bioreactor, and optionally a control mechanism.

In some embodiments, the fermenter holds cell culture medium and a plurality of cells. The cells are configured to produce (and in some options secrete) a flavin-dependent oxidase. The cells used in the fermenter for the manufacture of a flavin-dependent oxidase can be any prokaryote cell (or in some embodiments, fungal cells) that has been genetically modified to include a nucleic acid sequence or a gene that encodes a flavin-dependent oxidase. In certain embodiments, the nucleic acid sequence that encodes a flavin-dependent oxidase is modified to include a secretion sequence at its 5′ end or 3′ end. In some embodiments, the nucleic acid sequence that encodes a flavin-dependent oxidase is modified to include and to incorporate an affinity tag, e.g., a 6-residue histidine tag at its 3′ end or 5′ end. In some embodiments, the addition of the secretion sequence permits secretion of the flavin-dependent oxidase into the medium used for prokaryote cell growth. In some embodiments, the extracellular secretion of the flavin-dependent oxidase is advantageous, since it facilitates the separation and transport of the enzyme between the fermenter and the bioreactor using the filter. Following production of flavin-dependent oxidase in the fermenter, the supernatant (e.g., medium, cells, and flavin-dependent oxidase) is transported along a path to a filter. The path may be a pipe or any other pathway suitable for transporting the supernatant.

In some embodiments, no secretion sequence is found on the flavin-dependent oxidase and the flavin-dependent oxidase would remain in the cytoplasm. In those instances, the cells can be harvested, lysed (e.g., chemically or physically lysed), and then transported to the filter.

The filter may filter the supernatant (or lysed cells) to at least partially separate the cell particles/debris from the medium containing the expressed flavin-dependent oxidase. In some embodiments, the filter can separate at least 50%, at least 60%, at least 70% or at least 80% of the total cell particles/debris from the medium. For certain embodiments, the filter separates at least 85%, at least 90%, at least 91%, at least 92%, at least 93%, at least 94%, at least 95%, at least 96%, at least 97%, at least 98%, at least 99%, or 100% of the total cell particles/debris from the medium comprising the flavin-dependent oxidase prior to the introduction of this medium into the bioreactor. In some embodiments, following filtration, the cells are transported back to the fermenter along a separate path. In one embodiment, the filter can be a filtration and purification system that includes multiple filters and reservoirs to purify the flavin-dependent oxidase.

After passing through the filter, the flavin-dependent oxidase flows into the bioreactor along a path and enters the bioreactor through an inlet. In some embodiments, the bioreactor is a chromatographic apparatus. The bioreactor can also include an inlet for reactants, such as the prenylated aromatic compound, e.g., CBGA, or other substrates according to the Formula I compound described above. In some embodiments, the biorcactor contains a reactant that is configured to interact with the flavin-dependent oxidase to form a cannabinoid. In some embodiments, the bioreactor may also provide the enviromnent for synthesis of a second cannabinoid. In some embodiments, the second cannabinoid may be produced using the same type of flavin-dependent oxidase and prenylated aromatic compound substrate as the first cannabinoid. For example, both the first cannabinoid and the second cannabinoid may be produced using CBGA as reactant and Clz9 as the flavin-dependent oxidase. By way of example, the first cannabinoid may be THCA and the second cannabinoid may be CBCA. In an alternative embodiment, the second cannabinoid may be synthesized using a prenylated aromatic compound substrate or flavin-dependent oxidase different from those used in synthesis of the first cannabinoid.

In some embodiments, the bioreactor can be a column bioreactor having a solid support that is impregnated with divalent metal ions or a support whose surface is functionalized with divalent metal ions. Typically, sepharose, agarose or other biopolymers are used as supports for binding divalent metal ions such as nickel, cobalt, magnesium and manganese. Such supports have a strong affinity for the histidine tag that can be present on the expressed flavin-dependent oxidase and can be used to sequester the flavin-dependent oxidase and separate it from other non-essential proteins and debris that may interfere or impede camiabinoid synthesis.

In some embodiments, the bioreactor used for synthesizing cannabinoids is configured for batch and continuous synthetic processes to permit commercial production of pharmaceutically useful cannabinoids. In one embodiment, the bioreactor is configured for batch synthesis in which the composition of the medium, concentration of the flavin-dependent oxidase and prenylated aromatic compound substrate are fixed at the beginning of the process and not allowed to change during catalysis. Synthesis is terminated when the concentration of the desired product in the medium of the bioreactor reaches a predetennined value or the concentration of prenylated aromatic compound substrate falls below a predetermined level, such as to a level where there is no detectable catalytic conversion of substrate to product.

In one embodiment, therefore, a His-tagged flavin-dependent oxidase is sequestered onto a nickel containing resin support within the bioreactor column prior to the introduction of a known amount of substrate, for example, cannabigerolic acid (CBGA), or a substrate of Formulae I, into the bioreactor. In an alternate embodiment, cannabigerolic acid (CBGA), or substrate of Formulae I can be present within the bioreactor having a nickel resin support prior to the introduction of the medium containing the flavin-dependent oxidase into the bioreactor. In either case, a known amount of the flavin-dependent oxidase is contacted with a known amount of substrate to synthesize a cannabinoid as product, such as the first cannabinoid or the second cannabinoid.

The progress of the reaction within the bioreactor can be monitored periodically or continuously. For instance, an optical monitoring system may be utilized to detect the concentration of product in the medium within the bioreactor as a function of time. Alternatively, the decrease in the concentration of substrate can be monitored to signal termination of synthesis. The cannabinoid product thus produced can be readily recovered from the medium using standard solvent extraction or chromatographic purification methods. The monitoring system may be part of or may interact with the control mechanism, described further below.

An alternative to the batch process mode is the continuous process mode in which a defined amount of prenylated aromatic compound substrate and medium are continuously added to the bioreactor while an equal amount of medium containing the cannabinoid product is simultaneously removed from the bioreactor to maintain a constant rate for formation of product. Medium can enter the bioreactor through and inlet and exit the bioreactor through an outlet. Methods of modulating the concentration of prenylated aromatic compound substrate, flavin-dependent oxidase and other factors implicated to maximize the rate of product formation are known in the art.

The conditions of the bioreactor can be controlled using a control mechanism. The control mechanism may be coupled to the bioreactor or, alternatively, may interact with the bioreactor wirelessly or remotely. The control mechanism can control at least one condition of the bioreactor so as to influence a quantity formed of the first cannabinoid, and optionally relative to a quantity formed of a second cannabinoid. For example, in one embodiment, the flavin-dependent oxidase is Clz9 produced by E. coli cells. As described above, in some embodiments, contact of flavin-dependent oxidase with cannabigerolic acid pennits the production of CBCA, or alternatively, both THCA and CBCA. In some embodiments, one condition that may influence the quantity of THCA produced relative to CBCA (e.g., the ratio of THCA to CBCA) is the pH of the medium in the bioreactor. Other conditions within the bioreactor may also influence the relative quantities of a first cannabinoid (e.g., THCA) and second cannabinoid (e.g., CBCA) produced in the bioreactor, such as temperature, pressure, and flow rate. In one embodiment, a change in condition (e.g., pH, temperature, pressure, and/or flow rate) can cause a shift from formation of the first cannabinoid in greater quantities relative to the second cannabinoid to formation of the second cannabinoid in greater quantities relative to the first cannabinoid.

In another embodiment, the control mechanism can also be used to control the conditions of the fermenter, such the oxygen level, agitation, pH, and feed rate. The control mechanism may also control the flow of materials (e.g., by controlling pumps) into and out of the fermenter, filter, and bioreactor.

The control mechanism can include a processing circuit having a processor and memory device. The processor can be implemented as a general purpose processor, an application specific integrated circuit (ASIC), one or more field programmable gate arrays (FPGAs), a group of processing components, or other suitable electronic processing components. The memory device (e.g., memory, memory unit, storage device, etc.) is one or more devices (e.g., RAM, ROM, Flash memory, hard disk storage, etc.) for storing data and/or computer code for completing or facilitating the various processes and functions described in the present application, such as controlling the pH, temperature, and pressure of the bioreactor, or altering the flow rate of medium into or out of the bioreactor. The memory device may be or include volatile memory or non-volatile memory. The memory device may include database components, object code components, script components, or any other type of information structure for supporting the various activities and information structures described in the present application. According to one embodiment, the memory device is communicably connected to the processor via the processing circuit and includes computer code for executing (e.g., by the processing circuit and/or processor) one or more processes described herein.

The present disclosure contemplates methods, systems and program products on any machine-readable media for accomplishing various operations, such as controlling the conditions of the bioreactor. The embodiments of the present disclosure may be implemented using existing computer processors, or by a special purpose computer processor for an appropriate system, incorporated for this or another purpose, or by a hardwired system.

The control mechanism may further include additional devices, such as a keyboard and display, to allow a user to interact with the control mechanism to control the conditions of bioreactor. For example, the display may include a screen to allow a user to monitor changes in pH, temperature, pressure, and flow rate of the bioreactor, or to monitor any other condition of the system for producing cannabinoids or cannabinoid analogs.

The construction and arrangement of the system for producing cannabinoids or cannabinoid analogs as shown in the various exemplary embodiments are illustrative only. Although only a few embodiments have been described in detail in this disclosure, many modifications are possible (e.g., variations in sizes, dimensions, structures, shapes and proportions of the various elements, values of parameters, use of materials, colors, orientations, etc.). For example, the position of elements may be reversed or otherwise varied and the nature or number of discrete elements or positions may be altered or varied. Accordingly, all such modifications are intended to be included within the scope of the present disclosure. Furthermore, the order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

The present technology is further described by the following examples which are not meant to limit the scope of the claims.

Sequences
Wild-type EncM
SEQ ID NO: 1
MQFPQLDPATLAAFSAAFRGELIWPSDADYDEARRIWNGTIDRRPALIARCTSTPDVVAAVSFARKSGLLVAVRGGGHSMAGHSVC
DGGIVIDLSLMNSIKVSRRLRRARAQGGCLLGAFDTATQAHMLATPAGVVSHTGLGGLVLGGGFGWLSRKYGLSIDNLTSVEIVTA
DGGVLTASDTENPDLFWAVRGGGGNFGVVTAFEFDLHRVGPVRFASTYYSLDEGPQVIRAWRDHMATAPDELTWALYLRLAPPLPE
LPADMHGKPVICAMSCWIGDPHEGERQLESILHAGKPHGLTKATLPYRALQAYSFPGAVVPDRIYTKSGYLNELSDEATDTVLEHA
ADIASPFTQLELLYLGGAVARVPDDATAYPNRQSPFVTNLAAAWMDPTEDARHTAWAREGYRALAGHLSGGYVNFMNPGEADRTRE
AYGAAKFERLQGVKAKYDPTNLFRLNQNIPPS
EncM T139V
SEQ ID NO: 2
MQFPQLDPATLAAFSAAFRGELIWPSDADYDEARRIWNGTIDRRPALIARCTSTPDVVAAVSFARKSGLLVAVRGGGHSMAGHSVC
DGGIVIDLSLMNSIKVSRRLRRARAQGGCLLGAFDTATQAHMLATPAGVVSHVGLGGLVLGGGFGWLSRKYGLSIDNLTSVEIVTA
DGGVLTASDTENPDLFWAVRGGGGNFGVVTAFEFDLHRVGPVRFASTYYSLDEGPQVIRAWRDHMATAPDELTWALYLRLAPPLPE
LPADMHGKPVICAMSCWIGDPHEGERQLESILHAGKPHGLTKATLPYRALQAYSFPGAVVPDRIYTKSGYLNELSDEATDTVLEHA
ADIASPFTQLELLYLGGAVARVPDDATAYPNRQSPFVTNLAAAWMDPTEDARHTAWAREGYRALAGHLSGGYVNFMNPGEADRTRE
AYGAAKFERLQGVKAKYDPTNLFRLNQNIPPS
Wild-type Clz9
SEQ ID NO: 3
MTADPSSERSDMNEADEVNEVDELSETGQTSGTKGKRPFTGRVIGPADGEFDEARRVWNECFAARPKEIVYCADTRDVVRALREVR
QRGGPFRVRSGGHSMSGLSVLDDGTVLDVSGLDDIQVSEDASTVTVGSGAHLGDIFRALWARGVTVPAGFCPEIGIAGHVLGGGAG
ILVRSRGFLSDHLVALEMVDSEGRIVVADHDSHHELLWASRGGGGGNFGIATSFTLRTQPIGDVTLFTIAWDWDRGAEAIKAWQEW
LATADGRINTLFIAYPQDQDMFAALGCFEGDAAELEPLIAPLVHAVEPTEKVAETMPWIEALSFVETMQGEAMKATSVRAKGNLSF
VTEPLGDRAVEEIKKALAQAPSHRAEVVLYGLGGAVAAKGRRETAFVHRDAPVALNYHTDWDDEAEDDLNFAWIQNLRASVAAHTE
GRGSYVNTIDLTVEHWLWDYYEENLPRLMAVKKRYDPEDVFRHPQSIPVSLTEAEAAELGIPPHIAEELRAARQLR
EncM with N-terminal 6xHis tag and thrombin cleavage site
SEQ ID NO: 4
MGSSHHHHHHSSGLVPRGSQFPQLDPATLAAFSAAFRGELIWPSDADYDEARRIWNGTIDRRPALIARCTSTPDVVAAVSFARKSG
LLVAVRGGGHSMAGHSVCDGGIVIDLSLMNSIKVSRRLRRARAQGGCLLGAFDTATQAHMLATPAGVVSHTGLGGLVLGGGFGWLS
RKYGLSIDNLTSVEIVTADGGVLTASDTENPDLFWAVRGGGGNFGVVTAFEFDLHRVGPVRFASTYYSLDEGPQVIRAWRDHMATA
PDELTWALYLRLAPPLPELPADMHGKPVICAMSCWIGDPHEGERQLESILHAGKPHGLTKATLPYRALQAYSFPGAVVPDRIYTKS
GYLNELSDEATDTVLEHAADIASPFTQLELLYLGGAVARVPDDATAYPNRQSPFVTNLAAAWMDPTEDARHTAWAREGYRALAGHL
SGGYVNFMNPGEADRTREAYGAAKFERLQGVKAKYDPTNLFRLNQNIPPS
EncM T139V with N-terminal 6xHis tag and thrombin cleavage site
SEQ ID NO: 5
MGSSHHHHHHSSGLVPRGSQFPQLDPATLAAFSAAFRGELIWPSDADYDEARRIWNGTIDRRPALIARCTSTPDVVAAVSFARKSG
LLVAVRGGGHSMAGHSVCDGGIVIDLSLMNSIKVSRRLRRARAQGGCLLGAFDTATQAHMLATPAGVVSHVGLGGLVLGGGFGWLS
RKYGLSIDNLTSVEIVTADGGVLTASDTENPDLFWAVRGGGGNFGVVTAFEFDLHRVGPVRFASTYYSLDEGPQVIRAWRDHMATA
PDELTWALYLRLAPPLPELPADMHGKPVICAMSCWIGDPHEGERQLESILHAGKPHGLTKATLPYRALQAYSFPGAVVPDRIYTKS
GYLNELSDEATDTVLEHAADIASPFTQLELLYLGGAVARVPDDATAYPNRQSPFVTNLAAAWMDPTEDARHTAWAREGYRALAGHL
SGGYVNFMNPGEADRTREAYGAAKFERLQGVKAKYDPTNLFRLNQNIPPS
Clz9 with N-terminal MBP tag
SEQ ID NO: 6
MGSSHHHHHHGSSGASEEGKLVIWINGDKGYNGLAEVGKKFEKDTGIKVTVEHPDKLEEKFPQVAATGDGPDIIFWAHDRFGGYAQ
SGLLAEITPDKAFQDKLYPFTWDAVRYNGKLIAYPIAVEALSLIYNKDLLPNPPKTWEEIPALDKELKAKGKSALMFNLQEPYFTW
PLIAADGGYAFKYENGKYDIKDVGVDNAGAKAGLTFLVDLIKNKHMNADTDYSIAEAAFNKGETAMTINGPWAWSNIDTSKVNYGV
TVLPTFKGQPSKPFVGVLSAGINAASPNKELAKEFLENYLLTDEGLEAVNKDKPLGAVALKSYEEELAKDPRIAATMENAQKGEIM
PNIPQMSAFWYAVRTAVINAASGRQTVDEALKDAQTNSITSLYGSSGSSGSNLYFQSNGMTADPSSERSDMNEADEVNEVDELSET
GQTSGTKGKRPFTGRVIGPADGEFDEARRVWNECFAARPKEIVYCADTRDVVRALREVRQRGGPFRVRSGGHSMSGLSVLDDGTVL
DVSGLDDIQVSEDASTVTVGSGAHLGDIFRALWARGVTVPAGFCPEIGIAGHVLGGGAGILVRSRGFLSDHLVALEMVDSEGRIVV
ADHDSHHELLWASRGGGGGNFGIATSFTLRTQPIGDVTLFTIAWDWDRGAEAIKAWQEWLATADGRINTLFIAYPQDQDMFAALGC
FEGDAAELEPLIAPLVHAVEPTEKVAETMPWIEALSFVETMQGEAMKATSVRAKGNLSFVTEPSGDRAVEEIKKALAQAPSHRAEV
VLYGLGGAVAAKGRRETAFVHRDAPVALNYHTDWDDEAEDDLNFAWIQNLRASVAAHTEGRGSYVNTIDLTVEHWLWDYYEENLPR
LMAVKKRYDPEDVFRHPQSIPVSLTEAEAAELGIPPHIAEELRAARQLR
UniProt KB A0A2E0XWX6 from Phycisphaerae bacterium with C-terminal
linker and 6xHis tag
SEQ ID NO: 7
MQACNNHQLTDAILQEFSQTLSGDLVLPTDGLYQWARLIHHTNFDGIYPVGIVFCETPEDVSKAILFARQFGLHVTARGGGHSYEG
YSVTGGLLIDVSRMNSVSVNPAAMTAVVGAGAVLIDVYHGLYYPHKLSIPGGSCPSVGIAGYLLGGGVGLQSRTYGVGCDRVLEIG
VVLASGEYVVASPTNHSDLYWAYRGGGGGNFGVVTHFKMQCHPVDRLSYAIITWDWEAAAPAFNAWQNWLKGLGEDYRNFSIFKFL
VNAGGDGGLGPKVNLIVQFDSQSNTGTGELETLMAPLLNTAHEHIVSQTIMNGDFFEVTMEIMAGCAWPKTDLDTEFLRCHTVGNP
AFPMASLPRDTYKAKSTFFADVISEKGIETCIKAIEDRFNSNLPDNSSQFTCALQFDSEGGIMGDVPKDATAFMHRDCLMHCQYLA
YWPAEGDAWFDDNPDYSYCDVSNGSMQWISDAFNVLWPFGNGHAYQNYIDKEQPNWLYAYYGENVERLRTVKAKYDPDNIWKFEQS
IPPAQGGSGGSGSGSGGSGSHHHHHH
TamL from Streptomyces sp. 307-9 with N-terminal 6xHis tag and thrombin
cleavage site
SEQ ID NO: 8
MGSSHHHHHHSSGLVPRGSKHIDSVAPGDIRYEDLRRGENLRFVGDPEEIHLVGSAAEIEQVLSRAVRSGKRVAVRSGGHCYEDFV
ANSDVRVVMDMSRLSAVGFDEERGAFAVEAGATLGAVYKTLFRVWGVTLPGGACPDVGAGGHILGGGYGPLSRMHGSIVDYLHAVE
VVVVDASGDARTVIATREPSDPNHDLWWAHTGGGGGNFGVVVRYWLRTAEADVPPEPGRLLPRPPAEVLLNTTVWPWEGLDEAAFA
RLVRNHGRWFEQNSGPDSPWCDLYSVLALTRSQSGALAMTTQLDATGPDAEKRLETYLAAVSEGVGVQPHSDTRRLPWLHSTRWPG
IAGDGDMTGRAKIKAAYARRSFDDRQIGTLYTRLTSTDYDNPAGVVALIAYGGKVNAVPADRTAVAQRDSILKIVYVTTWEDPAQD
PVHVRWIRELYRDVYADTGGVPVPGGAADGAYVNYPDVDLADEEWNTSGVPWSELYYKDAYPRLQAVKARWDPRNVFRHALSVRVP
PA
Polynucleotide encoding SEQ ID NO: 4
SEQ ID NO: 9
ATGGGCAGCAGCCATCATCATCATCATCACAGCAGCGGCCTGGTGCCGCGCGGCAGCCAGTTCCCGCAGTTGGACCCTGCTACTTT
AGCCGCGTTCTCCGCAGCTTTCCGCGGCGAACTGATTTGGCCATCCGATGCAGACTACGATGAAGCGCGCCGTATCTGGAACGGTA
CTATCGACCGCCGTCCGGCTCTCATCGCGCGCTGTACTAGCACCCCGGACGTGGTTGCTGCGGTTTCCTTCGCGCGCAAATCTGGC
CTGCTTGTGGCGGTTCGTGGCGGTGGCCACTCTATGGCTGGTCACTCGGTATGCGACGGTGGCATTGTGATTGACCTGTCTCTGAT
GAACTCCATCAAAGTATCCCGCCGTCTGCGCCGTGCTCGTGCGCAGGGTGGCTGCCTGCTCGGTGCTTTCGACACTGCTACCCAGG
CGCATATGCTCGCGACCCCGGCTGGCGTCGTTTCCCACACCGGCCTGGGCGGTCTGGTTCTGGGTGGCGGTTTCGGCTGGCTGTCA
CGTAAATATGGCCTGAGCATCGACAACCTGACCTCTGTAGAAATCGTGACCGCTGACGGTGGCGTGCTGACCGCATCCGATACTGA
GAACCCGGACTTATTCTGGGCTGTTCGCGGAGGAGGAGGTAATTTTGGCGTCGTAACCGCTTTCGAATTCGACTTACACCGCGTCG
GCCCAGTTCGTTTCGCATCGACCTACTATAGCCTCGATGAAGGCCCGCAGGTCATCCGTGCTTGGCGTGACCACATGGCGACGGCA
CCGGATGAACTGACCTGGGCGCTCTATCTGCGTCTGGCGCCGCCACTGCCGGAACTGCCTGCAGACATGCACGGCAAACCGGTTAT
CTGCGCAATGTCTTGCTGGATTGGTGACCCACATGAAGGTGAACGTCAGTTAGAATCTATTCTGCATGCCGGTAAACCGCACGGCC
TGACTAAAGCGACCCTTCCGTACCGCGCACTGCAGGCCTATTCCTTCCCGGGCGCAGTCGTTCCGGACCGTATCTACACTAAATCC
GGGTACCTGAATGAGCTGTCCGACGAGGCGACCGATACCGTGCTTGAACACGCAGCGGATATCGCGTCGCCGTTCACTCAACTTGA
ACTGCTCTACCTGGGCGGTGCCGTTGCTCGTGTTCCAGATGACGCGACCGCTTATCCTAACCGCCAGTCTCCGTTTGTGACCAACC
TGGCGGCAGCTTGGATGGATCCGACCGAGGATGCTCGTCACACCGCTTGGGCGCGCGAAGGTTACCGTGCGCTGGCGGGCCATCTG
TCCGGTGGCTACGTTAACTTTATGAACCCGGGTGAGGCGGACCGTACCCGTGAAGCCTACGGCGCGGCAAAATTTGAGCGCCTGCA
GGGCGTGAAAGCTAAATATGACCCGACTAACCTGTTCCGTCTGAATCAGAACATTCCGCCATCCTAG
Polynucleotide encoding SEQ ID NO: 5
SEQ ID NO: 10
ATGGGCAGCAGCCATCATCATCATCATCACAGCAGCGGCCTGGTGCCGCGCGGCAGCCAGTTCCCGCAGTTGGACCCTGCTACTTT
AGCCGCGTTCTCCGCAGCTTTCCGCGGCGAACTGATTTGGCCATCCGATGCAGACTACGATGAAGCGCGCCGTATCTGGAACGGTA
CTATCGACCGCCGTCCGGCTCTCATCGCGCGCTGTACTAGCACCCCGGACGTGGTTGCTGCGGTTTCCTTCGCGCGCAAATCTGGC
CTGCTTGTGGCGGTTCGTGGCGGTGGCCACTCTATGGCTGGTCACTCGGTATGCGACGGTGGCATTGTGATTGACCTGTCTCTGAT
GAACTCCATCAAAGTATCCCGCCGTCTGCGCCGTGCTCGTGCGCAGGGTGGCTGCCTGCTCGGTGCTTTCGACACTGCTACCCAGG
CGCATATGCTCGCGACCCCGGCTGGCGTCGTTTCCCACGTGGGCCTGGGCGGTCTGGTTCTGGGTGGCGGTTTCGGCTGGCTGTCA
CGTAAATATGGCCTGAGCATCGACAACCTGACCTCTGTAGAAATCGTGACCGCTGACGGTGGCGTGCTGACCGCATCCGATACTGA
GAACCCGGACTTATTCTGGGCTGTTCGCGGAGGAGGAGGTAATTTTGGCGTCGTAACCGCTTTCGAATTCGACTTACACCGCGTCG
GCCCAGTTCGTTTCGCATCGACCTACTATAGCCTCGATGAAGGCCCGCAGGTCATCCGTGCTTGGCGTGACCACATGGCGACGGCA
CCGGATGAACTGACCTGGGCGCTCTATCTGCGTCTGGCGCCGCCACTGCCGGAACTGCCTGCAGACATGCACGGCAAACCGGTTAT
CTGCGCAATGTCTTGCTGGATTGGTGACCCACATGAAGGTGAACGTCAGTTAGAATCTATTCTGCATGCCGGTAAACCGCACGGCC
TGACTAAAGCGACCCTTCCGTACCGCGCACTGCAGGCCTATTCCTTCCCGGGCGCAGTCGTTCCGGACCGTATCTACACTAAATCC
GGGTACCTGAATGAGCTGTCCGACGAGGCGACCGATACCGTGCTTGAACACGCAGCGGATATCGCGTCGCCGTTCACTCAACTTGA
ACTCCTCTACCTCCCCCCTCCCCTTGCTCCTCTTCCAGATGACCCGACCCCTTATCCTAACCCCCACTCTCCCTTTCTCACCAACC
TGGCGGCAGCTTGGATGGATCCGACCGAGGATGCTCGTCACACCGCTTGGGCGCGCGAAGGTTACCGTGCGCTGGCGGGCCATCTG
TCCGGTGGCTACGTTAACTTTATGAACCCGGGTGAGGCGGACCGTACCCGTGAAGCCTACGGCGCGGCAAAATTTGAGCGCCTGCA
GGGCGTGAAAGCTAAATATGACCCGACTAACCTGTTCCGTCTGAATCAGAACATTCCGCCATCCTAG
Polynucleotide encoding SEQ ID NO: 3
SEQ ID NO: 11
ATGACCGCAGATCCGAGCAGCGAACGTAGCGATATGAATGAAGCAGATGAAGTGAACGAAGTTGATGAACTGAGCGAAACCGGTCA
GACCAGCGGCACCAAAGGTAAACGTCCGTTTACAGGTCGTGTTATTGGTCCGGCAGATGGTGAATTTGATGAAGCACGTCGTGTTT
GGAATGAATGTTTTGCAGCACGTCCGAAAGAAATTGTTTATTGTGCAGATACCCGTGATGTTGTTCGTGCACTGCGTGAAGTTCGT
CAGCGTGGTGGTCCGTTTCGTGTTCGTAGCGGTGGTCATAGCATGAGCGGTCTGAGCGTTCTGGATGATGGTACAGTGCTGGATGT
TAGTGGCCTGGATGATATTCAGGTTAGCGAAGATGCAAGCACCGTTACCGTTGGTAGCGGTGCACATCTGGGTGATATTTTTCGTG
CCCTGTGGGCACGTGGTGTTACCGTTCCGGCAGGTTTTTGTCCGGAAATTGGTATTGCAGGTCATGTTTTAGGTGGTGGTGCAGGT
ATTCTGGTGCGTAGCCGTGGTTTTCTGAGCGATCATCTGGTTGCACTGGAAATGGTTGATAGCGAAGGTCGTATTGTTGTTGCAGA
TCATGATAGTCATCATGAACTGCTGTGGGCAAGCCGTGGTGGTGGCGGTGGTAATTTTGGCATTGCAACCAGCTTTACCCTGCGTA
CCCAGCCGATTGGTGATGTTACCCTGTTTACCATTGCATGGGATTGGGATCGTGGTGCCGAAGCAATTAAAGCATGGCAAGAATGG
CTGGCAACCGCAGATGGTCGCATTAATACACTGTTTATTGCATATCCGCAGGACCAGGATATGTTTGCAGCCCTGGGTTGTTTTGA
AGGTGATGCAGCAGAACTGGAACCGCTGATTGCACCGCTGGTTCATGCAGTTGAACCGACCGAAAAAGTTGCAGAAACCATGCCGT
GGATTGAAGCACTGAGCTTTGTTGAAACAATGCAGGGTGAAGCCATGAAAGCAACCAGCGTTCGTGCAAAAGGTAATCTGAGTTTT
GTTACCGAACCGCTGGGTGATCGTGCCGTTGAAGAAATCAAAAAAGCACTGGCACAGGCACCGAGCCATCGTGCCGAAGTTGTTCT
GTATGGTTTAGGTGGCGCAGTTGCAGCAAAAGGTCGTCGTGAAACCGCATTTGTTCATCGTGATGCACCGGTTGCGCTGAATTATC
ATACCGATTGGGATGATGAAGCCGAAGATGATCTGAATTTTGCCTGGATTCAGAATCTGCGTGCAAGCGTTGCAGCACATACCGAA
GGTCGCGGTAGCTATGTTAATACCATTGATCTGACCGTTGAACATTGGCTGTGGGATTATTATGAAGAAAATCTGCCTCGTCTGAT
GGCCGTGAAAAAACGTTATGATCCGGAAGATGTTTTTCGTCATCCGCAGAGCATTCCGGTTAGCCTGACCGAAGCAGAAGCAGCCG
AACTGGGTATTCCGCCTCATATTGCCGAAGAACTGCGTGCCGCACGTCAGCTGCGTTAG
Polynucleotide encoding SEQ ID NO: 6
SEQ ID NO: 12
ATGGGTAGCAGCCATCACCATCATCATCATGGTAGCAGCGGTGCAAGCGAAGAAGGCAAACTGGTTATTTGGATTAATGGCGATAA
AGGCTATAATGGTCTGGCAGAAGTTGGCAAAAAATTCGAAAAAGATACCGGCATTAAAGTGACCGTTGAACATCCGGATAAACTGG
AAGAAAAATTTCCGCAGGTTGCAGCAACCGGTGATGGTCCGGATATTATCTTTTGGGCACATGATCGTTTTGGTGGTTATGCACAG
AGCGGTCTGCTGGCAGAAATTACACCGGATAAAGCATTTCAGGACAAACTGTATCCGTTTACCTGGGATGCAGTTCGCTATAACGG
TAAACTGATTGCATATCCGATTGCAGTTGAAGCACTGAGCCTGATCTATAACAAAGATCTGCTGCCGAATCCGCCTAAAACCTGGG
AAGAAATTCCGGCACTGGATAAAGAACTGAAAGCAAAAGGTAAAAGCGCACTGATGTTTAATCTGCAAGAACCGTATTTTACCTGG
CCTCTGATTGCAGCAGATGGTGGCTATGCATTCAAATATGAAAACGGCAAATACGATATCAAGGATGTTGGTGTTGATAATGCGGG
TGCAAAAGCCGGTCTGACCTTTCTGGTTGATCTGATCAAAAACAAACACATGAATGCCGATACCGATTATAGCATTGCAGAAGCAG
CATTTAACAAAGGTGAWACCGCAATGACAATTAATGGTCCGTGGGCATGGTCAWATATTGATACCAGCAAAGTGAATTATGGTGTT
ACCGTTCTGCCGACATTTAAAGGTCAGCCGAGCAAACCGTTTGTTGGTGTGCTGAGCGCAGGTATTAATGCAGCAAGCCCGAACAA
AGAACTGGCAAAAGAATTTCTGGAAAACTATCTGCTGACCGATGAAGGTCTGGAAGCAGTGAATAAAGATAAACCGCTGGGTGCAG
TTGCACTGAAAAGCTATGAAGAGGAATTAGCAAAAGATCCGCGTATTGCAGCCACCATGGAAAATGCACAGAAAGGCGAAATTATG
CCGAATATTCCGCAGATGAGCGCATTTTGGTATGCCGTTCGTACCGCAGTGATTAATGCCGCATCAGGTCGTCAGACCGTTGATGA
AGCCCTGAAAGATGCCCAGACCAATAGCATTACCAGCCTGTATGGTAGCAGCGGTAGCTCAGGTAGCAATCTGTATTTTCAGAGCA
ATGGCATGACCGCAGATCCGAGCAGCGAACGTAGCGATATGAATGAAGCAGATGAAGTGAACGAAGTTGATGAACTGAGCGAAACC
GGTCAGACCAGCGGCACCAAAGGTAAACGTCCGTTTACAGGTCGTGTTATTGGTCCGGCAGATGGTGAATTTGATGAAGCACGTCG
TGTTTGGAATGAATGTTTTGCAGCACGTCCGAAAGAAATTGTTTATTGTGCAGATACCCGTGATGTTGTTCGTGCACTGCGTGAAG
TTCGTCAGCGTGGTGGTCCGTTTCGTGTTCGTAGCGGTGGTCATAGCATGAGCGGTCTGAGCGTTCTGGATGATGGTACAGTGCTG
GATGTTAGTGGCCTGGATGATATTCAGGTTAGCGAAGATGCAAGCACCGTTACCGTTGGTAGCGGTGCACATCTGGGTGATATTTT
TCGTGCCCTGTGGGCACGTGGTGTTACCGTTCCGGCAGGTTTTTGTCCGGAAATTGGTATTGCAGGTCATGTTTTAGGTGGTGGTG
CAGGTATTCTGGTGCGTAGCCGTGGTTTTCTGAGCGATCATCTGGTTGCACTGGAAATGGTTGATAGCGAAGGTCGTATTGTTGTT
GCAGATCATGATAGTCATCATGAACTGCTGTGGGCAAGCCGTGGTGGTGGCGGTGGTAATTTTGGCATTGCAACCAGCTTTACCCT
GCGTACCCAGCCGATTGGTGATGTTACCCTGTTTACCATTGCATGGGATTGGGATCGTGGTGCCGAAGCAATTAAAGCATGGCAAG
AATGGCTGGCAACCGCAGATGGTCGCATTAATACACTGTTTATTGCATATCCGCAGGACCAGGATATGTTTGCAGCCCTGGGTTGT
TTTGAAGGTGATGCAGCAGAACTGGAACCGCTGATTGCACCGCTGGTTCATGCAGTTGAACCGACCGAAAAAGTTGCAGAAACCAT
GCCGTGGATTGAAGCACTGAGCTTTGTTGAAACAATGCAGGGTGAAGCCATGAAAGCAACCAGCGTTCGTGCAAAAGGTAATCTGA
GTTTTGTTACCGAACCGCTGGGTGATCGTGCCGTTGAAGAAATCAAAAAAGCACTGGCACAGGCACCGAGCCATCGTGCCGAAGTT
GTTCTGTATGGTTTAGGTGGCGCAGTTGCAGCAAAAGGTCGTCGTGAAACCGCATTTGTTCATCGTGATGCACCGGTTGCGCTGAA
TTATCATACCGATTGGGATGATGAAGCCGAAGATGATCTGAATTTTGCCTGGATTCAGAATCTGCGTGCAAGCGTTGCAGCACATA
CCGAAGGTCGCGGTAGCTATGTTAATACCATTGATCTGACCGTTGAACATTGGCTGTGGGATTATTATGAAGAAAATCTGCCTCGT
CTGATGGCCGTGAAAAAACGTTATGATCCGGAAGATGTTTTTCGTCATCCGCAGAGCATTCCGGTTAGCCTGACCGAAGCAGAAGC
AGCCGAACTGGGTATTCCGCCTCATATTGCCGAAGAACTGCGTGCCGCACGTCAGCTGCGTTAG
Polynucleotide encoding SEQ ID NO: 7
SEQ ID NO: 13
ATGCAGGCATGTAATAATCATCAGCTGACCGATGCAATCCTGCAAGAATTTTCACAGACCCTGAGCGGTGATCTGGTTCTGCCGAC
CGATGGTCTGTATCAGTGGGCACGTCTGATTCATCATACCAATTTTGATGGTATTTATCCGGTGGGCATTGTGTTTTGTGAAACAC
CGGAAGATGTTAGCAAAGCAATTCTGTTTGCACGTCAGTTTGGTCTGCATGTTACCGCACGTGGTGGTGGTCATAGTTATGAAGGT
TATAGCGTTACCGGTGGTCTGCTGATTGATGTTAGCCGTATGAATAGCGTTAGCGTTAATCCGGCAGCAATGACCGCAGTTGTTGG
TCCCCCTCCACTTCTCATCCATCTTTATCATCCCCTCTATTATCCCCACAAACTGAGCATTCCACCTGCTACCTCTCCCAGCCTTC
GTATTGCAGGTTATCTGCTTGGTGGTGGCGTGGGTCTGCAGAGCCGTACCTATGGTGTTGGTTGTGATCGTGTTCTGGAAATTGGT
GTTGTTCTGGCAAGCGGTGAATATGTTGTTGCAAGCCCGACCAATCATAGCGATCTGTATTGGGCATATCGTGGTGGCGGTGGTGG
TAATTTTGGTGTGGTTACCCACTTTAAAATGCAGTGTCATCCGGTTGATCGTCTGAGCTATGCAATTATTACCTGGGATTGGGAAG
CAGCAGCACCGGCATTTAATGCATGGCAGAATTGGCTGAAAGGTCTGGGTGAAGATTATCGCAATTTCTCGATCTTTAAGTTCCTG
GTTAATGCAGGCGGTGATGGTGGTCTGGGTCCGAAAGTTAATCTGATTGTTCAGTTTGATAGCCAGAGCAATACCGGCACCGGTGA
ACTGGAAACCCTGATGGCACCGCTGCTGAATACCGCACATGAACATATTGTTAGCCAGACCATTATGAACGGCGATTTTTTTGAAG
TGACCATGGAAATTATGGCAGGTTGTGCATGGCCGAAAACCGATCTGGATACCGAATTTCTGCGTTGTCATACCGTTGGCAATCCG
GCATTTCCGATGGCAAGCCTGCCTCGTGATACCTATAAAGCAAAAAGCACCTTTTTTGCCGATGTGATTAGCGAAAAAGGTATCGA
AACCTGCATCAAAGCCATTGAAGATCGCTTTAATAGCAATCTGCCGGATAATAGCAGCCAGTTTACCTGTGCACTGCAGTTCGATA
GCGAAGGTGGTATTATGGGTGATGTTCCGAAAGATGCAACCGCATTTATGCATCGTGATTGTCTGATGCATTGTCAGTATCTGGCA
TATTGCCCTGCCGAAGCTGATGCATCGTTTGATGATAACCCCGATTATAGCTATTCTCATCTGACCAATGCTAGCATCCACTCGAT
TAGTGATGCCTTTAATGTTCTGTGGCCGTTTGGTAATGGTCATGCATATCAGAACTATATCGATAAAGAACAGCCGAACTGGCTGT
ATGCATATTATGGTGAAAATGTTGAACGTCTGCGTACCGTGAAAGCAAAATATGATCCGGATAACATCTGGAAGTTTGAACAGAGC
ATTCCGCCTGCATAGGGCGGTAGCGGAGGATCTGGTAGTGGCTCTGGAGGATCTGGTAGTCACCATCATCATCACCAT
Polynucleotide encoding SEQ ID NO: 8
SEQ ID NO: 14
ATGGGCAGCAGCCATCATCATCATCATCACAGCAGCGGCCTGGTGCCGCGCGGCAGCZAACACATTGACTCGGTTGCGCCGGGAGA
CATCCGTTACGAGGATTTGCGCCGCGGTGAAAACTTACGTTTCGTTGGTGATCCAGAGGAAATTCACTTGGTCGGTTCGGCAGCGG
AAATTGAACAGGTTCTTAGTCGCGCGGTGCGCAGTGGGAAACGTGTCGCGGTACGCTCTGGCGGGCATTGCTACGAAGATTTTGTT
GCGAACAGCGACGTGCGCGTGGTTATGGATATGTCCCGCTTAAGCGCAGTGGGCTTTGACGAGGAGCGCGGCGCTTTTGCCGTAGA
GGCTGGGGCCACGCTGGGCGCAGTATACAAGACCTTGTTTCGCGTATGGGGAGTGACTCTGCCAGGTGGCGCCTGTCCTGATGTAG
GCGCTGGCGGACATATCCTTGGCGGTGGATATGGTCCTTTGTCACGCATGCATGGGTCGATCGTCGATTATCTTCACGCTGTTGAG
GTGGTCGTCGTCGACGCTTCTGGAGACGCCCGTACTGTAATCGCTACCCGCGAGCCGAGCGACCCTAACCACGATTTATGGTGGGC
GCACACTGGAGGTGGTGGTGGGAACTTCGGGGTAGTCGTACGCTACTGGCTTCGTACAGCGGAGGCCGACGTACCTCCGGAGCCTG
GGCGCCTGTTGCCCCGTCCACCAGCTGAAGTCTTGCTGAATACTACAGTGTGGCCCTGGGAGGGATTAGACGAGGCCGCGTTTGCT
CGTCTGGTGCGTAATCACGGCCGTTGGTTCGAACAAAACTCGGGACCTGATTCGCCTTGGTGTGACCTTTATAGTGTCTTAGCGTT
GACCCGCTCACAGTCCGGTGCGTTAGCTATGACAACTCAGCTTGACGCAACGGGACCTGACGCCGAAAAACGTCTTGAGACATATC
TTCCGGCTCTTAGCGAAGGCCTTGGGCTTCAGCCCCATTCCGATACACCCCCCTTGCCCTGCTTCCATTCCACACGCTGGCCAGGC
ATCGCCGGCGATGGCGACATGACGGGACGTGCGAAGATTAAGGCTGCCTATGCCCGTCGCAGTTTTGACGATCGCCAAATTGGCAC
ATTATACACACGTCTGACGAGCACCGATTATGATAACCCTGCTGGAGTCGTGGCTCTGATTGCTTATGGCGGAAAAGTCAACGCAG
TACCTGCCGACCGTACTGCCGTAGCGCAGCGCGATTCCATTCTGAAGATTGTATATGTTACGACTTGGGAAGACCCCGCTCAAGAT
CCTGTGCATGTGCGTTGGATCCGCGAGTTATACCGTGACGTTTACGCCGACACGGGGGGTGTGCCTGTTCCTGGGGGTGCGGCCGA
TGGAGCTTACGTTAACTACCCCGACGTGGATTTGGCGGACGAGGAATGGAATACTTCGGGGGTCCCGTGGAGCGAGTTATATTACA
AGGATGCCTATCCTCGTCTGCAAGCTGTGAAAGCACGCTGGGACCCGCGCAACGTTTTCCGTCACGCTTTGTCTGTCCGTGTCCCG
CCAGCTTAG
Clz9 with N-terminal histidine tag and thrombin cleavage site
SEQ ID NO: 15
MKHHHHHHHHGGLVPRGSHGTADPSSERSDMNEADEVNEVDELSETGQTSGTKGKRPFTGRVIGPADGEFDEARRVWNECFAARPK
EIVYCADTRDVVRALREVRQRGGPFRVRSGGHSMSGLSVLDDGTVLDVSGLDDIQVSEDASTVTVGSGAHLGDIFRALWARGVTVP
AGFCPEIGIAGHVLGGGAGILVRSRGFLSDHLVALEMVDSEGRIVVADHDSHHELLWASRGGGGGNFGIATSFTLRTQPIGDVTLF
TIAWDWDRGAEAIKAWQEWLATADGRINTLFIAYPQDQDMFAALGCFEGDAAELEPLIAPLVHAVEPTEKVAETMPWIEALSFVET
MQGEAMKATSVRAKGNLSFVTEPLGDRAVEEIKKALAQAPSHRAEVVLYGLGGAVAAKGRRETAFVHRDAPVALNYHTDWDDEAED
DLNFAWIQNLRASVAAHTEGRGSYVNTIDLTVEHWLWDYYEENLPRLMAVKKRYDPEDVFRHPQSIPVSLTEAEAAELGIPPHIAE
ELRAARQLR
Clz9 with 14-amino acid N-terminal truncation
SEQ ID NO: 16
ADEVNEVDELSETGQTSGTKGKRPFTGRVIGPADGEFDEARRVWNECFAARPKEIVYCADTRDVVRALREVRQRGGPFRVRSGGHS
MSGLSVLDDGTVLDVSGLDDIQVSEDASTVTVGSGAHLGDIFRALWARGVTVPAGFCPEIGIAGHVLGGGAGILVRSRGFLSDHLV
ALEMVDSEGRIVVADHDSHHELLWASRGGGGGNFGIATSFTLRTQPIGDVTLFTIAWDWDRGAEAIKAWQEWLATADGRINTLFIA
YPQDQDMFAALGCFEGDAAELEPLIAPLVHAVEPTEKVAETMPWIEALSFVETMQGEAMKATSVRAKGNLSFVTEPLGDRAVEEIK
KALAQAPSHRAEVVLYGLGGAVAAKGRRETAFVHRDAPVALNYHTDWDDEAEDDLNFAWIQNLRASVAAHTEGRGSYVNTIDLTVE
HWLWDYYEENLPRLMAVKKRYDPEDVFRHPQSIPVSLTEAEAAELGIPPHIAEELRAARQLR
Clz9 with 29-amino acid N-terminal truncation
SEQ ID NO: 17
TSGTKGKRPFTGRVIGPADGEFDEARRVWNECFAARPKEIVYCADTRDVVRALREVRQRGGPFRVRSGGHSMSGLSVLDDGTVLDV
SGLDDIQVSEDASTVTVGSGAHLGDIFRALWARGVTVPAGFCPEIGIAGHVLGGGAGILVRSRGFLSDHLVALEMVDSEGRIVVAD
HDSHHELLWASRGGGGGNFGIATSFTLRTQPIGDVTLFTIAWDWDRGAEAIKAWQEWLATADGRINTLFIAYPQDQDMFAALGCFE
GDAAELEPLIAPLVHAVEPTEKVAETMPWIEALSFVETMQGEAMKATSVRAKGNLSFVTEPLGDRAVEEIKKALAQAPSHRAEVVL
YGLGGAVAAKGRRETAFVHRDAPVALNYHTDWDDEAEDDLNFAWIQNIRASVAAHTEGRGSYVNTIDLTVEHWLWDYYEENLPRLM
AVKKRYDPEDVFRHPQSIPVSLTEAEAAELGIPPHIAEELRAARQLR
Accession ID No. WP_180931280.1 from Streptomyces sp. AJS327 (“Tcz9”)
SEQ ID NO: 18
MATPSAFSGSVLTPGDDGFEAAQVTWNACYSSRPREVMVCHDAASVAEAVRSVRERGLPFRVRSGGHSMCGLSNLDDGVIIDLGGL
GGVELTPDRQTVRIGGGARLADVYNTLWDHRLTVPAGTCPRIGVGGHVLGGGMGVLSRSRGALVDHLTALEMVDAEGRILRVSEDE
NPDLFWACRGGGGGNFGIVTAYELRPTPIDDVTIFTVSWTWSQLPDAVRAWQRWLGSAESRINSFLSLFPQQQDMVVAFGVFDGPA
ADFRPLLAPLTAEVAPEAEVVEEVPFIQAVDTVEALQGEAAAAEQVRAQGSSAIIANPLNDEALATLQEFLTDPPSHRAEVAVYGM
GGVIGERERGDTAFVHRTGLMAFEYRTDWDSPEDDRLNLDWVTRLRHAMAEHTTGAAYVNTIDLALENWLWAYYEENLPRLMAVKR
RYDPENVFHHPHSIPGSLTAEAARAHGVPEATLKRLHDDGLLDGPLD
Accession ID No. WP_173153368.1 from Phytohabitans suffuscus (“Psx9”)
SEQ ID NO: 19
MSTAPVFGGRIVTRLDPSYEEARQVWNAVPDHRPLEIRYCRDAQDVAAALRDALARGLPFAARSGGHSAAALSVVPDGVVIDVSPL
NSISLDQDASQVTFGSGATLGAIYRTLWESGVTVPAGTCPNIGVAGHVLGGGLGILSRSRGPLVDHLIGVTMVDARGELVTADEEN
HSDLLWACRGGGGGNYGIATSFTLRTQPIADVTLFNITWRFDDLGPAVKAWQSWLADADHRINPFMMLYPREQDMVATVGVMEGTP
DAFEPLVKPLLDAVEPSEVVIEPMSFIQAVDTVEALQGEAAVAKSVRALGSSAIIDRPLDDRALETLRRHLIDPPSHRSEVAIYGM
GGKIAAKGREETAFVHRTELMAFEYRTDWDEPADDEKNVAWVRSVRQEMAEHTTGGTYTNTVDLGVENWLWGYYEENLPRLMGVKH
RYDPDGVFQHAHSVPLSLTEAEASRFGIPEQIRRTLRAAGRLT
Accession ID No. WP_030877494.1 from Streptomyces varsoviensis (“Svx9”)
SEQ ID NO: 20
MATSARFAGTVLAPGDPGFEDASRTWNACFASRPREVMVCHSTAEVADAVREVRERGVSFRVRSGGHSMAGLSSVEDGVVIDLGGL
DAISLSADRSLVTVGGGTRLGDIYRELWRSGVTVPAGTCPRIGIGGHVLGGGMGVLSRSRGALVDHLAALEFVDAEGRAYRVDENE
HPDLFWACRGGGGGSYGIATSYTLRTSPVDEVTLFTLSWNWERFAPAVRAWQSWLASTDDRVNTFLSLFPRQQDLVAAFGVYDGPA
AEFEELLRPLVAAVRPQDQVVEPMPFIQAVDTVELLQGEAAAADQVRAQGSSAIIGGPLDGAAIATLQQFLTDPPSHRSEVAVYGM
GGAIARPARTDTAFVHRTGLMAFEYRTDWDRSEDDAKNLAWIKALRHAMAPHTTGAAYVNTIDLALENWLWAYYEENLPRLIEVKR
RYDPGDVFHHPHSIPLTVTEADIARYGIPEAVAARLRAAGLTDRQ
Accession ID No. WP_235454663.1 from Streptomyces olivochromogenes (“Sox9”)
SEQ ID NO: 21
MGTPARFTGTVLTPGDAGFEDASRTWNACYASRPREVMVCHSAEAVAEALGSARGRGIPFRVRSGGHSMAGLSSVADGAIIDLGGL
DAITLSEDSSLVTVGGGARLGDIYRELWKAGVTVPAGTCPRIGIGGHVLGGGMGVLSRSRGALVDHLVGLELVGADGRLRQVDDDE
HADLFWACRGGGGGSFGIATSYTFRTSPISEVTIFTIAWSWAQFAPAVKAWQGWLASAEDRINTFLSLFPRQQDLLVAFGVFDGPA
AEFEELLRPLVAAAEPQTLVVEPVPFIQAVDTVEALQGEAAAADQVRAQGSSAIVAEPLNEAAIATLQEFLTDPPSHRAEVAVYGM
GGAIGHRARTDTAFVHRTGLMAFEYRTDWDLPEDDGKNLAWVRALRHSMAEHTTGAAYVNTIDLALENWLWAYYEENLPRLMQVKR
QYDPGDVFQHPHSIPVSLTAEDIARHGIPEQVAAEIRSAGLLS
Accession ID No. ANW12117.1 from Streptomyces albogriseolus (“ThdO”)
SEQ ID NO: 22
MDRYPRNAGTVVSPADPDYERARRIWNNRFDCRPRAIMYPRDEAEAAAALGYARDNEVPFRIRSGGHNSEGFCTVDDGIIIDLSKL
GSIAVNNDRTRAVISPGVMLGEVYANLWSVGGTIPAGVCPDIRIGGHVLGGGIGMLVRSRGLLIDNLVGLTVVDARGERLQVDADH
HPDLFWACKGGGGGNYGIATSYTFEMRPISDVTIFKLQWDWQGGMAVLDAWQHWLLSADSRVNARFNVFSSTVGTVMTVGLFEGSA
DELKSILQPLVEKFPPRDSFVRTMPYMECVGTFSEQVPSIRAKFVPALSAGPLDADALKTLERWHKDAPTGVKTGLYGLGGEVLGS
VSPDSSAFAHRDASICVEYLGHWQGAEHDREHLGWLSGVREEMDAFMTGGAYVNSPDRDLENWLHAYYGESLPRLMDVKRRYDPDN
VFAFEQSIPGSLTLPEARAAGLTDTVVEDLRARGLLSA
Accession ID No. WP_067055472.1 from Streptomyces sp. FXJ1.172 (“ThnG”)
SEQ ID NO: 23
MGAYPDTIGTVVTPGDTRYERARRLWNERFDYRPRAVVLPGDSHETAAALRYVRERGIPFRIRSGGHNSEGFSGLDDGIVIDVSRL
GALAVYGDRTRATLGPGVLLGEAYAALWALGGTIPAGVCPDIRIGGHVLGGGIGMLVRSRGLLIDHLVALTLIDARGEELTVDADH
HPDLLWASRGGGGGNFGIVTSYTFAMRPIGDVTLFTLEWDARDGIAALDAWQHWLSQADPRVNSRFNLFPASADRALATGLFEGGE
RELTALLRPLTNACRPRATSVRALPYGDSIGTWTEQVPSVRAKFTPALAEGPLGAEALRTLGRRHEKAPPGVKTGLYGLGGAVLAD
VPPDHSAFAHRAAFLCVEYLGHWSDPGSDAEHLDWLSGTRAEMLPFMTGGAYVNSPDLDLENWLHAYYGDGLPRLMEVKRRYDPHG
LFTFEQSIPTALTRAQARAARLPGPVVADLHARGLLTD
Accession ID No. WP_098755161.1 from Streptomyces sp. Ru87 (“SrThX”)
SEQ ID NO: 24
MEEHPQHVGTVVTQADPDYEQARHIWNERFDFRPRAVFYPRNAEETAAAVTHVRDRGLPFRIRSGGHNSEGFCSVDDGVVIDLSKL
ASIGVNNDRTRATTGPGVLLGEVYATLAAVGGTIPAGVCPDIRIGGHVLGGGIGMLVRSRGLLIDSLVGLTLVDARGRILEVDDSS
HPELMWACRGGGGGNYGIVTSYTFEMRPVSEVTLFTLEWDWNGGIGVIDTWQHWILRADTRVNARFAFLPGSTGTVMSTGLFEGPA
RELESILRPLVGKHPPRDSVVRTVPYAESVGTFTEPVPSIRAKFVPALAGGPLGTEALEILERRHRDAPAGVKTGLYGLGGAVLDN
APAKRSSFAHRDAPICVEYLGHWKDSAHDSEHLDWLTATREELHGFMTGGAYVNSPDRDLADWLHAYYGENLPGLMDVKRRYDPDD
VFSFEQSIPTSLSPAEARAAGLTDTVVAELRGRGLLRS
Accession ID No. A0A1H4CL41
SEQ ID NO: 25
MSQTGRTISPSDPEYDEARKGWNDVYQSHPTEIIFCCNADEVASALKRCTEQQLPFRVRSGGHSLEGYSALNDGAVIDLSGMNSLV
LNDDTTEVTLGPGARLGDVYSDLFANGVTVPGGTCPRVAVGGLVLGGGYGYLSRTYGMLAQRALSFEIVAADGRILTASKDEHADL
FWACRGGGGGIFGIMTAVTLKTVPISDVATCRLVWSWDHFAEVFDAYQRWLPTSDSRLNTLLFLPAQHYGKIALSVLCTTTTTEEL
EPLLQSLLDVVPAPRETKYAVIPYIERVGNEDGLIEPQVYTRTRFGALQPYFRQPLDAAGIEALRRMHAEATGNTITIVCGAGSQV
FTKMAENDPVSFPHLDALMSFDIRIDWSDPDLDHVYFDWLSAIDQELALYRSGTYLNYNDFNIDDALYPRYLDSFARLVAIKHRYD
PDNTFQFPASIPPSLTEDEAERMQLPDKVIKELREYGRLT
Accession ID No. WP_211768552.1 (“Kcx9”)
SEQ ID NO: 26
MSRSQRVAATVTAADPGYDQARRIWNERFDCRPLAVLYPADVAEVAGAVRAARDDGVPFRIRSGGHNVEGFSVLDDGYVIDLSRLT
GVTVDDDRTRGTVAAGTLLGDVYSGLWAAGVTIPAGVCPDIRIGGHVLGGGVGMLVRSRGMLVDSLVGLTMVDADGEVLEVNDDTH
PDLMWACRGGGGGNFGVATSYTFHTRPIGDVTLVTAAWNFDNAITALDRWQHWAVEVDQRVNARFSLFSPGTGGGRMAVLFEGTSA
EWESLARPLFEQCPPAEHTVRETAYINSVGQFSQRVPSIRAKFVPALAAGPLDAEALKILERHHRAAPDGVKTSFYGLGGAVLGAD
LAKRSAFAHRDALFCVEYLGHWHGAEHDAEQLGWLAGIRDELRPFMTGGAYVNSPDRDLTDWLHAYYGASLPRLMEVKRRYDPEDV
FRFEQSIPVSLSPAAARAAGLTEAVVADLGARGLLTS
Accession ID No. A0A7X0U8H0
SEQ ID NO: 27
MAHRKSPARIAKAARVAQFHALAASAADAAGGAGETSDTAIALESLAHLEHMLVGRVVLPSSPDYDQARQESNPAFQSFPKIIVYC
AVENDVLECLAVARKYKVPVAVRCGGHSTAGYSVNNGMVIDLSDIQGVVLDPPNQRVHVRPGTDFDHFNGAMNHTGWHVPTGACGS
VCVGGFVQGGGYGYTSRRYGIQSDLVESLRVALADGTIVTASETENPLLYWSMRGGTGGNFGVLLQVTYTMVPLDTVWAWSIGWDM
SQAANVLTMMQQRYMQSGAPDALGYMMNLGYYGGQPVAMVQGMFCGTREAGLQAIAPLMEIPSAQLLVDKTGTYPDMNIWLEDHPY
SLPDNLPDGIAESKASGYISRPLTTADWQSIIDYFKTSPNQWSLAYLEPYGGAINRYPIEASAFIHRRVDADLVVDVFWRNPAERA
QTEAWLAGFMQLVQPFLNGHVYQNYPDASLQDFENAYWGPAYPTLQQAKRAYDPTNFFKFQQSIRLPREE
Accession ID No. A0A1Q5S5E2
SEQ ID NO: 28
MKSLGRREREKALRVQGWAGGISLDPRFTKDDLAALTMKVQGDIVLPTDAGYQQDRQLANNAFQSFPQLIVYCEVFSDIRQCLAFA
QRCGLTVAVRSGGHSTAGLSVNDDMVIDLSRLRYVIVDPKAHTAVVGAGTNMGHLNAALDIYHLHVPGGSCHDVCVAGFMQGGGFG
YTSRCFGLNCDNVVAIRVMLADGRIVVADDKVNRDLFWAVRGGTGGNFGILLEITYRLHEVKSVWAFRLQWPLRAGSDDTVRAARL
LSIIQSEFTRKAPKELGYMTFLGWQGDMPYLIIRGMYVGPAERGKALLQPLLDVPGAEWINDRSGSYAEMMHYLVSTTPDLPQVPD
LAREDKQSGYVARPLGPDDWQEVIEQFLETPNNSSMLAMEHYGGAISERHEHDMAFVHRRVDMDVYLDVFWMNEPERVDAVTFLDR
FMRFMERFCNGQSYQNYPRLAQTDYRRRYWGDVFDDLLAVKRKYDPGTFFRSAQGISADPSLSATDPVSTLTGLLDGIESEADPV
Accession ID No. A0A0Q7FI10
SEQ ID NO: 29
MVSGRRRFLQVGAAGAAGAAALALAPSAVLAKPALKLGPHNLRALARDFDGRLILPGQEGYALAAWPNNARYAGVLPQAVAMCANQ
KDVQRCIRWAGDSGMPFAIRSGGHNYAGFSTTPGLLIDVKAMNSVTVELKNETAWVQGGANNNDVAAVLRNYPFAIPSGRCPTVGT
SGLVLGGGWGFAATHDGLTCDSLLGTELVLASSEARSINANSHKELFWAVRGGGGGNFGVHTGFTFQLHKVGKVTTFNLSWAPGKQ
IELLRALQALQLAYPTMISTRSKALPSAAGPRPARDTLYVQTLGQFWGGEKELREMLAPLYAIAVPQTADIYEVDYWRGRDYLLTD
DPNGLYDIRCSFVHKELSEAALDNMLNWMTQWPGGSVLQENMGILFAIGGQVKAVRSDATAYVHRDSNFIFEMEASWGPIDTPETV
QRQRAWLKDYYNDMQRYLQPESYVNFPNRDLKNWAQAYYGKNLEQLSAVKRHVDPGNLFRFEQSIPLAPKR
Accession ID No. A0A2E0XWX6
SEQ ID NO: 30
MMNPTSRRTFLRATGATAIMVGATPLAAWKQACNNHQLTDAILQEFSQTLSGDLVLPTDGLYQWARLIHHTNFDGIYPVGIVFCET
PEDVSKAILFARQFGLHVTARGGGHSYEGYSVTGGLLIDVSRMNSVSVNPAAMTAVVGAGAVLIDVYHGLYYPHKLSIPGGSCPSV
GIAGYLLGGGVGLQSRTYGVGCDRVLEIGVVLASGEYVVASPTNHSDLYWAYRGGGGGNFGVVTHFKMQCHPVDRLSYAIITWDWE
AAAPAFNAWQNWLKGLGEDYRNFSIFKFLVNAGGDGGLGPKVNLIVQFDSQSNTGTGELETLMAPLLNTAHEHIVSQTIMNGDFFE
VTMEIMAGCAWPKTDLDTEFLRCHTVGNPAFPMASLPRDTYKAKSTFFADVISEKGIETCIKAIEDRFNSNLPDNSSQFTCALQFD
SEGGIMGDVPKDATAFMHRDCLMHCQYLAYWPAEGDAWFDDNPDYSYCDVSNGSMQWISDAFNVLWPFGNGHAYQNYIDKEQPNWL
YAYYGENVERLRTVKAKYDPDNIWKFEQSIPPA
Accession ID No. D9XHS6
SEQ ID NO: 31
MVTMARNRLFHAPTVGTEGRAAHAASSAAAPPRAPGHRGAKVDWDRLRQRMRGALLRPGDPGFEDARKVFNPLNDDHVPVAVAQCE
STSDVSEAVRAAASRVPLAARSGGHSYVGYSAPHGGLALDLRRMSAVEVQSDGVATVGAGAPLRDVYGGVAQANRCLPAGSCFTVG
VAGVTLGGGIGVLQRRFGLTCDHLVGAEMVTADGRTLTVSAARTPDLFWALRGGGGGNFGIVTQFTFATDPAPALTVFVVGFPPGK
VPEVLSAWQSWISAAPRELWANCNMTGGDVPSCRVAGCFVGPSSSCNPLLDDLISRSGVLPNRRTVQDREYFSAMRFFSGSPERQS
FVASSRILSEQASSPDAVAGLLVGRPGVSIILDPLGGAVADVGVQQTAFPHRKAFATAQIYASATAASEAEVTRTVRDVVAGLASL
GLGGGYVNYIDPALPDWAQAYYGPNLQRLRSVAHDYDPDGVFDFPQGLTRT
Accession ID No. A0A0K3BN04
SEQ ID NO: 32
MINRRTLLRTGLGTAGALLLAPATAAASTRRTRWERLSGCLSCDVVLPSDPAYTQARQLDSAAFDHVLPQAIAYCENVGDVRTCVR
FAQDNDIAVAVRSGGHSAAGYSTTTGMVLDVSRLSRIDVGASSVRLGPGVQGIDLLSTLSQRGLSAITGNCPTVGMGGYLQGGGVG
PLSRKYGVASDRLLSAEVVLADGRVARCSPTREPDLFWALRGGGGGNFGVVTQYEIAPVTTTRTVSFSLSWPYARAAEVIAAFLPW
LASAPDDLTANLTVMNQNAAPGSQPSLFVSGSWFGGPVATVDPHLTALVSAVGSAPASRSADDLTYFDAMMRMFGCGTKTVQQCHR
VGYNLEATLPRQSFVLARGRLADRPMTSSAIGEALAAYEADPAGGQFRVMSWGGLGGRINRVAPSATAYAHRTAQHYFTYTAGVRT
AAPPDEVKASIESWTAKGFAVARRHGNGEGQVNFIDSTLPDWRRAYYGGNHSRLQWIKWRYDPHRFFRFAQSI
Accession ID No. A0A1U9QQ65
SEQ ID NO: 33
MTSRRSFLVGAGATALAVTAFQGSSAARTASGGKWHQLRRHLRGRLVLPSDPDYRRAKQLYLIQFDDSRPRAIAYCASAADVALCL
RFAQDHALPIAVRSGGHSAGGYSTTEGLVIDVSRIDSTVLGRDTATIGPGAQLVDITNTLAPAGLTISGGYCPTVAAGGYFAGGGM
GLFTRSIGMASDKVTSAEVVLADGSVVTASPRRNSDLYWAVRGGGGGNFGVVTSYEVTPTPLTDLAACNLSWRFDQALDMLDGWAR
WLVDAPRAIGSGLLVTLEDAAPGKVPMATIFLGSVDTGGGFDGEIDRLVSLIGHAPSFRQTHTAPYQDILMDLYQCGGLTVQQCHR
ADTSPGGQLTRPAFGLWRSRLFGEDAMPREGWAKALSVLDSERQAGQMRQLQVVAIGGAVNSVSRGATAYVHRDSLFSASFLTSNS
VAPVPDEAKESAGRFVDAGFAVLDPYSNGETYQNFYDSRLPDWQSSYYAENYPRLKRVKRSYDPHNIFRFAQSIR
Clz9-var1
SEQ ID NO: 34
MTADPSSERS DMNEADEVNE VDELSETGQT SGTKGKRPFT GRVIGPADGE FDEARRVWNE
CFAARPKEIV YCADTRDVVR ALREVRQRGG PFRVRSGGHS MSGLSVLDDG TVLDVSGLDD
IQVSEDASTV TVGSGAHLGD IFRALWARGV TVPAGFCPEI GIAGHVLGGG AGILVRSRGF
LSDHLVALEM VDSEGRIVVA DHDSHHELLW ASRGGGGGNF GIATSFTLRT QPIGDVTLFT
IAWDWDRGAE AIKAWQEWLA TADGRINTLF IAYPRDQDMF AALGLFEGDA AELEPLIAPL
VHAVEPTEKV AETMPWIEAL SFYETMQGEA MKATSVRAKG NLSFVTEPLG DRAVEEIKKA
LAQAPSHRAQ VILYGLGGAV AAKGRRETAF VHRDAPVALW YHTAWDDEAE DDLNFAWIQN
LRASVAAHTE GRGSYVNFID LTVEHWLWDY YEENLPRLMA VKKRYDPEDV FRHPQSIPVS
LTEAEAAELG IPPHIAEELR AARQLR
Clz9-var2
SEQ ID NO: 35
MTADPSSERSDMNEADEVNEVDELSETGQTSGTKGKRPFTGRVIGPADGEFDEARRVWNECFAARPKEIVYCADTRDVVRALREVR
QRGGPFRVRSGGHSMSGLSVLDDGTVLDVSGLDDIQVSEDASTVTVGSGAHLGDIFRALWARGVTVPAGFCPEIGIAGHVLGGGAG
ILVRSRGFLSDHLVALEMVDSEGRIVVADHDSHHELLWASRGGGGGNFGIATSFTLRTQPIGDVTLFTIAWDWDRGAEAIKAWQEW
LATADGRINTMFHAYPRDQDMFAALGLFEGDAAELEPLIAPLVHAVEPTEKVAETMPWIEALSFYETMQGEAMKATSVRAKGNLSF
VTEPLGDRAVEEIKKALAQAPSHRAQVILYGLGGAVAAKGRRETAFVHRDAPVALWYHTAWDDEAEDDLNFAWIQNLRASVAAHTE
GRGSYVNFIDLTVEHWIWDYYEENLPRLMAVKKRYDPEDVFRHPQSIPVSLTEAEAAELGIPPHIAEELRAARQLR
Clz9-var3
SEQ ID NO: 36
MTADPSSERSDMNEADEVNEVDELSETGQTSGTKGKRPFTGRVIGPADGEFDEARRVWNECFAARPKEIVYCADTRDVVRALREVR
QRGGPFRVRSGGHSMSGLSVLDDGTVLDVSGLDDIQVSEDASTVTVGSGAHLGDIFRALWARGVTVPAGFCPEIGIAGHVLGGGAG
ILVRSRGFLSDHLVALEMVDSEGRIVVADHDSHHELLWASRGGGGGNFGIATSFTLRTQPIGDVTLFTIAWDWDRGAEAIKAWQEW
LATADGRINTMFHCYPRDQDMFAALGLFEGDAAELEPLIAPLVHAVEPTEKVAETMPWIEALSFYETMQGEAMKATSVRNKGNLSF
VTEPLGDRAVEEIKKALAQAPSHRAQVILYGLGGAVAAKGRRETAFVHRDAPVALWYHTAWDDEAEDDLNFAWIQNLRASVAAHTE
GRGSYVNFIDLTVEHWLWDYYEENLPRLMAVKKRYDPEDVFRHPQSIPVSLTEAEAAELGIPPHIAEELRAARQLR
Clz9-var4
SEQ ID NO: 37
MTADPSSERSDMNEADEVNEVDELSETGQTSGTKGKRPFTGRVIGPADGEFDEARRVWNECFAARPKEIVYCADTRDVVRALREVR
QRGGPFRVRSGGHSMSGLSVLDDGTVLDVSGLDDIQVSEDASTVTVGSGAHLGDIFRALWARGVTVPAGFCPAIGIAGHVLGGGAG
ILVRSRGFLSDHLVALEMVDSEGRIVVADHDSHHELLWASRGGGGGNFGIATSFTLRTQPIGDVTLFTIAWDWDRGAEAIKAWQEW
LATADGRINTMFHCYPRDQDMFAALGLFEGDAAELEPLIAPLVHAVEPTEKVAETMPWIEALSFYETMQGEAMKATSVRNKGNLSF
VTEPLGDRAVEEIKKALAQAPSHRAQVILYGLGGAVAAKGRRETAFVHRDAPVALWYHTAWDDEAEDDLNFAWIQNLRASVAAHTE
GRGSYVNFIDLDVEHWLWDYYEENLPRLMAVKKRYDPEDVFRHPQSIPVSLTEAEAAELGIPPHIAEELRAARQLR

### Examples

### Example 1: Clz9-var4 immobilization assay —Process and Figures

Cloning. The synthetic gene coding for Clz9-var4 contains the following mutations to WT-Clz9 (Uniprot: U6Δ1G7): D404A, T438F, V323Y, N400 W, Q275R, C₂₈₅L, E370Q, V372I, L269M, I271H, Δ338N, Δ272C, E159A, T442D. Clz9-var4 was cloned into an expression vector containing a 6x poly-histidine tag at the N-terminus. This vector requires the addition of IPTG and cumate to induce protein expression.

Enzyme Expression. The expression plasmid containing Clz9-var4, pg 21284, was used to transform E. coli BL21 (DE3), and a single colony was used to inoculate a 20-mL preculture of LB media and grown overnight at 35° C. 10 mL of the preculture was used to inoculate 1 L LB medium in 2.8-L Fembach flasks supplemented 100 g/mL of carbenicillin and grown at 37° C. until OD₆₀₀=0.6. Protein expression was induced by the addition of 0.5 mM isopropyl P-thiogalactoside (IPTG) and 0.2 mM cumate and the temperature was lowered to 18° C. for 20 h. Cells were harvested by centrifugation at 8000×g for 10 min. The cell pellet was frozen and stored at −20° C. until purification.

Enzyme Immobilized Activity and Purification. Frozen cells were resuspended in 50 mL of 50 mM potassium phosphate buffer, pH 8.0 with 300 mM KCl and 10 mM imidazole supplemented with a Pierce Protease Inhibitor tablet, EDTA free, benzonase, and lysozyme (Millipore Sigma). Cells were lysed by passage through a Microfluidics microfluidizer at 4° C. Cell debris was removed by centrifugation at 29,000×g for 30 min followed by syringe filtering with a 0.45 m filter. The resulting solution was loaded onto a 5-mL HisTrap HP (GE Healthcare) nickel affinity column that had been pre-equilibrated with resuspension buffer using an ÄTKA HPLC system. After loading Clz9-var4 onto the column, it was washed with reaction buffer 100 mM Tris-HCl buffer, pH 7.4 containing 0.1% TritonX-100 and 2% DMSO. A 200 gM solution of CBGA in 100 mM Tris-HCl buffer, pH 7.4, with 0.1% TritonX-100 and 2% DMSO was passed through the column at three different flowrates: 0.1 mL/min, 1 mL/min and 3 mL/min. Fractions of 2 mLs were collected until 50 mLs of the CBGA solution was passed through the column. The column was re-equilibrated with resuspension buffer, and the protein was eluted using 50 mM potassium phosphate buffer, pH 8.0 with 300 mM KCl and 300 mM imidazole over a gradient of 15 column volumes. Fractions of 3.0 mLs were collected and tested for purity by SDS-polvacrylamide gel electrophoresis. Typically, Clz9-var4 eluted between 6-12 column volumes. Fractions containing the purest samples were pooled. Clz9-var4 was exchanged and concentrated into 20 mM HEPES, pH 7.4 with 10 mM KCl by four repeated concentration and resuspension steps using an Amicon Ultra-15 Centrifugal Filter Unit with 30 kDa cutoff. Final protein concentration was determined by Bradford assay (BioRad, Hercules, CA), absorbance at 280 nm, estimated purity, and calculated molecular weight and extinction coefficient of the enzyme. Final yield was 70 mgs Clz9-var4 with the concentrated enzyme solution appearing dark-gold/brown in color. The collected UV spectra of Clz9-var4 is representative of a bi-covalent FAD attachment to the enzyme and activity measurements were as expected.

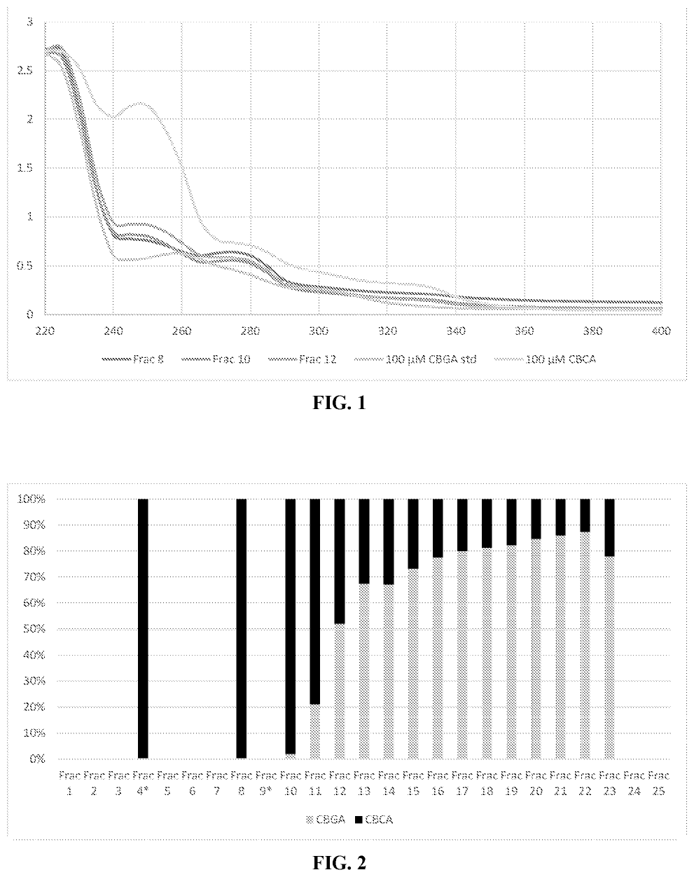

Analytical Method. The 25×2 mL fractions collected from the CBGA passage through the column containing immobilized Clz9-var4 were analyzed for CBCA formation using two methods. The UV-spectra (220 to 400 nm) of a 5-fold diluted sample into Tris buffer, pH 7.4 were collected and compared to CBGA and CBCA standard solutions at similar concentrations (FIG. 1). In addition, the 30 Ls of each fraction was quenched into 270 L of 75% acetonitrile solution containing 0.1% fonnic acid and loaded onto an LC/MS system (FIG. 2). CBCA and CBGA were quantified by relative peak area versus peak area of known concentrations of CBCA and CBGA cannabinoid standards.

What is claimed is:

1. An in vitro method for producing a cannabinoid. the method comprising reacting a prenylated aromatic compound with a flavin-dependent oxidase in a reaction mixture to form the cannabinoid, wherein the flavin-dependent oxidase is not derived from C. sativa.

2. The method of claim 1, wherein the flavin-dependent oxidase is less than 85% identical to a flavin-dependent oxidase from C. sativa.

3. The method of claim 1, wherein the flavin-dependent oxidase does not comprise a disulfide bond.

4. The method of claim 1, wherein the flavin-dependent oxidase is immobilized on a solid support.

5. The method of claim 1, wherein the flavin-dependent oxidase is prokaryotic or fungal.

6. An in vitro method for producing a cannabinoid. the method comprising reacting a prenylated aromatic compound with a flavin-dependent oxidase in a reaction mixture to form the cannabinoid, and wherein the flavin-dependent oxidase does not comprise a disulfide bond.

7. The method of claim 6, wherein the flavin-dependent oxidase is immobilized on a solid support.

8. The method of any one of claims 1 to 7, wherein the flavin-dependent oxidase is a berberine bridge enzyme (BBE)-likc enzyme

9. The method of any of claims 1 to 8, wherein the flavin-dependent oxidase is a prokaryotic or fungal protein or derivative thereof.

10. The method of claim 1 to 9, wherein the flavin-dependent oxidase comprising:
(i) a first amino acid sequence comprising a His residue, wherein an FAD cofactor is covalently attached to the His residue; and
(ii) a second amino acid sequence comprising a peptide motif of Formula I:
[Formula I]
X₁-Gly-X₂-Cys-X₃-X₄-X₅-X₆-X₇-X₈-Gly-X₉-X₁₀-X₁₁-Gly-
Gly-Gly-X₁₂-Gly
wherein each X is any amino acid; and wherein the FAD cofactor is covalently attached to the Cys residue,
wherein the flavin-dependent oxidase is capable of oxidative cyclization of the prenylated aromatic compound into the cannabinoid.

11. The method of any one of claims 1 to 10, wherein the flavin-dependent oxidase comprises at least 70% sequence identity to any of SEQ ID NO:1, SEQ ID NO:3, SEQ ID NO:16, or SEQ ID NO:17, or any one of SEQ ID NOs:34-37.

12. The method of any one of claims 1 to 11, wherein the flavin-dependent oxidase comprises at least 70% sequence identity to any one of SEQ ID NOs:18-33.

13. The method of any one of claims 1 to 12, wherein the flavin-dependent oxidase comprises an affinity tag.

14. The method of claim 13, wherein the affinity tag is selected from an AUl epitope tag, an AU5 epitope tag, a bacteriophage T7 epitope tag, a bacteriophage V5 epitope tag, a bluetongue virus tag, a calmodulin binding peptide tag, a cellulose binding domain tag, a chitin binding domain tag, a E2 epitope tag, a FLAG epitope tag, a Glu-Glu (EE-tag) tag, a Human influenze hemagglutinin tag, a histidine affinity tag, a HSV epitope tag, a KT3 epitope tag, a Myc epitopc tag, a PDZ ligand tag, a polyarginine tag, a polyaspartate tag, a polycysteine tag, an polyhistidine tag, a polyphenylalanine tag, a Protein C tag, an Si tag, an S tag, a streptavadin binding peptide tag, a strep-tag, a TrpE tag, a Universal (HTTPHH) tag, or a VSV-G tag.

15. The method of claim 14, wherein the polyhistidine tag is a 6X histidine tag.

16. The method of claim 15, wherein the flavin-dependent oxidase comprises at least 70% sequence identity to any of SEQ ID NO:4, SEQ ID NO:5, SEQ ID NO:6, or SEQ ID NO:15.

17. The method of any one of claim 1 to 16, wherein the flavin-dependent oxidase is immobilized on a solid support by an interaction with an affinity tag.

18. The method of any one of claim 1 to 16, wherein the flavin-dependent oxidase is immobilized on a solid support by an interaction with an antibody.

19. The method of any one of claim 1 to 16, wherein the flavin-dependent oxidase is immobilized on a solid support by a covalent bond with the solid support.

20. The method of any one of claims 1 to 19, wherein the cannabinoid is CBCA, CBC, CBCOA, CBCVA, THCA, THCA, THCV, THCO, THCVA, THCOA, THC, CBDA, CBDV, CBDO, CBDVA, CBDOA, CBD, CBCA, CBCV, CBCO, CBCVA, CBCOA, CBC, cannabinolic acid (CBNA), cannabinol (CBN), cannabicyclol (CBL), cannabivarin (CBV), cannabielsoin (CBE), cannabicitran, an isomer, analog or derivative thereof, or a combination thereof.

21. The method of claim 20, wherein the cannabinoid is CBCA, CBC, CBCOA, CBCVA, or THCA.

22. The method of any one of claims 1 to 21, wherein the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), cannabigerol (CBG), or combinations thereof.

23. The method of claim 22, wherein the prenylated aromatic compound is canmabigerolic acid (CBGA).

24. The method of any one of claims 1 to 23, wherein the prenylated aromatic compound is cannabigerolic acid (CBGA), and the cannabinoid produced is CBCA, CBDA, or THCA.

25. The method of any one of claims 1 to 24, wherein the reaction mixture has a pH of about 4.0 to about 8.0.

26. The method of any one of claims 1 to 25, wherein the reaction mixture comprises a solvent.

27. The method of claim 26, wherein the solvent is one or more of aqueous buffer, dimethyl sulfoxide (DMSO), dimethyl fonnamide (DMF), iso-propyl alcohol, ethanol, and cyclodextrin.

28. The method of claim 26 or claim 27, wherein the amount of the solvent in the reaction mixture is between 5% and 30% (w/v).

29. The method of any one of claims 1 to 28, wherein the reaction mixture comprises a solubility additive.

30. The method of claim 29, wherein the solubility additive comprises a nonionic surfactant.

31. The method of claim 30, wherein the nonionic surfactant comprises a polysorbate or polyethylene glycol tert-octylphenyl ether.

32. ‘An in vitro method for producing a cannabinoid, the method comprising reacting a prenylated aromatic compound with a flavin-dependent oxidase in a reaction mixture, wherein the flavin-dependent oxidase is a prokaryote protein or fungal protein, or derivative thereof.

33. The method of claim 32, wherein the flavin-dependent oxidase is immobilized on a solid support.

34. An in vitro method for producing a cannabinoid, the method comprising reacting a prenylated aromatic compound with a flavin-dependent oxidase in a reaction mixture, and wherein the flavin-dependent oxidase comprises at least 70% sequence identity to any of SEQ ID NOs:1-6 or any of SEQ ID NOs:15-37.

35. The method of claim 34, wherein the flavin-dependent oxidase is immobilized on a solid support.

36. An in vitro method for producing a cannabinoid, the method comprising:
a. culturing a prokaryote cell or fungal cell comprising a flavin-dependent oxidase in a fermenter;
b. lysing the prokaryote cell or fungal cell to form a lysate comprising the flavin-dependent oxidase;
c. adding a prenylated aromatic compound to the flavin-dependent lysate in vitro, whereby the prenylated aromatic compound can interact with the flavin-dependent oxidase to form the cannabinoid; and
d. recovering the cannabinoid.

37. The method of claim 36, wherein the prenylated aromatic compound are added to the flavin-dependent oxidase after one or more impurities in the lysate have been removed.

38. The method of claim 36 or claim 37, wherein the flavin-dependent oxidase does not comprise a disulfide bond.

39. An in vitro method for producing a cannabinoid. the method comprising:
a. culturing a prokaryote cell or fungal cell comprising a flavin-dependent oxidase in a fermenter, wherein the flavin-dependent oxidase does not comprise a disulfide bond;
b. lysing the prokaryote cell or fungal cell to form a lysate comprising the flavin-dependent oxidase;
c. adding the lysate to a chromatography apparatus comprising a solid support, whereby the flavin-dependent oxidase is immobilized onto the solid support;
d. adding a prenylated aromatic compound to the chromatography apparatus, whereby the prenylated aromatic compound can interact with the flavin-dependent oxidase to form the cannabinoid; and
e. recovering the cannabinoid.

40. A system for producing cannabinoid products, comprising:
a. a fermenter holding a cell culture medium comprising prokaryote cells or fungal cells producing a flavin-dependent oxidase wherein the flavin-dependent oxidase does not comprise a disulfide bond;
b. a means for lysing the prokaryote cells or fungal cells to form a lysate comprising the flavin-dependent oxidase;
c. an apparatus containing a reaction mixture configured to interact the immobilized flavin-dependent oxidase with a prenylated aromatic compound to form a cannabinoid which is produced by the flavin-dependent oxidase.

41. A system for producing cannabinoid products comprising:
a. a fermenter holding a cell culture medium comprising prokaryote cells or fungal cells producing a flavin-dependent oxidase wherein the flavin-dependent oxidase does not comprise a disulfide bond;
b. a means for lysing the prokaryote cells or fungal cells to form a lysate comprising the flavin-dependent oxidase:
c. an apparatus containing a reaction mixture configured to immobilize the flavin-dependent oxidase onto a solid support, and then interact the immobilized flavin-dependent oxidase with a prenylated aromatic compound to form a cannabinoid which is produced by the flavin-dependent oxidase.

42. A biphasic composition comprising:
a. a prenylated aromatic compound in a first phase, wherein the first phase comprises an organic solvent;
b. a flavin-dependent oxidase in a second phase, wherein the second phase comprises an aqueous solvent, and wherein the flavin-dependent oxidase does not comprise a disulfide bond.

43. A biphasic composition comprising:
a. a prenylated aromatic compound in a first phase, wherein the first phase comprises an organic solvent;
b. a flavin-dependent oxidase immobilized on a solid support in a second phase, wherein the second phase comprises an aqueous solvent, and wherein the flavin-dependent oxidase does not comprise a disulfide bond.


SEQUENCE LISTING (37 sequences)

SEQ ID NO: 1 - protein, 462 aa, unidentified
MQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHTGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 2 - protein, 462 aa, synthetic construct
MQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHVGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 3 - protein, 506 aa, Streptomyces sp.
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 4 - protein, 480 aa, synthetic construct
MGSSHHHHHHSSGLVPRGSQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHTGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 5 - protein, 480 aa, synthetic construct
MGSSHHHHHHSSGLVPRGSQFPQLDPATLAAFSAAFRGELIWPSDADYDEARRIWNGTIDRRPALIARCTSTPDVVAAVSFARKSGLLVAVRGGGHSMAGHSVCDGGIVIDLSLMNSIKVSRRLRRARAQGGCLLGAFDTATQAHMLATPAGVVSHVGLGGLVLGGGFGWLSRKYGLSIDNLTSVEIVTADGGVLTASDTENPDLFWAVRGGGGNFGVVTAFEFDLHRVGPVRFASTYYSLDEGPQVIRAWRDHMATAPDELTWALYLRLAPPLPELPADMHGKPVICAMSCWIGDPHEGERQLESILHAGKPHGLTKATLPYRALQAYSFPGAVVPDRIYTKSGYLNELSDEATDTVLEHAADIASPFTQLELLYLGGAVARVPDDATAYPNRQSPFVTNLAAAWMDPTEDARHTAWAREGYRALAGHLSGGYVNFMNPGEADRTREAYGAAKFERLQGVKAKYDPTNLFRLNQNIPPS

SEQ ID NO: 6 - protein, 909 aa, synthetic construct
MGSSHHHHHHGSSGASEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSITSLYGSSGSSGSNLYFQSNGMTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 7 - protein, 542 aa, synthetic construct
MQACNNHQLTDAILQEFSQTLSGDLVLPTDGLYQWARLIHHTNFDGIYPVGIVFCETPEDVSKAILFARQFGLHVTARGGGHSYEGYSVTGGLLIDVSRMNSVSVNPAAMTAVVGAGAVLIDVYHGLYYPHKLSIPGGSCPSVGIAGYLLGGGVGLQSRTYGVGCDRVLEIGVVLASGEYVVASPTNHSDLYWAYRGGGGGNFGVVTHFKMQCHPVDRLSYAIITWDWEAAAPAFNAWQNWLKGLGEDYRNFSIFKFLVNAGGDGGLGPKVNLIVQFDSQSNTGTGELETLMAPLLNTAHEHIVSQTIMNGDFFEVTMEIMAGCAWPKTDLDTEFLRCHTVGNPAFPMASLPRDTYKAKSTFFADVISEKGIETCIKAIEDRFNSNLPDNSSQFTCALQFDSEGGIMGDVPKDATAFMHRDCLMHCQYLAYWPAEGDAWFDDNPDYSYCDVSNGSMQWISDAFNVLWPFGNGHAYQNYIDKEQPNWLYAYYGENVERLRTVKAKYDPDNIWKFEQSIPPAQGGSGGSGSGSGGSGSHHHHHH

SEQ ID NO: 8 - protein, 518 aa, unidentified
MGSSHHHHHHSSGLVPRGSKHIDSVAPGDIRYEDLRRGENLRFVGDPEEIHLVGSAAEIEQVLSRAVRSGKRVAVRSGGHCYEDFVANSDVRVVMDMSRLSAVGFDEERGAFAVEAGATLGAVYKTLFRVWGVTLPGGACPDVGAGGHILGGGYGPLSRMHGSIVDYLHAVEVVVVDASGDARTVIATREPSDPNHDLWWAHTGGGGGNFGVVVRYWLRTAEADVPPEPGRLLPRPPAEVLLNTTVWPWEGLDEAAFARLVRNHGRWFEQNSGPDSPWCDLYSVLALTRSQSGALAMTTQLDATGPDAEKRLETYLAAVSEGVGVQPHSDTRRLPWLHSTRWPGIAGDGDMTGRAKIKAAYARRSFDDRQIGTLYTRLTSTDYDNPAGVVALIAYGGKVNAVPADRTAVAQRDSILKIVYVTTWEDPAQDPVHVRWIRELYRDVYADTGGVPVPGGAADGAYVNYPDVDLADEEWNTSGVPWSELYYKDAYPRLQAVKARWDPRNVFRHALSVRVPPA

SEQ ID NO: 9 - DNA, 1443 bp, synthetic construct
  source 1..1443: mol_type = other DNA
atgggcagcagccatcatcatcatcatcacagcagcggcctggtgccgcgcggcagccagttcccgcagttggaccctgctactttagccgcgttctccgcagctttccgcggcgaactgatttggccatccgatgcagactacgatgaagcgcgccgtatctggaacggtactatcgaccgccgtccggctctcatcgcgcgctgtactagcaccccggacgtggttgctgcggtttccttcgcgcgcaaatctggcctgcttgtggcggttcgtggcggtggccactctatggctggtcactcggtatgcgacggtggcattgtgattgacctgtctctgatgaactccatcaaagtatcccgccgtctgcgccgtgctcgtgcgcagggtggctgcctgctcggtgctttcgacactgctacccaggcgcatatgctcgcgaccccggctggcgtcgtttcccacaccggcctgggcggtctggttctgggtggcggtttcggctggctgtcacgtaaatatggcctgagcatcgacaacctgacctctgtagaaatcgtgaccgctgacggtggcgtgctgaccgcatccgatactgagaacccggacttattctgggctgttcgcggaggaggaggtaattttggcgtcgtaaccgctttcgaattcgacttacaccgcgtcggcccagttcgtttcgcatcgacctactatagcctcgatgaaggcccgcaggtcatccgtgcttggcgtgaccacatggcgacggcaccggatgaactgacctgggcgctctatctgcgtctggcgccgccactgccggaactgcctgcagacatgcacggcaaaccggttatctgcgcaatgtcttgctggattggtgacccacatgaaggtgaacgtcagttagaatctattctgcatgccggtaaaccgcacggcctgactaaagcgacccttccgtaccgcgcactgcaggcctattccttcccgggcgcagtcgttccggaccgtatctacactaaatccgggtacctgaatgagctgtccgacgaggcgaccgataccgtgcttgaacacgcagcggatatcgcgtcgccgttcactcaacttgaactgctctacctgggcggtgccgttgctcgtgttccagatgacgcgaccgcttatcctaaccgccagtctccgtttgtgaccaacctggcggcagcttggatggatccgaccgaggatgctcgtcacaccgcttgggcgcgcgaaggttaccgtgcgctggcgggccatctgtccggtggctacgttaactttatgaacccgggtgaggcggaccgtacccgtgaagcctacggcgcggcaaaatttgagcgcctgcagggcgtgaaagctaaatatgacccgactaacctgttccgtctgaatcagaacattccgccatcctag

SEQ ID NO: 10 - DNA, 1443 bp, synthetic construct
  source 1..1443: mol_type = other DNA
atgggcagcagccatcatcatcatcatcacagcagcggcctggtgccgcgcggcagccagttcccgcagttggaccctgctactttagccgcgttctccgcagctttccgcggcgaactgatttggccatccgatgcagactacgatgaagcgcgccgtatctggaacggtactatcgaccgccgtccggctctcatcgcgcgctgtactagcaccccggacgtggttgctgcggtttccttcgcgcgcaaatctggcctgcttgtggcggttcgtggcggtggccactctatggctggtcactcggtatgcgacggtggcattgtgattgacctgtctctgatgaactccatcaaagtatcccgccgtctgcgccgtgctcgtgcgcagggtggctgcctgctcggtgctttcgacactgctacccaggcgcatatgctcgcgaccccggctggcgtcgtttcccacgtgggcctgggcggtctggttctgggtggcggtttcggctggctgtcacgtaaatatggcctgagcatcgacaacctgacctctgtagaaatcgtgaccgctgacggtggcgtgctgaccgcatccgatactgagaacccggacttattctgggctgttcgcggaggaggaggtaattttggcgtcgtaaccgctttcgaattcgacttacaccgcgtcggcccagttcgtttcgcatcgacctactatagcctcgatgaaggcccgcaggtcatccgtgcttggcgtgaccacatggcgacggcaccggatgaactgacctgggcgctctatctgcgtctggcgccgccactgccggaactgcctgcagacatgcacggcaaaccggttatctgcgcaatgtcttgctggattggtgacccacatgaaggtgaacgtcagttagaatctattctgcatgccggtaaaccgcacggcctgactaaagcgacccttccgtaccgcgcactgcaggcctattccttcccgggcgcagtcgttccggaccgtatctacactaaatccgggtacctgaatgagctgtccgacgaggcgaccgataccgtgcttgaacacgcagcggatatcgcgtcgccgttcactcaacttgaactgctctacctgggcggtgccgttgctcgtgttccagatgacgcgaccgcttatcctaaccgccagtctccgtttgtgaccaacctggcggcagcttggatggatccgaccgaggatgctcgtcacaccgcttgggcgcgcgaaggttaccgtgcgctggcgggccatctgtccggtggctacgttaactttatgaacccgggtgaggcggaccgtacccgtgaagcctacggcgcggcaaaatttgagcgcctgcagggcgtgaaagctaaatatgacccgactaacctgttccgtctgaatcagaacattccgccatcctag

SEQ ID NO: 11 - DNA, 1521 bp, synthetic construct
  source 1..1521: mol_type = other DNA
atgaccgcagatccgagcagcgaacgtagcgatatgaatgaagcagatgaagtgaacgaagttgatgaactgagcgaaaccggtcagaccagcggcaccaaaggtaaacgtccgtttacaggtcgtgttattggtccggcagatggtgaatttgatgaagcacgtcgtgtttggaatgaatgttttgcagcacgtccgaaagaaattgtttattgtgcagatacccgtgatgttgttcgtgcactgcgtgaagttcgtcagcgtggtggtccgtttcgtgttcgtagcggtggtcatagcatgagcggtctgagcgttctggatgatggtacagtgctggatgttagtggcctggatgatattcaggttagcgaagatgcaagcaccgttaccgttggtagcggtgcacatctgggtgatatttttcgtgccctgtgggcacgtggtgttaccgttccggcaggtttttgtccggaaattggtattgcaggtcatgttttaggtggtggtgcaggtattctggtgcgtagccgtggttttctgagcgatcatctggttgcactggaaatggttgatagcgaaggtcgtattgttgttgcagatcatgatagtcatcatgaactgctgtgggcaagccgtggtggtggcggtggtaattttggcattgcaaccagctttaccctgcgtacccagccgattggtgatgttaccctgtttaccattgcatgggattgggatcgtggtgccgaagcaattaaagcatggcaagaatggctggcaaccgcagatggtcgcattaatacactgtttattgcatatccgcaggaccaggatatgtttgcagccctgggttgttttgaaggtgatgcagcagaactggaaccgctgattgcaccgctggttcatgcagttgaaccgaccgaaaaagttgcagaaaccatgccgtggattgaagcactgagctttgttgaaacaatgcagggtgaagccatgaaagcaaccagcgttcgtgcaaaaggtaatctgagttttgttaccgaaccgctgggtgatcgtgccgttgaagaaatcaaaaaagcactggcacaggcaccgagccatcgtgccgaagttgttctgtatggtttaggtggcgcagttgcagcaaaaggtcgtcgtgaaaccgcatttgttcatcgtgatgcaccggttgcgctgaattatcataccgattgggatgatgaagccgaagatgatctgaattttgcctggattcagaatctgcgtgcaagcgttgcagcacataccgaaggtcgcggtagctatgttaataccattgatctgaccgttgaacattggctgtgggattattatgaagaaaatctgcctcgtctgatggccgtgaaaaaacgttatgatccggaagatgtttttcgtcatccgcagagcattccggttagcctgaccgaagcagaagcagccgaactgggtattccgcctcatattgccgaagaactgcgtgccgcacgtcagctgcgttag

SEQ ID NO: 12 - DNA, 2730 bp, synthetic construct
  source 1..2730: mol_type = other DNA
atgggtagcagccatcaccatcatcatcatggtagcagcggtgcaagcgaagaaggcaaactggttatttggattaatggcgataaaggctataatggtctggcagaagttggcaaaaaattcgaaaaagataccggcattaaagtgaccgttgaacatccggataaactggaagaaaaatttccgcaggttgcagcaaccggtgatggtccggatattatcttttgggcacatgatcgttttggtggttatgcacagagcggtctgctggcagaaattacaccggataaagcatttcaggacaaactgtatccgtttacctgggatgcagttcgctataacggtaaactgattgcatatccgattgcagttgaagcactgagcctgatctataacaaagatctgctgccgaatccgcctaaaacctgggaagaaattccggcactggataaagaactgaaagcaaaaggtaaaagcgcactgatgtttaatctgcaagaaccgtattttacctggcctctgattgcagcagatggtggctatgcattcaaatatgaaaacggcaaatacgatatcaaggatgttggtgttgataatgcgggtgcaaaagccggtctgacctttctggttgatctgatcaaaaacaaacacatgaatgccgataccgattatagcattgcagaagcagcatttaacaaaggtgaaaccgcaatgacaattaatggtccgtgggcatggtcaaatattgataccagcaaagtgaattatggtgttaccgttctgccgacatttaaaggtcagccgagcaaaccgtttgttggtgtgctgagcgcaggtattaatgcagcaagcccgaacaaagaactggcaaaagaatttctggaaaactatctgctgaccgatgaaggtctggaagcagtgaataaagataaaccgctgggtgcagttgcactgaaaagctatgaagaggaattagcaaaagatccgcgtattgcagccaccatggaaaatgcacagaaaggcgaaattatgccgaatattccgcagatgagcgcattttggtatgccgttcgtaccgcagtgattaatgccgcatcaggtcgtcagaccgttgatgaagccctgaaagatgcccagaccaatagcattaccagcctgtatggtagcagcggtagctcaggtagcaatctgtattttcagagcaatggcatgaccgcagatccgagcagcgaacgtagcgatatgaatgaagcagatgaagtgaacgaagttgatgaactgagcgaaaccggtcagaccagcggcaccaaaggtaaacgtccgtttacaggtcgtgttattggtccggcagatggtgaatttgatgaagcacgtcgtgtttggaatgaatgttttgcagcacgtccgaaagaaattgtttattgtgcagatacccgtgatgttgttcgtgcactgcgtgaagttcgtcagcgtggtggtccgtttcgtgttcgtagcggtggtcatagcatgagcggtctgagcgttctggatgatggtacagtgctggatgttagtggcctggatgatattcaggttagcgaagatgcaagcaccgttaccgttggtagcggtgcacatctgggtgatatttttcgtgccctgtgggcacgtggtgttaccgttccggcaggtttttgtccggaaattggtattgcaggtcatgttttaggtggtggtgcaggtattctggtgcgtagccgtggttttctgagcgatcatctggttgcactggaaatggttgatagcgaaggtcgtattgttgttgcagatcatgatagtcatcatgaactgctgtgggcaagccgtggtggtggcggtggtaattttggcattgcaaccagctttaccctgcgtacccagccgattggtgatgttaccctgtttaccattgcatgggattgggatcgtggtgccgaagcaattaaagcatggcaagaatggctggcaaccgcagatggtcgcattaatacactgtttattgcatatccgcaggaccaggatatgtttgcagccctgggttgttttgaaggtgatgcagcagaactggaaccgctgattgcaccgctggttcatgcagttgaaccgaccgaaaaagttgcagaaaccatgccgtggattgaagcactgagctttgttgaaacaatgcagggtgaagccatgaaagcaaccagcgttcgtgcaaaaggtaatctgagttttgttaccgaaccgctgggtgatcgtgccgttgaagaaatcaaaaaagcactggcacaggcaccgagccatcgtgccgaagttgttctgtatggtttaggtggcgcagttgcagcaaaaggtcgtcgtgaaaccgcatttgttcatcgtgatgcaccggttgcgctgaattatcataccgattgggatgatgaagccgaagatgatctgaattttgcctggattcagaatctgcgtgcaagcgttgcagcacataccgaaggtcgcggtagctatgttaataccattgatctgaccgttgaacattggctgtgggattattatgaagaaaatctgcctcgtctgatggccgtgaaaaaacgttatgatccggaagatgtttttcgtcatccgcagagcattccggttagcctgaccgaagcagaagcagccgaactgggtattccgcctcatattgccgaagaactgcgtgccgcacgtcagctgcgttag

SEQ ID NO: 13 - DNA, 1626 bp, synthetic construct
  source 1..1626: mol_type = other DNA
atgcaggcatgtaataatcatcagctgaccgatgcaatcctgcaagaattttcacagaccctgagcggtgatctggttctgccgaccgatggtctgtatcagtgggcacgtctgattcatcataccaattttgatggtatttatccggtgggcattgtgttttgtgaaacaccggaagatgttagcaaagcaattctgtttgcacgtcagtttggtctgcatgttaccgcacgtggtggtggtcatagttatgaaggttatagcgttaccggtggtctgctgattgatgttagccgtatgaatagcgttagcgttaatccggcagcaatgaccgcagttgttggtgccggtgcagttctgatcgatgtttatcatggcctgtattatccgcacaaactgagcattccaggtggtagctgtccgagcgttggtattgcaggttatctgcttggtggtggcgtgggtctgcagagccgtacctatggtgttggttgtgatcgtgttctggaaattggtgttgttctggcaagcggtgaatatgttgttgcaagcccgaccaatcatagcgatctgtattgggcatatcgtggtggcggtggtggtaattttggtgtggttacccactttaaaatgcagtgtcatccggttgatcgtctgagctatgcaattattacctgggattgggaagcagcagcaccggcatttaatgcatggcagaattggctgaaaggtctgggtgaagattatcgcaatttctcgatctttaagttcctggttaatgcaggcggtgatggtggtctgggtccgaaagttaatctgattgttcagtttgatagccagagcaataccggcaccggtgaactggaaaccctgatggcaccgctgctgaataccgcacatgaacatattgttagccagaccattatgaacggcgatttttttgaagtgaccatggaaattatggcaggttgtgcatggccgaaaaccgatctggataccgaatttctgcgttgtcataccgttggcaatccggcatttccgatggcaagcctgcctcgtgatacctataaagcaaaaagcaccttttttgccgatgtgattagcgaaaaaggtatcgaaacctgcatcaaagccattgaagatcgctttaatagcaatctgccggataatagcagccagtttacctgtgcactgcagttcgatagcgaaggtggtattatgggtgatgttccgaaagatgcaaccgcatttatgcatcgtgattgtctgatgcattgtcagtatctggcatattggcctgccgaaggtgatgcatggtttgatgataacccggattatagctattgtgatgtgagcaatggtagcatgcagtggattagtgatgcctttaatgttctgtggccgtttggtaatggtcatgcatatcagaactatatcgataaagaacagccgaactggctgtatgcatattatggtgaaaatgttgaacgtctgcgtaccgtgaaagcaaaatatgatccggataacatctggaagtttgaacagagcattccgcctgcatagggcggtagcggaggatctggtagtggctctggaggatctggtagtcaccatcatcatcaccat

SEQ ID NO: 14 - DNA, 1557 bp, synthetic construct
  source 1..1557: mol_type = other DNA
atgggcagcagccatcatcatcatcatcacagcagcggcctggtgccgcgcggcagcaaacacattgactcggttgcgccgggagacatccgttacgaggatttgcgccgcggtgaaaacttacgtttcgttggtgatccagaggaaattcacttggtcggttcggcagcggaaattgaacaggttcttagtcgcgcggtgcgcagtgggaaacgtgtcgcggtacgctctggcgggcattgctacgaagattttgttgcgaacagcgacgtgcgcgtggttatggatatgtcccgcttaagcgcagtgggctttgacgaggagcgcggcgcttttgccgtagaggctggggccacgctgggcgcagtatacaagaccttgtttcgcgtatggggagtgactctgccaggtggcgcctgtcctgatgtaggcgctggcggacatatccttggcggtggatatggtcctttgtcacgcatgcatgggtcgatcgtcgattatcttcacgctgttgaggtggtcgtcgtcgacgcttctggagacgcccgtactgtaatcgctacccgcgagccgagcgaccctaaccacgatttatggtgggcgcacactggaggtggtggtgggaacttcggggtagtcgtacgctactggcttcgtacagcggaggccgacgtacctccggagcctgggcgcctgttgccccgtccaccagctgaagtcttgctgaatactacagtgtggccctgggagggattagacgaggccgcgtttgctcgtctggtgcgtaatcacggccgttggttcgaacaaaactcgggacctgattcgccttggtgtgacctttatagtgtcttagcgttgacccgctcacagtccggtgcgttagctatgacaactcagcttgacgcaacgggacctgacgccgaaaaacgtcttgagacatatcttgcggctgttagcgaaggcgttggggttcagccccattccgatacacgccgcttgccctggttgcattcgacacgctggccaggcatcgccggcgatggcgacatgacgggacgtgcgaagattaaggctgcctatgcccgtcgcagttttgacgatcgccaaattggcacattatacacacgtctgacgagcaccgattatgataaccctgctggagtcgtggctctgattgcttatggcggaaaagtcaacgcagtacctgccgaccgtactgccgtagcgcagcgcgattccattctgaagattgtatatgttacgacttgggaagaccccgctcaagatcctgtgcatgtgcgttggatccgcgagttataccgtgacgtttacgccgacacggggggtgtgcctgttcctgggggtgcggccgatggagcttacgttaactaccccgacgtggatttggcggacgaggaatggaatacttcgggggtcccgtggagcgagttatattacaaggatgcctatcctcgtctgcaagctgtgaaagcacgctgggacccgcgcaacgttttccgtcacgctttgtctgtccgtgtcccgccagcttag

SEQ ID NO: 15 - protein, 525 aa, synthetic construct
MKHHHHHHHHGGLVPRGSHGTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 16 - protein, 492 aa, synthetic construct
ADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 17 - protein, 477 aa, synthetic construct
TSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 18 - protein, 477 aa, unidentified
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGTCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFEYRTDWDSPEDDRLNLDWVTRLRHAMAEHTTGAAYVNTIDLALENWLWAYYEENLPRLMAVKRRYDPENVFHHPHSIPGSLTAEAARAHGVPEATLKRLHDDGLLDGPLD

SEQ ID NO: 19 - protein, 473 aa, Phytohabitans suffuscus
MSTAPVFGGRIVTRLDPSYEEARQVWNAVPDHRPLEIRYCRDAQDVAAALRDALARGLPFAARSGGHSAAALSVVPDGVVIDVSPLNSISLDQDASQVTFGSGATLGAIYRTLWESGVTVPAGTCPNIGVAGHVLGGGLGILSRSRGPLVDHLIGVTMVDARGELVTADEENHSDLLWACRGGGGGNYGIATSFTLRTQPIADVTLFNITWRFDDLGPAVKAWQSWLADADHRINPFMMLYPREQDMVATVGVMEGTPDAFEPLVKPLLDAVEPSEVVIEPMSFIQAVDTVEALQGEAAVAKSVRALGSSAIIDRPLDDRALETLRRHLIDPPSHRSEVAIYGMGGKIAAKGREETAFVHRTELMAFEYRTDWDEPADDEKNVAWVRSVRQEMAEHTTGGTYTNTVDLGVENWLWGYYEENLPRLMGVKHRYDPDGVFQHAHSVPLSLTEAEASRFGIPEQIRRTLRAAGRLT

SEQ ID NO: 20 - protein, 475 aa, Streptomyces varsoviensis
MATSARFAGTVLAPGDPGFEDASRTWNACFASRPREVMVCHSTAEVADAVREVRERGVSFRVRSGGHSMAGLSSVEDGVVIDLGGLDAISLSADRSLVTVGGGTRLGDIYRELWRSGVTVPAGTCPRIGIGGHVLGGGMGVLSRSRGALVDHLAALEFVDAEGRAYRVDENEHPDLFWACRGGGGGSYGIATSYTLRTSPVDEVTLFTLSWNWERFAPAVRAWQSWLASTDDRVNTFLSLFPRQQDLVAAFGVYDGPAAEFEELLRPLVAAVRPQDQVVEPMPFIQAVDTVELLQGEAAAADQVRAQGSSAIIGGPLDGAAIATLQQFLTDPPSHRSEVAVYGMGGAIARPARTDTAFVHRTGLMAFEYRTDWDRSEDDAKNLAWIKALRHAMAPHTTGAAYVNTIDLALENWLWAYYEENLPRLIEVKRRYDPGDVFHHPHSIPLTVTEADIARYGIPEAVAARLRAAGLTDRQ

SEQ ID NO: 21 - protein, 473 aa, Streptomyces olivochromogenes
MGTPARFTGTVLTPGDAGFEDASRTWNACYASRPREVMVCHSAEAVAEALGSARGRGIPFRVRSGGHSMAGLSSVADGAIIDLGGLDAITLSEDSSLVTVGGGARLGDIYRELWKAGVTVPAGTCPRIGIGGHVLGGGMGVLSRSRGALVDHLVGLELVGADGRLRQVDDDEHADLFWACRGGGGGSFGIATSYTFRTSPISEVTIFTIAWSWAQFAPAVKAWQGWLASAEDRINTFLSLFPRQQDLLVAFGVFDGPAAEFEELLRPLVAAAEPQTLVVEPVPFIQAVDTVEALQGEAAAADQVRAQGSSAIVAEPLNEAAIATLQEFLTDPPSHRAEVAVYGMGGAIGHRARTDTAFVHRTGLMAFEYRTDWDLPEDDGKNLAWVRALRHSMAEHTTGAAYVNTIDLALENWLWAYYEENLPRLMQVKRQYDPGDVFQHPHSIPVSLTAEDIARHGIPEQVAAEIRSAGLLS

SEQ ID NO: 22 - protein, 468 aa, Streptomyces albogriseolus
MDRYPRNAGTVVSPADPDYERARRIWNNRFDCRPRAIMYPRDEAEAAAALGYARDNEVPFRIRSGGHNSEGFCTVDDGIIIDLSKLGSIAVNNDRTRAVISPGVMLGEVYANLWSVGGTIPAGVCPDIRIGGHVLGGGIGMLVRSRGLLIDNLVGLTVVDARGERLQVDADHHPDLFWACKGGGGGNYGIATSYTFEMRPISDVTIFKLQWDWQGGMAVLDAWQHWLLSADSRVNARFNVFSSTVGTVMTVGLFEGSADELKSILQPLVEKFPPRDSFVRTMPYMECVGTFSEQVPSIRAKFVPALSAGPLDADALKTLERWHKDAPTGVKTGLYGLGGEVLGSVSPDSSAFAHRDASICVEYLGHWQGAEHDREHLGWLSGVREEMDAFMTGGAYVNSPDRDLENWLHAYYGESLPRLMDVKRRYDPDNVFAFEQSIPGSLTLPEARAAGLTDTVVEDLRARGLLSA

SEQ ID NO: 23 - protein, 468 aa, Streptomyces sp.
MGAYPDTIGTVVTPGDTRYERARRLWNERFDYRPRAVVLPGDSHETAAALRYVRERGIPFRIRSGGHNSEGFSGLDDGIVIDVSRLGALAVYGDRTRATLGPGVLLGEAYAALWALGGTIPAGVCPDIRIGGHVLGGGIGMLVRSRGLLIDHLVALTLIDARGEELTVDADHHPDLLWASRGGGGGNFGIVTSYTFAMRPIGDVTLFTLEWDARDGIAALDAWQHWLSQADPRVNSRFNLFPASADRALATGLFEGGERELTALLRPLTNACRPRATSVRALPYGDSIGTWTEQVPSVRAKFTPALAEGPLGAEALRTLGRRHEKAPPGVKTGLYGLGGAVLADVPPDHSAFAHRAAFLCVEYLGHWSDPGSDAEHLDWLSGTRAEMLPFMTGGAYVNSPDLDLENWLHAYYGDGLPRLMEVKRRYDPHGLFTFEQSIPTALTRAQARAARLPGPVVADLHARGLLTD

SEQ ID NO: 24 - protein, 468 aa, Streptomyces sp.
MEEHPQHVGTVVTQADPDYEQARHIWNERFDFRPRAVFYPRNAEETAAAVTHVRDRGLPFRIRSGGHNSEGFCSVDDGVVIDLSKLASIGVNNDRTRATTGPGVLLGEVYATLAAVGGTIPAGVCPDIRIGGHVLGGGIGMLVRSRGLLIDSLVGLTLVDARGRILEVDDSSHPELMWACRGGGGGNYGIVTSYTFEMRPVSEVTLFTLEWDWNGGIGVLDTWQHWILRADTRVNARFAFLPGSTGTVMSTGLFEGPARELESILRPLVGKHPPRDSVVRTVPYAESVGTFTEPVPSIRAKFVPALAGGPLGTEALEILERRHRDAPAGVKTGLYGLGGAVLDNAPAKRSSFAHRDAPICVEYLGHWKDSAHDSEHLDWLTATREELHGFMTGGAYVNSPDRDLADWLHAYYGENLPGLMDVKRRYDPDDVFSFEQSIPTSLSPAEARAAGLTDTVVAELRGRGLLRS

SEQ ID NO: 25 - protein, 470 aa, Mycobacterium sp.
MSQTGRTISPSDPEYDEARKGWNDVYQSHPTEIIFCCNADEVASALKRCTEQQLPFRVRSGGHSLEGYSALNDGAVIDLSGMNSLVLNDDTTEVTLGPGARLGDVYSDLFANGVTVPGGTCPRVAVGGLVLGGGYGYLSRTYGMLAQRALSFEIVAADGRILTASKDEHADLFWACRGGGGGIFGIMTAVTLKTVPISDVATCRLVWSWDHFAEVFDAYQRWLPTSDSRLNTLLFLPAQHYGKIALSVLCTTTTTEELEPLLQSLLDVVPAPRETKYAVIPYIERVGNEDGLIEPQVYTRTRFGALQPYFRQPLDAAGIEALRRMHAEATGNTITIVCGAGSQVFTKMAENDPVSFPHLDALMSFDIRIDWSDPDLDHVYFDWLSAIDQELALYRSGTYLNYNDFNIDDALYPRYLDSFARLVAIKHRYDPDNTFQFPASIPPSLTEDEAERMQLPDKVIKELREYGRLT

SEQ ID NO: 26 - protein, 467 aa, Kutzneria sp.
MSRSQRVAATVTAADPGYDQARRIWNERFDCRPLAVLYPADVAEVAGAVRAARDDGVPFRIRSGGHNVEGFSVLDDGYVIDLSRLTGVTVDDDRTRGTVAAGTLLGDVYSGLWAAGVTIPAGVCPDIRIGGHVLGGGVGMLVRSRGMLVDSLVGLTMVDADGEVLEVNDDTHPDLMWACRGGGGGNFGVATSYTFHTRPIGDVTLVTAAWNFDNAITALDRWQHWAVEVDQRVNARFSLFSPGTGGGRMAVLFEGTSAEWESLARPLFEQCPPAEHTVRETAYINSVGQFSQRVPSIRAKFVPALAAGPLDAEALKILERHHRAAPDGVKTSFYGLGGAVLGADLAKRSAFAHRDALFCVEYLGHWHGAEHDAEQLGWLAGIRDELRPFMTGGAYVNSPDRDLTDWLHAYYGASLPRLMEVKRRYDPEDVFRFEQSIPVSLSPAAARAAGLTEAVVADLGARGLLTS

SEQ ID NO: 27 - protein, 500 aa, Acidovorax soli
MAHRKSPARIAKAARVAQFHALAASAADAAGGAGETSDTAIALESLAHLEHMLVGRVVLPSSPDYDQARQESNPAFQSFPKIIVYCAVENDVLECLAVARKYKVPVAVRCGGHSTAGYSVNNGMVIDLSDIQGVVLDPPNQRVHVRPGTDFDHFNGAMNHTGWHVPTGACGSVCVGGFVQGGGYGYTSRRYGIQSDLVESLRVALADGTIVTASETENPLLYWSMRGGTGGNFGVLLQVTYTMVPLDTVWAWSIGWDMSQAANVLTMMQQRYMQSGAPDALGYMMNLGYYGGQPVAMVQGMFCGTREAGLQAIAPLMEIPSAQLLVDKTGTYPDMNIWLEDHPYSLPDNLPDGIAESKASGYISRPLTTADWQSIIDYFKTSPNQWSLAYLEPYGGAINRYPIEASAFIHRRVDADLVVDVFWRNPAERAQTEAWLAGFMQLVQPFLNGHVYQNYPDASLQDFENAYWGPAYPTLQQAKRAYDPTNFFKFQQSIRLPREE

SEQ ID NO: 28 - protein, 515 aa, Bradyrhizobium sp.
MKSLGRREREKALRVQGWAGGISLDPRFTKDDLAALTMKVQGDIVLPTDAGYQQDRQLANNAFQSFPQLIVYCEVFSDIRQCLAFAQRCGLTVAVRSGGHSTAGLSVNDDMVIDLSRLRYVIVDPKAHTAVVGAGTNMGHLNAALDIYHLHVPGGSCHDVCVAGFMQGGGFGYTSRCFGLNCDNVVAIRVMLADGRIVVADDKVNRDLFWAVRGGTGGNFGILLEITYRLHEVKSVWAFRLQWPLRAGSDDTVRAARLLSIIQSEFTRKAPKELGYMTFLGWQGDMPYLIIRGMYVGPAERGKALLQPLLDVPGAEWINDRSGSYAEMMHYLVSTTPDLPQVPDLAREDKQSGYVARPLGPDDWQEVIEQFLETPNNSSMLAMEHYGGAISERHEHDMAFVHRRVDMDVYLDVFWMNEPERVDAVTFLDRFMRFMERFCNGQSYQNYPRLAQTDYRRRYWGDVFDDLLAVKRKYDPGTFFRSAQGISADPSLSATDPVSTLTGLLDGIESEADPV

SEQ ID NO: 29 - protein, 501 aa, Massilia sp.
MVSGRRRFLQVGAAGAAGAAALALAPSAVLAKPALKLGPHNLRALARDFDGRLILPGQEGYALAAWPNNARYAGVLPQAVAMCANQKDVQRCIRWAGDSGMPFAIRSGGHNYAGFSTTPGLLIDVKAMNSVTVDLKNETAWVQGGANNNDVAAVLRNYPFAIPSGRCPTVGTSGLVLGGGWGFAATHDGLTCDSLLGTELVLASSEARSINANSHKELFWAVRGGGGGNFGVHTGFTFQLHKVGKVTTFNLSWAPGKQIELLRALQALQLAYPTMISTRSKALPSAAGPRPARDTLYVQTLGQFWGGEKELREMLAPLYAIAVPQTADIYEVDYWRGRDYLLTDDPNGLYDIRCSFVHKELSEAALDNMLNWMTQWPGGSVLQENMGILFAIGGQVKAVRSDATAYVHRDSNFIFEMEASWGPIDTPETVQRQRAWLKDYYNDMQRYLQPESYVNFPNRDLKNWAQAYYGKNLEQLSAVKRHVDPGNLFRFEQSIPLAPKR

SEQ ID NO: 30 - protein, 549 aa, unidentified
MMNPTSRRTFLRATGATAIMVGATPLAAWKQACNNHQLTDAILQEFSQTLSGDLVLPTDGLYQWARLIHHTNFDGIYPVGIVFCETPEDVSKAILFARQFGLHVTARGGGHSYEGYSVTGGLLIDVSRMNSVSVNPAAMTAVVGAGAVLIDVYHGLYYPHKLSIPGGSCPSVGIAGYLLGGGVGLQSRTYGVGCDRVLEIGVVLASGEYVVASPTNHSDLYWAYRGGGGGNFGVVTHFKMQCHPVDRLSYAIITWDWEAAAPAFNAWQNWLKGLGEDYRNFSIFKFLVNAGGDGGLGPKVNLIVQFDSQSNTGTGELETLMAPLLNTAHEHIVSQTIMNGDFFEVTMEIMAGCAWPKTDLDTEFLRCHTVGNPAFPMASLPRDTYKAKSTFFADVISEKGIETCIKAIEDRFNSNLPDNSSQFTCALQFDSEGGIMGDVPKDATAFMHRDCLMHCQYLAYWPAEGDAWFDDNPDYSYCDVSNGSMQWISDAFNVLWPFGNGHAYQNYIDKEQPNWLYAYYGENVERLRTVKAKYDPDNIWKFEQSIPPA

SEQ ID NO: 31 - protein, 481 aa, Streptomyces viridochromogenes
MVTMARNRLFHAPTVGTEGRAAHAASSAAAPPRAPGHRGAKVDWDRLRQRMRGALLRPGDPGFEDARKVFNPLNDDHVPVAVAQCESTSDVSEAVRAAASRVPLAARSGGHSYVGYSAPHGGLALDLRRMSAVEVQSDGVATVGAGAPLRDVYGGVAQANRCLPAGSCFTVGVAGVTLGGGIGVLQRRFGLTCDHLVGAEMVTADGRTLTVSAARTPDLFWALRGGGGGNFGIVTQFTFATDPAPALTVFVVGFPPGKVPEVLSAWQSWISAAPRELWANCNMTGGDVPSCRVAGCFVGPSSSCNPLLDDLISRSGVLPNRRTVQDREYFSAMRFFSGSPERQSFVASSRILSEQASSPDAVAGLLVGRPGVSIILDPLGGAVADVGVQQTAFPHRKAFATAQIYASATAASEAEVTRTVRDVVAGLASLGLGGGYVNYIDPALPDWAQAYYGPNLQRLRSVAHDYDPDGVFDFPQGLTRT

SEQ ID NO: 32 - protein, 503 aa, Kibdelosporangium sp.
MINRRTLLRTGLGTAGALLLAPATAAASTRRTRWERLSGCLSGDVVLPSDPAYTQARQLDSAAFDHVLPQAIAYCENVGDVRTCVRFAQDNDIAVAVRSGGHSAAGYSTTTGMVLDVSRLSRIDVGASSVRLGPGVQGIDLLSTLSQRGLSAITGNCPTVGMGGYLQGGGVGPLSRKYGVASDRLLSAEVVLADGRVARCSPTREPDLFWALRGGGGGNFGVVTQYEIAPVTTTRTVSFSLSWPYARAAEVIAAFLPWLASAPDDLTANLTVMNQNAAPGSQPSLFVSGSWFGGPVATVDPHLTALVSAVGSAPASRSADDLTYFDAMMRMFGCGTKTVQQCHRVGYNLEATLPRQSFVLARGRLADRPMTSSAIGEALAAYEADPAGGQFRVMSWGGLGGRINRVAPSATAYAHRTAQHYFTYTAGVRTAAPPDEVKASIESWTAKGFAVARRHGNGEGQVNFIDSTLPDWRRAYYGGNHSRLQWIKWRYDPHRFFRFAQSI

SEQ ID NO: 33 - protein, 505 aa, unidentified
MTSRRSFLVGAGATALAVTAFQGSSAARTASGGKWHQLRRHLRGRLVLPSDPDYRRAKQLYLIQFDDSRPRAIAYCASAADVALCLRFAQDHALPIAVRSGGHSAGGYSTTEGLVIDVSRIDSTVLGRDTATIGPGAQLVDITNTLAPAGLTISGGYCPTVAAGGYFAGGGMGLFTRSIGMASDKVTSAEVVLADGSVVTASPRRNSDLYWAVRGGGGGNFGVVTSYEVTPTPLTDLAACNLSWRFDQALDMLDGWARWLVDAPRAIGSGLLVTLEDAAPGKVPMATIFLGSVDTGGGFDGEIDRLVSLIGHAPSFRQTHTAPYQDILMDLYQCGGLTVQQCHRADTSPGGQLTRPAFGLWRSRLFGEDAMPREGWAKALSVLDSERQAGQMRQLQVVAIGGAVNSVSRGATAYVHRDSLFSASFLTSNSVAPVPDEAKESAGRFVDAGFAVLDPYSNGETYQNFYDSRLPDWQSSYYAENYPRLKRVKRSYDPHNIFRFAQSIR

SEQ ID NO: 34 - protein, 506 aa, synthetic construct
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPRDQDMFAALGLFEGDAAELEPLIAPLVHAVEPTEKVAETMPWIEALSFYETMQGEAMKATSVRAKGNLSFVTEPLGDRAVEEIKKALAQAPSHRAQVILYGLGGAVAAKGRRETAFVHRDAPVALWYHTAWDDEAEDDLNFAWIQNLRASVAAHTEGRGSYVNFIDLTVEHWLWDYYEENLPRLMAVKKRYDPEDVFRHPQSIPVSLTEAEAAELGIPPHIAEELRAARQLR

SEQ ID NO: 35 - protein, 506 aa, synthetic construct
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTMFHAYPRDQDMFAALGLFEGDAAELEPLIAPLVHAVEPTEKVAETMPWIEALSFYETMQGEAMKATSVRAKGNLSFVTEPLGDRAVEEIKKALAQAPSHRAQVILYGLGGAVAAKGRRETAFVHRDAPVALWYHTAWDDEAEDDLNFAWIQNLRASVAAHTEGRGSYVNFIDLTVEHWLWDYYEENLPRLMAVKKRYDPEDVFRHPQSIPVSLTEAEAAELGIPPHIAEELRAARQLR

SEQ ID NO: 36 - protein, 506 aa, synthetic construct
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTMFHCYPRDQDMFAALGLFEGDAAELEPLIAPLVHAVEPTEKVAETMPWIEALSFYETMQGEAMKATSVRNKGNLSFVTEPLGDRAVEEIKKALAQAPSHRAQVILYGLGGAVAAKGRRETAFVHRDAPVALWYHTAWDDEAEDDLNFAWIQNLRASVAAHTEGRGSYVNFIDLTVEHWLWDYYEENLPRLMAVKKRYDPEDVFRHPQSIPVSLTEAEAAELGIPPHIAEELRAARQLR

SEQ ID NO: 37 - protein, 506 aa, synthetic construct
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPAIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTMFHCYPRDQDMFAALGLFEGDAAELEPLIAPLVHAVEPTEKVAETMPWIEALSFYETMQGEAMKATSVRNKGNLSFVTEPLGDRAVEEIKKALAQAPSHRAQVILYGLGGAVAAKGRRETAFVHRDAPVALWYHTAWDDEAEDDLNFAWIQNLRASVAAHTEGRGSYVNFIDLDVEHWLWDYYEENLPRLMAVKKRYDPEDVFRHPQSIPVSLTEAEAAELGIPPHIAEELRAARQLR